(19) United States
(12) Patent Application Publication
(10) Pub. No.: US 2022/0233660 A1
(43) Pub. Date: Jul. 28, 2022

(54) IMMUNOGENETIC CANCER SCREENING TEST

(71) Applicant: TREOS BIO LIMITED, London (GB)

(72) Inventors: Julianna LISZEWICZ, Balatonalmádi (HU); Levente MOLNAR, Felsopakony (HU); Eniko TOKE, Felsopakony (HU); József TOTH, Gyor (HU); Orsolya LORINCZ, Budapest (HU); Zsolt CSISZOVSZKI, Budapest (HU); Eszter SOMOGYI, Balatonalmádi (HU); Katalin PANTYA, Budapest (HU); Péter PÁLES, Budapest (HU); István MIKLÓS, Budapest (HU); Mónika MEGYESI, Mezokeresztes (HU)

(21) Appl. No.: 17/250,722

(22) PCT Filed: Sep. 3, 2019
(86) PCT No.: PCT/EP2019/073478

§ 371 Date: Sep. 23, 2021

(30) Foreign Application Priority Data
Sep. 4, 2018 (GB) 1814361.0

(51) Int. Cl. A61K 39/00; C12Q 1/6881; G16B 20/20; G16B 20/30; G01N 33/574; A61P 35/00

(52) U.S. Cl. CPC A61K 39/0011; C12Q 1/6881; G16B 20/20; C12Q 2600/112; G01N 33/57484; A61P 35/00; C12Q 2600/156; G16B 20/30

(57) ABSTRACT

The disclosure relates to a method for determining the risk that a human subject will develop a cancer, the method comprising quantifying the HLA triplets (HEAT) of the subject that are capable of binding to T cell epitopes in the amino acid sequence of tumor associated antigens. The disclosure also relates to methods of treating subjects who are determined to have an elevated risk of developing cancer.

### CROSS-REFERENCE

This application is the U.S. National Stage entry of International Application No. PCT/EP2019/073478, filed on Sep. 3, 2019, which claims the benefit of and priority to UK Application No. 1814361.0, filed on Sep. 4, 2018, each of which are incorporated herein by reference in their entireties.

### SEQUENCE LISTING

The instant application contains a Sequence Listing which has been filed electronically in ASCII format and is hereby incorporated by reference in its entirety. Said ASCII copy, created Apr. 20, 2021, is named TBL_005_SL.txt and is 7,856 bytes in size.

### FIELD

Provided herein are methods for determining the risk that a subject will develop a cancer based on their HLA class I genotype. Further provided herein are methods of treating cancer, particularly prophylactic treatment of subjects that have determined to have an elevated risk of developing a cancer.

### BACKGROUND

Screening, where possible, and early diagnosis are critically important to prevent metastatic disease and improve prognosis for many cancers.

Heritable mutations can increase the risk of developing cancers, but known genetic factors do not fully account for the genetic contribution to cancer development risk. For example, mutations in BRCA1, BRCA2 have been identified in 5% of breast cancer cases in the general population but close to 50% of these cases developed breast cancer. Over the last decade, efforts to explain the missing heritability of developing cancer have focused on discovery of high-risk genes and identification of common genetic variants.

There remains, however, a need in the art to better identify individuals who are at elevated genetic risk of developing a cancer.

### SUMMARY

Provided herein are methods relating to a subject's human leukocyte antigen (HLA) class I genotype as a predictor for cancer development.

In antigen presenting cells (APC) protein antigens, including tumour associated antigens (TAA), are processed into peptides. These peptides bind to HLA molecules and are presented on the cell surface as peptide-HLA complexes to T cells. Different individuals express different HLA molecules, and different HLA molecules present different peptides. A TAA epitope that binds to a single HLA class I allele expressed in a subject is essential, but not sufficient to induce tumor specific T cell responses. Instead tumour specific T cell responses are optimally activated when an epitope of the TAA is recognised and presented by the HLA molecules encoded by at least three HLA class I genes (referred to herein as a HLA triplet or “HLAT”) of an individual (PCT/EP2018/055231, PCT/EP2018/055232, PCT/EP2018/055230, EP 3370065 and EP 3369431).

The inventors have developed a binary classifier that is able to separate subjects having cancer from a background population. Using this classifier, the inventors were able to demonstrate a clear association between HLA genotype and cancer risk. These findings confirm the central role of tumor specific T cell responses in the control of tumor growth and mean that HLA genotype analysis may be used to improve diagnostic tests for the early identification of subjects at a high risk of developing cancer.

Accordingly, in a first aspect the disclosure provides a method for determining the risk that a human subject will develop a cancer, the method comprising quantifying the HLA triplets (HLAT) of the subject that are capable of binding to T cell epitopes in the amino acid sequence of tumor associated antigens (TAAs), wherein each HLA of a HLAT is capable of binding to the same T cell epitope, and determining the risk that the subject will develop a cancer, wherein, with respect to a TAA, a lower number of HLATs capable of binding to T cell epitopes of the TAA corresponds to a higher risk that the subject will develop cancer.

The findings described herein also suggest that the risk of cancer can be reduced by using vaccines that are personalised to effectively activate a subject's immune system to kill tumor cells.

Accordingly, in a further aspect the disclosure provides a method of treating cancer in a subject, wherein the subject has been determined to have an elevated risk of developing cancer using the method above, and wherein the method of treatment comprises administering to the subject one or more peptides or one of more polynucleic acids or vectors that encode one or more peptides, that comprise an amino acid sequence that (i) is a fragment of a TAA; and (ii) comprises a T cell epitope capable of binding to HLAT of the subject.

In further aspects, the disclosure provides
a peptide, or polynucleic acids or vectors that encode a peptide, for use in a method of treating cancer in a specific human subject, wherein the peptides comprises an amino acid sequence that (i) is a fragment of a TAA; and (ii) comprises a T cell epitope capable of binding to an HLAT of the subject; and
a peptide, or polynucleic acids or vectors that encode a peptide for use in the manufacture of a medicament for treating cancer in a specific human subject, wherein the peptides comprises an amino acid sequence that (i) is a fragment of a TAA; and (ii) comprises a T cell epitope capable of binding to an HLAT of the subject.

In a further aspect the disclosure provides a system for determining the risk that a human subject will develop a cancer, the system comprising:
(i) a storage module configured to store data comprising the HLA class I genotype of a subject and the amino acid sequences of TAAs;
(ii) a computation module configured to quantify the HLAT of the subject that are capable of binding to T cell epitopes in the amino acid sequence of the TAAs, wherein each HLA of a HLAT is capable of binding to the same T cell epitope; and
(iii) an output module configured to display an indication of the risk that the subject will develop a cancer and/or a recommended treatment for the subject.
(iv)

The methods and compositions of the present disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “a peptide” includes two or more such peptides.

Section headings are used herein for convenience only and are not to be construed as limiting in any way.

### DESCRIPTION OF THE FIGURES

FIG. 1

ROC curve of HLA restricted PEPI biomarkers.

FIG. 2

ROC curve of ≥1 PEPI3+ Test for the determination of the diagnostic accuracy. AUC=0.73 classifies a fair diagnostic value for the PEPI biomarker.

FIG. 3

The average total HLAT Score of 48 TSAs in the different ethnic populations. Ethnic groups in far East-Asia and in the Pacific region clearly have higher HLAT numbers than the rest of the word. Ethnic groups that can be associated to countries are highlighted with black. The encoding on they axis: 1: Irish, 2: North America (Eu), 3: Czech, 4: Finn, 5: Georgian, 6: Mexican, 7: Ugandan, 8: North America (Hi), 9: New Delhi, 10: Kurdish, 11: Bulgarian, 12: Brazilian (Af, Eu), 13: Arab Druze, 14: North America (Af), 15: Tamil, 16: Amerindian, 17: Zambian, 18: Kenyan, 19: Tuva, 20: Guarani-Nandewa, 21: Kenyan Lowlander, 22: Shona, 23: Guarani-Kaiowa, 24: Zulu, 25: Doggon, 26: Saisiat, 27: Israeli Jews, 28: Canoncito, 29: North America (As), 30: Korean, 31: Groote Eylandt, 32: Toroko, 33: Siraya, 34: Cape York, 35: Okinawan, 36: Bari, 37: Kenyan Highlander, 38: Hakka, 39: Atayal, 40: Chinese, 41: Filipino, 42: Minnan, 43: Yupik, 44: Kimberley, 45: Javanese Indonesian, 46: Ivatan, 47: Thai, 48: Malay, 49: Tsou, 50: Ami, 51: Bunun, 52: Yuendumu, 53: Pazeh, 54: Thao, 55: American Samoa, 56: Rukai, 57: Paiwan, 58: Puyuma, 59: Yami

FIG. 4

The incidence rate in countries with low HLAT Score (s<75) and with high HLAT Score (s>75). The averages are indicated with a horizontal black bar. Standard errors are indicated with vertical bars. The difference between the incidence rates are very significant (p<0.0001).

FIG. 5

ROC curve of the immunological predictor (HLAT Score) classifying melanoma patients compared to the general populations. AUC=0.645; the solid black line is the ROC curve, the x=y line is indicated with dotted grey for sake of comparison.

FIG. 6

The relative immunological risk of developing melanoma in five, equally large subpopulations. The HLAT Score ranges defining the subpopulations are presented on the horizontal axis. The black bars indicate the 95% confidence intervals. The difference between the first and last subgroup is significant (p=0.001).

FIG. 7

The relative immunological risk of developing a cancer in five, equally large subpopulations. The HLAT Score ranges defining the subpopulations are presented on the horizontal axis. The black bars indicate the 95% confidence intervals. A. non-small cell lung cancer; B. renal cell carcinoma; C. colorectal cancer.

FIG. 8

The relative risk (RR) of developing melanoma in five equal-size subgroups. The HLA-score (s) ranges defining the subgroups are shown on the x-axis. The black bars indicate the 95% confidence intervals. The difference between the first and last subgroups is significant (p<0.05).

FIG. 9

Positive correlation between the number of antigens (n=7) resulting in vaccine-specific T cell responses (in 10 patients) and HLAT Score calculated for the panel of 48 TSAs.

FIG. 10

The mean HLA-score in 59 different countries and ethnic populations. Ethnic groups that can be associated with countries as the country's dominant ethnicity are highlighted in black. The ethnicities encoded on the y axis: 1, Irish; 2, North America (Eu); 3, Czech; 4, Finnish; 5, Brazilian (Af, Eu); 6, Georgian; 7, Arab Druze; 8, Guarani-Kaiowa; 9, Ugandan; 10, North America (Hi); 11, New Delhi; 12, Bulgarian; 13, North America (Af); 14, Guarani-Nandewa; 15, Kurdish; 16, Israeli Jews; 17, Mexican; 18, Tamil; 19, Kenyan; 20, Kenyan Lowlander; 21, Zambian; 22, Doggon; 23, Amerindian; 24, Shona; 25, Kenyan Highlander; 26, Zulu; 27, Canoncito; 28, Tuva; 29, Saisiat; 30, Javanese Indonesian; 31, Filipino; 32, North America (As); 33, Cape York; 34, Malay; 35, Korean; 36, Thai; 37, Hakka; 38, Okinawan; 39, Chinese; 40, Groote Eylandt; 41, Minnan; 42, Ivatan; 43, Bari; 44, Kimberley (Australia); 45, Toroko; 46, Yuendumu; 47, Atayal; 48, Siraya; 49, American Samoa; 50, Yupik; 51, Pazeh; 52, Bunun; 53, Yami; 54, Tsou; 55, Ami; 56, Thao; 57, Rukai; 58, Paiwan; 59, Puyuma. Here Eu denotes European, non-Hispanic, Hs denotes Hispanic, Af means African and As means Asian.

FIG. 11

Correlation between the melanoma incidence rate and mean HLA-scores in ethnic populations. The correlation is significant (p<0.001, transformed t score is 4.25, df=18). ASRW: age-standardized rate by world standard population.

FIG. 12

Single HLA allele or non-complete HLA genotype has a limitation in genotype-based separation of UNPC population from non-UNPC population. A*02:01/B*18:01 AUC=0.556 (not significant).

FIG. 13

OBERTO trial design (NCT03391232)

FIG. 14

Antigen expression in CRC cohort of OBERTO trial (n=10). A: Expression frequencies of PolyPEPI1018 source antigens determined based on 2391 biopsies. B: PolyPEPI1018 vaccine design specified as 3 out of 7 TSAs are expressed in CRC tumors with above 95% probability. C: In average, 4 out of the 10 patients had pre-existing immune responses against each target antigens, referring to the real expression of the TSAs in the tumors of the patients. D: 7 out of the 10 patients had pre-existing immune responses against minimum of 1 TSA, in average against 3 different TSAs.

FIG. 15

Immunogenicity of PolyPEPI1018 in CRC patients confirms proper target antigen and target peptide selection. Upper part: target peptide selection and peptide design of PolyPEPI1018 vaccine composition. Two 15mers from CRC specific CTA (TSA) selected to contain 9mer PEPI3+ predominant in representative Model population. Table: PolyPEPI1018 vaccine has been retrospectively tested during a preclinical study in a CRC cohort and was proven to be immunogenic in all tested individuals for at least one antigen by generating PEPI3+s. Clinical immune responses were measured specific for at least one antigen in 90% of patients, and multi-antigen immune responses were also found in 90% of patients against at least 2, and in 80% of patients against at least 3 antigens as tested with IFNy fluorospot assay specifically measured for the vaccine-comprising peptides.

FIG. 16

Clinical response for PolyPEPI1018 treatment. A: Swimmer plot of clinical responses of OBERTO trial (NCT03391232). B: Association progression free survival (PFS) and AGP count. C: Association tumour volume and AGP count.

FIG. 17

Probability of vaccine antigen expression in the Patient-A's tumor cells. There is over 95% probability that 5 out of the 13 target antigens in the vaccine regimen is expressed in the patient's tumor. Consequently, the 13 peptide vaccines together can induce immune responses against at least 5 ovarian cancer antigens with 95% probability (AGP95). It has 84% probability that each peptide will induce immune responses in the Patient-A. AGP50 is the mean (expected value)=7.9 (it is a measure of the effectiveness of the vaccine in attacking the tumor of Patient-A).

FIG. 18

Treatment schedule of Patient-A.

FIG. 19

T cell responses of patient-A. A. Left: Vaccine peptide-specific T cell responses (20-mers). right: CD8+ cytotoxic T cell responses (9-mers). Predicted T cell responses are confirmed by bioassay.

FIG. 20

MRI findings of Patient-A treated with personalised (PIT) vaccine. This late stage, heavily pretreated ovarian cancer patient had an unexpected objective response after the PIT vaccine treatment. These MRI findings suggest that PIT vaccine in combination with chemotherapy significantly reduced her tumor burden.

FIG. 21

Probability of vaccine antigen expression in the Patient-B's tumor cells and treatment schedule of Patent-B. A: There is over 95% probability that 4 out of the 13 target antigens in the vaccine is expressed in the patient's tumor. B: Consequently, the 12 peptide vaccines together can induce immune responses against at least 4 breast cancer antigens with 95% probability (AGP95). It has 84% probability that each peptide will induce immune responses in the Patient-B. AGP50=6.45; it is a measure of the effectiveness of the vaccine in attacking the tumor of Patient-B. C: Treatment schedule of Patient-B.

FIG. 22

T cell responses of Patient-A. Left: Vaccine peptide-specific T cell responses (20-mers) of P. Right: Kinetic of vaccine-specific CD8+ cytotoxic T cell responses (9-mers). Predicted T cell responses are confirmed by bioassay.

FIG. 23

Treatment schedule of Patient-C.

FIG. 24

T cell responses of Patient-C. A: Vaccine peptide-specific T cell responses (20-mers). B: Vaccine peptide-specific CD8+ T cell responses (9-mers). C-D: Kinetics of vaccine-specific CD4+ T cells and CD8+ cytotoxic T cell responses (9-mers), respectively. Long lasting immune responses both CD4 and CD 8 T cell specific are present after 14 months.

FIG. 25

Treatment schedule of Patient-D.

FIG. 26

Immune responses of Patient-D for PIT treatment. A: CD4+ specific T cell responses (20mer) and B: CD8+ T cell specific T cell responses (9mer). 0.5-4 months refer to the timespan following the last vaccination until PBMC sample collection.

### DESCRIPTION OF THE SEQUENCES

SEQ ID Nos: 1-13 set forth sequences of personalized vaccine of Patient-A and are described in Table 23.
SEQ ID Nos: 14-25 set forth sequences of personalized vaccine of Patient-B and are described in Table 25.
SEQ ID No: 26 sets forth the 30 amino acid CRC P3 peptide, FIG. 15.

### DETAILED DESCRIPTION

### HLA Genotypes

HLAs are encoded by the most polymorphic genes of the human genome. Each person has a maternal and a paternal allele for the three HLA class I molecules (HLA-A*, HLA-B*, HLA-C*) and four HLA class II molecules (HLA-DP*, HLA-DQ*, HLA-DRB1*, HLA-DRB3*/4*/5*). Practically, each person expresses a different combination of 6 HLA class I and 8 HLA class II molecules that present different epitopes from the same protein antigen.

The nomenclature used to designate the amino acid sequence of the HLA molecule is as follows: gene name*allele:protein number, which, for instance, can look like: HLA-A*02:25. In this example, “02” refers to the allele. In most instances, alleles are defined by serotypes—meaning that the proteins of a given allele will not react with each other in serological assays. Protein numbers (“25” in the example above) are assigned consecutively as the protein is discovered. A new protein number is assigned for any protein with a different amino acid sequence determining the binding specificity to non-self antigenic peptides (e.g. even a one amino acid change in sequence is considered a different protein number). Further information on the nucleic acid sequence of a given locus may be appended to the HLA nomenclature, but such information is not required for the methods described herein.

The HLA class I genotype or HLA class II genotype of an individual may refer to the actual amino acid sequence of each class I or class II HLA of an individual, or may refer to the nomenclature, as described above, that designates, minimally, the allele and protein number of each HLA gene. In some embodiments, the HLA genotype of an individual is obtained or determined by assaying a biological sample from the individual. The biological sample typically contains subject DNA. The biological sample may be, for example, a blood, serum, plasma, saliva, urine, expiration, cell or tissue sample. In some embodiments the biological sample is a saliva sample. In some embodiments the biological sample is a buccal swab sample. An HLA genotype may be obtained or determined using any suitable method. For example, the sequence may be determined via sequencing the HLA gene loci using methods and protocols known in the art. In some embodiments, the HLA genotype is determined using sequence specific primer (SSP) technologies. In some embodiments, the HLA genotype is determined using sequence specific oligonucleotide (SSO) technologies. In some embodiments, the HLA genotype is determined using sequence based typing (SBT) technologies. In some embodiments, the HLA genotype is determined using next generation sequencing. Alternatively, the HLA set of an individual may be stored in a database and accessed using methods known in the art.

### HLA-Epitope Binding

A given HLA of a subject will only present to T cells a limited number of different peptides produced by the processing of protein antigens in an APC. As used herein, “display” or “present”, when used in relation to HLA, references the binding between a peptide (epitope) and an HLA. In this regard, to “display” or “present” a peptide is synonymous with “binding” a peptide.

As used herein, the term “epitope” or “T cell epitope” refers to a sequence of contiguous amino acids contained within a protein antigen that possesses a binding affinity for (is capable of binding to) one or more HLAs. An epitope is HLA- and antigen-specific (HLA-epitope pairs, predicted with known methods), but not subject specific.

The term “personal epitope”, or “PEPI” as used herein distinguishes a subject-specific epitope from an HLA specific epitope. A “PEPI” is a fragment of a polypeptide consisting of a sequence of contiguous amino acids of the polypeptide that is a T cell epitope capable of binding to one or more HLA class I molecules of a specific human subject. In other words a “PEPI” is a T cell epitope that is recognised by the HLA class I set of a specific individual. In contrast to an “epitope”, PEPIs are specific to an individual because different individuals have different HLA molecules which each bind to different T cell epitopes. In appropriate cases a “PEPI” may also refer to a fragment of a polypeptide consisting of a sequence of contiguous amino acids of the polypeptide that is a T cell epitope capable of binding to one or more HLA class II molecules of a specific human subject.

“PEPI1” as used herein refers to a peptide, or a fragment of a polypeptide, that can bind to one HLA class I molecule (or, in specific contexts, HLA class II molecule) of an individual. “PEPI1+” refers to a peptide, or a fragment of a polypeptide, that can bind to one or more HLA class I molecule of an individual.

“PEPI2” refers to a peptide, or a fragment of a polypeptide, that can bind to two HLA class I (or II) molecules of an individual. “PEPI2+” refers to a peptide, or a fragment of a polypeptide, that can bind to two or more HLA class I (or II) molecules of an individual, i.e. a fragment identified according to a method disclosed herein.

“PEPI3” refers to a peptide, or a fragment of a polypeptide, that can bind to three HLA class I (or II) molecules of an individual. “PEPI3+” refers to a peptide, or a fragment of a polypeptide, that can bind to three or more HLA class I (or II) molecules of an individual.

“PEPI4” refers to a peptide, or a fragment of a polypeptide, that can bind to four HLA class I (or II) molecules of an individual. “PEPI4+” refers to a peptide, or a fragment of a polypeptide, that can bind to four or more HLA class I (or II) molecules of an individual.

“PEPI5” refers to a peptide, or a fragment of a polypeptide, that can bind to five HLA class I (or II) molecules of an individual. “PEPI5+” refers to a peptide, or a fragment of a polypeptide, that can bind to five or more HLA class I (or II) molecules of an individual.

“PEPI6” refers to a peptide, or a fragment of a polypeptide, that can bind to all six HLA class I (or six HLA class II) molecules of an individual.

Generally speaking, epitopes presented by HLA class I molecules are about nine amino acids long. For the purposes of this disclosure, however, an epitope may be more or less than nine amino acids long, as long as the epitope is capable of binding HLA. For example, an epitope that is capable of being presented by (binding to) one or more HLA class I molecules may be between 7, or 8 or 9 and 9 or 10 or 11 amino acids long.

Using techniques known in the art, it is possible to determine the epitopes that will bind to a known HLA. Any suitable method may be used, provided that the same method is used to determine multiple HLA-epitope binding pairs that are directly compared. For example, biochemical analysis may be used. It is also possible to use lists of epitopes known to be bound by a given HLA. It is also possible to use predictive or modelling software to determine which epitopes may be bound by a given HLA. Examples are provided in Table 1. In some cases a T cell epitope is capable of binding to a given HLA if it has an IC50 or predicted IC50 of less than 5000 nM, less than 2000 nM, less than 1000 nM, or less than 500 nM.

TABLE 1
Example software for determining epitope-HLA binding

| | WEB ADDRESS |
|---|---|
| EPITOPE PREDICTION TOOLS | |
| BIMAS, NIH | bimas.cit.nih.gov/molbio/hla_bind/ |
| PPAPROC, Tubingen Univ. | |
| MHCPred, Edward Jenner Inst. | |
| of Vaccine Res. | |
| EpiJen, Edward Jenner Inst. | ddg-pharmfac.net/epijen/EpiJen/EpiJen.htm |
| of Vaccine Res. | |
| NetMHC, Center for Biological | cbs.dtu.dk/services/NetMHC/ |
| Sequence Analysis | |
| SVMHC, Tubingen Univ. | abi.inf.uni-tuebingen.de/Services/SVMHC/ |
| SYFPEITHI, Biomedical | syfpeithi.de/bin/MHCServer.dll/EpitopePrediction.htm |
| Informatics, Heidelberg | |
| ETK EPITOOLKIT, Tubingen Univ. | etk.informatik.uni-tuebingen.de/epipred/ |
| PREDEP, Hebrew Univ. Jerusalem | margalit.huji.ac.il/Teppred/mhc-bind/index.html |
| RANKPEP, MIF Bioinformatics | bio.dfci.harvard.edu/RANKPEP/ |
| IEDB, Immune Epitope Database | tools.immuneepitope.org/main/html/tcell_tools.html |
| EPITOPE DATABASES | |
| MHCBN, Institute of Microbial | imtech.res.in/raghava/mhcbn/ |
| Technology, Chandigarh, INDIA | |
| SYFPEITHI, Biomedical | syfpeithi.de/ |
| Informatics, Heidelberg | |
| AntiJen, Edward Jenner Inst. | ddg-pharmfac.net/antijen/AntiJen/antijenhomepage.htm |
| of Vaccine Res. | |
| EPIMHC database of MHC ligands, | immunax.dfci.harvard.edu/epimhc/ |
| MIF Bioinformatics | |
| IEDB, Immune Epitope Database | iedb.org/ |

HLA molecules regulate T cell responses. Until recently, the triggering of an immune response to individual epitopes was thought to be determined by recognition of the epitope by the product of single HLA allele, i.e. HLA-restricted epitopes. However, HLA-restricted epitopes induce T cell responses in only a fraction of individuals. Peptides that activate a T cell response in one individual are inactive in others despite HLA allele matching. Therefore, it was previously unknown how an individual's HLA molecules present the antigen-derived epitopes that positively activate T cell responses.

As described herein multiple HLA expressed by an individual need to present the same peptide in order to trigger a T cell response. Therefore the fragments of a polypeptide antigen (epitopes) that are immunogenic for a specific individual (PEPIs) are those that can bind to multiple class I (activate cytotoxic T cells) or class II (activate helper T cells) HLAs expressed by that individual. This discovery is described in PCT/EP2018/055231, PCT/EP2018/055232, PCT/EP2018/055230, EP 3370065 and EP 3369431

A “HLA triplet” or “HLAT” or “any combination HLAT” as referred to herein is any combination of three out of the six HLA class I alleles that are expressed by a human subject. An HLAT is capable of binding to a specific PEPI if all three HLA alleles of the triplet is capable of binding to the PEPI. The “HLAT number” is the total number of HLAT, made up of any combination of three HLA alleles of a subject, that are capable of binding to one or more defined polypeptides or polypeptide fragments, for example one or more antigen or a PEPI. For example, if three out of the six HLA class I alleles of a subject are able to bind to a specific PEPI then the HLAT number is one. If four out of the six HLA class I alleles of a subject are able to bind to a specific PEPI then the HLAT number is four (four combinations of any three out of four binding HLA alleles). If five out of the six HLA class I alleles of a subject are able to bind to a specific PEPI then the HLAT number is ten (ten combinations of any three out of five binding HLA alleles). If three out of the six HLA class I alleles of a subject are able to bind to a first PEPI in a polypeptide, and the same or a different combination of three out of the six HLA class I alleles of the subject are able to bind to a second PEPI in a polypeptide, then the HLAT number is two, and so on.

Some subjects may have two HLA alleles that encode the same HLA molecule (for example, two copies for HLA-A*02:25 in case of homozygosity). The HLA molecules encoded by these alleles bind all of the same T cell epitopes. For the purposes of this disclosure the HLA that are encoded by different alleles are different HLA, even if the two alleles are the same. “In other words, “binding to at least three HLA molecules of the subject” and the like could otherwise be expressed as “binding to the HLA molecules encoded by at least three HLA alleles of the subject”.

### Determining Cancer Risk

Provided herein are methods for determining the risk that a subject will develop a cancer based on their HLA class I genotype and its ability to recognise tumor-associated antigens. Because of the way that HLAT regulate T cell responses, the class I HLA genotype of a subject may represent an inherent genetic cancer risk determining factor: some subjects who inherited certain HLA genes from parents can mount broad T cell responses that effectively kill tumor cells; others with HLA genes that can recognize only few tumor antigens have poor defence against tumor cells. Based on the 6 inherited HLA alleles, the parents and the offspring have different HLA allele set. Since HLAT binding PEPIs induce T cell responses in a subject, tumor specific T cell responses of the parents are not directly inherited to the offspring.

According to the present disclosure, the presence in a TAA of an amino acid sequence that is a T cell epitope (PEPI) capable of binding to a HLAT of a subject indicates that expression of the TAA in the subject will elicit a T cell response. The greater number of HLAT that are capable of binding to epitopes of the TAA, the more effective the T cell response of the subject to expression of the TAA, and the more effective the subject will be at killing cancer cells that express the TAA. Conversely a lower number of HLAT that are capable of binding to epitopes of a TAA, the less effective the T cell response of the subject to expression of the TAA, and the less effective the subject will be at killing cancer cells that express the TAA. Tumours only arise in a subject when cancer cells that express TAAs are not detected and killed by the immune responses of the subject. Accordingly HLA genotype may represent either a genetic risk or a protective factor to the development of cancer in a subject. A higher number of HLATs capable of binding to T cell epitopes of a TAA may correspond to a lower risk that the subject will develop a tumor (cancer) that expresses the TAA. A lower number of HLATs capable of binding to T cell epitopes of a TAA may correspond to a higher risk that the subject will develop a tumor (cancer) that expresses the TAA.

In some cases the cancer is a particular type of cancer or cancer of a particular cell type of tissue. In some cases the cancer is a solid tumour. In some cases the cancer is a carcinoma, sarcoma, lymphoma, leukemia, germ cell tumor, or blastoma. The cancer may be a hormone related or dependent cancer (e.g., an estrogen or androgen related cancer) or a non-hormone related or dependent cancer. The tumor may be malignant or benign. The cancer may be metastatic or non-metastatic. The cancer may or may not be associated with a viral infection or viral oncogenes. In some cases the cancer is one or more selected from melanoma, lung cancer, renal cell cancer, colorectal cancer, bladder cancer, glioma, head and neck cancer, ovarian cancer, non-melanoma skin cancer, prostate cancer, kidney cancer, stomach cancer, liver cancer, cervix uteri cancer, oesophagus cancer, non-Hodgkin lymphoma, leukemia, pancreatic cancer, corpus uteri cancer, lip cancer, oral cavity cancer, thyroid cancer, brain cancer, nervous system cancer, gallbladder cancer, larynx cancer, pharynx cancer, myeloma, nasopharynx cancer, Hodgkin lymphoma, testis cancer, breast cancer, gastric cancer, bladder cancer, colorectal cancer, renal cell cancer, hepatocellular cancer, pediactric cancer and Kaposi sarcoma.

In other cases the method may be used to determine the risk that a subject will develop any cancer, or any combination of the cancers disclosed herein.

In other cases the method may be used to determine the risk that the subject will develop a cancer that expresses one or more specific TAAs. Suitable TAAs may be selected for use in the methods of the disclosure as further described below.

The terms “T cell response” and “immune response” are used herein interchangeably, and refer to the activation of T cells and/or the induction of one or more effector functions following recognition of one or more HLA-epitope binding pairs. In some cases an “immune response” includes an antibody response, because HLA class II molecules stimulate helper responses that are involved in inducing both long lasting CTL responses and antibody responses. Effector functions include cytotoxicity, cytokine production and proliferation.

The methods of the present disclosure may be used to determine an immunological risk of developing a cancer. Specifically the methods described herein may be used to determine a subject's ability to recognise and mount an immune response against TAAs or cancer cells that express those TAAs. Many other factors may contribute to a subject's overall risk of developing a cancer. Accordingly in some cases the methods disclosed herein may be combined with other risk determinants or incorporated into broader models for cancer risk prediction. For example a method of the present disclosure further comprises, in some embodiments, determining other cancer risk factors such as environmental factors, lifestyle factors, other genetic risk factors and any other factors that contribute to the subject's overall risk of developing cancer.

Not all the HLATs of a subject and/or that not all TAAs may play an equally important role in the immunological control of cancers. Therefore in some cases in accordance with the present disclosure a different weighting may be applied to different HLA alleles (for example using the “HLA-score” based method described in Examples 7 to 9 herein), to different HLAT, and/or to the HLAT that are capable of binding to the T cell epitopes of different TAAs (for example using the “HLAT-score” based method described in Examples 5 and 6 herein). The HLAT Score and HLA-score based methods exemplifying the invention differ in the technical computation, but in both cases a subject has a larger score if his/her predicted ability to generate immune response against TSAs is better. Both methods use a statistical learning algorithm. In case of the HLAT scores, the learning algorithm assigns weights to TSAs based on how important are the immune responses against them to fight against certain cancers. Then the final HLAT score is the weighted sums of HLA triplets that a subject can generate against the TSAs. In case of the HLA score, the learning algorithm assigns scores to individual HLA alleles based on how well HLATs can be generated against TSAs in a subject possessing that HLA allele. Then the final HLA score of a subject is the sum of the HLA alleles' weights he/she possesses.

In some cases the weighting to be applied may be determined empirically. For example in some cases the weighting applied to the HLAT that are capable of binding to the T cell epitope of a particular TAA may be determined by, based on or correlate to the capacity of each TAA to independently separate subjects having (the) cancer from subjects not having (the) cancer or from a background population of subjects including subjects having (the) cancer, using the methods described herein.

Alternatively or in addition the weighting applied to the HLAT that are capable of binding to the T cell epitope of a particular TAA may be determined by, based on, or correlate to frequency at which the TAA is expressed in a cancer or cancer type. Expression frequencies for TAAs in different cancers can be determined from published figures and scientific publications.

In some cases, the weighting applied to a particular HLAT may be determined by, based on, or correlate to the frequency with which the HLAT is present in subjects having cancer, or a subject and/or disease-matched subpopulation of subjects having cancer.

In some cases the weighting applied to the HLAT that are capable of binding to the T cell epitope of each TAA is defined as or using the following weight (w(c)):

$$w ( c ) = max \{ 0 , log ( \frac{0.05}{B} ) - log ( t ( c ) ) \}$$

where t(c) denotes the p-value of the one sided t-test on the HLAT score of the TAA c of the populations with and without cancer and B is the Bonferroni correction (number of TAAs). This weighting is used for the HLAT-score based method described herein.

In some cases the significance score (weighting) of an HLA allele (h) is defined as

$$s ( h ) \text{:=} sign ( h ) max \{ 0 , log ( \frac{0.05}{B} ) - log ( u ( h ) ) \}$$

where u(h) is the p-value of the two-sided u-test for allele h determining whether or not the number of HLATs are different in two subsets of individuals: one subset in which the individuals have HLA h, and one subset in which the individuals do not have HLA h. B is the Bonferroni correction, and sign(h) is +1 if the average number of HLATs is larger in the subpopulation having the h allele than in the subpopulation not having h, and −1 otherwise. This weighting is used for the HLA-score based method described herein.

In some cases, the initial weighting may be further optimised using any suitable method as known to those skilled in the art. In some cases the sum of these significance scores is used to determine the risk that the subject will develop cancer correlates to the risk that the subject will develop cancer.

For example, in some cases the risk that the subject will develop cancer correlates to or the risk that the subject will develop cancer is determined using the following HLAT Score (s(x)):

$$s ( x ) = {\sum }_{c ∈ C} w ( c ) p ( x , c )$$

where C is the set of the TAAs, c is a particular TAA, w(c) is the weight of TAA c, and p(x,c) is the HLAT number of the TAA c in subject x.

The HLAT Score based method and HLA-score based method described in the Examples herein are two examples of methods in accordance with the invention. Further scoring schemes can be developed by using the individuals' HLA class I genotype data. The concrete score to be used depends on the indication and the a priori data. In some cases, the choice will be made based on the performance of the different computations on available test datasets. The performance might be evaluated by the AUC value (the area under the ROC curve) or by any other goodness of performance score known by those skilled in the art.

### Tumor-Associated Antigens (TAAs)

Cancer- or tumor-associated antigen (TAAs) are proteins expressed in cancer or tumor cells. Examples of TAAs include new antigens (neoantigens, which are expressed during tumorigenesis and altered from the analogous protein in a normal or healthy cell), products of oncogenes and tumor suppressor genes, overexpressed or aberrantly expressed cellular proteins (e.g. HER2, MUC1), antigens produced by oncogenic viruses (e.g. EBV, HPV, HCV, HBV, HTLV), cancer testis antigens (CTA, e.g. MAGE family, NY-ESO), cell-type-specific differentiation antigens (e.g. MART-1) and Tumor Specific Antigen (TSA). A TSA is an antigen produced by a particular type of tumor that does not appear on normal cells of the tissue in which the tumor developed. TSAs include shared antigens, neoantigens, and unique antigens. TAA sequences may be found experimentally, or in published scientific papers, or through publicly available databases, such as the database of the Ludwig Institute for Cancer Research (cta.lncc.br/), Cancer Immunity database (cancerimmunity.org/peptide/) and the TANTIGEN Tumor T cell antigen database (cvc.dfci.harvard.edu/tadb/). Exemplary TAAs are listed in Tables 2 and 11.

TABLE 2
LIST OF NAMED TUMOUR ANTIGENS WITH CORRESPONDING ACCESSION NUMBERS.

| | | | |
|---|---|---|---|
| | 5T4 Q13641.1 | A1BG P04217.1 | A33 Q99795.1 |
| A4GALT Q9NPC4.1 | AACT P01011.1 | AAG Q9M6E9.1 | ABI1 Q8IZP0.1 |
| ABI2 Q9NYB9.1 | ABL1 P00519.1 | ABL-BCR Q8WUG5.1 | ABLIM3 O94929.1 |
| ABLL P42684.1 | ABTB1 Q969K4.1 | ACACA Q13085.1 | ACBD4 Q8NC06.1 |
| ACO1 P21399.1 | ACRBP Q8NEB7.1* | ACTL6A O96019.1 | ACTL8 Q9H568.1* |
| ACTN4 043707.1 | ACVR1 Q04771.1 | ACVR1B P36896.1 | ACVR2B Q13705.1 |
| ACVRL1 P37023.1 | ACS2B Q68CK6.1 | ACSL5 Q9ULC5.1 | ADAM-15 Q13444.1 |
| ADAM17 P78536.1 | ADAM2 Q99965.1* | ADAM29 Q9UKF5.1* | ADAM7 Q9H2U9.1 |
| ADAP1 O75689.1 | ADFP Q99541.1 | ADGRA3 Q8IWK6.1 | ADGRF1 Q5T601.1 |
| ADGRF2 Q8IZF7.1 | ADGRL2 O95490.1 | ADHFE1 Q8IWW8.1 | AEN Q8WTP8.1 |
| AFF1 P51825.1 | AFF4 Q9UHB7.1 | AFP P02771.1 | AGAP2 Q99490.1 |
| AGO1 Q9UL18.1 | AGO3 Q9H9G7.1 | AGO4 Q9HCK5.1 | AGR2 O95994.1 |
| AIFM2 Q9BRQ8.1 | AIM2 O14862.1 | AKAP-13 Q12802.1 | AKAP-3 O75969.1* |
| AKAP-4 Q5JQC9.1* | AKIP1 Q9NQ31.1 | AKT1 P31749.1 | AKT2 P31751.1 |
| AKT3 Q9Y243.1 | ALDH1A1 P00352.1 | ALK Q9UM73.1 | ALKBH1 Q13686.1 |
| ALPK1 Q96QP1.1 | AMIGO2 Q86SJ2.1 | ANG2 O15123.1 | ANKRD45 Q5TZF3.1* |
| ANO1 Q5XXA6.1 | ANP32A P39687.1 | ANXA2 P07355.1 | APC P25054.1 |
| APEH P13798.1 | APOA2 P02652.1 | APOD P05090.1 | APOL1 O14791.1 |
| AR P10275.1 | ARAF P10398.1 | ARF4L P49703.1 | ARHGEF5 Q12774.1 |
| ARID3A Q99856.1 | ARID4A P29374.1 | ARL6IP5 075915.1 | ARMC3 B4DXS3.1* |
| ARMC8 Q8IUR7.1 | ARTC1 P52961.1 | ARX Q96QS3.1* | ATAD2 Q6PL18.1 |
| ATIC P31939.1 | AURKC Q9UQB9.1 | AXIN1 O15169.1 | AXL P30530.1 |
| BAAT Q14032.1 | BAFF Q9Y275.1 | BAGE-1 Q13072.1* | BAGE-2 Q86Y30.1* |
| BAGE-3 Q86Y29.1* | BAGE-4 Q86Y28.1 | BAGE-5 Q86Y27.1* | BAI1 O14514.1 |
| BAL P19835.1 | BALF2 P03227.1 | BALF4 P03188.1 | BALF5 P03198.1 |
| BARF1 P03228.1 | BBRF1 P03213.1 | BCAN Q96GW7.1 | BCAP31 P51572.1 |
| BCL-2 P10415.1 | BCL2L1 Q07817.1 | BCL6 P41182.1 | BCL9 O00512.1 |
| BCR P11274.1 | BCRF1 P03180.1 | BDLF3 P03224.1 | BGLF4 P13288.1 |
| BHLF1 P03181.1 | BHRF1 P03182.1 | BILF1 P03208.1 | BILF2 P03218.1 |
| BIN1 O00499.1 | BING-4 O15213.1 | BIRC7 Q96CA5.1 | BLLF1 P03200.1 |
| BLLF2 P03199.1 | BMI1 P35226.1 | BMLF1 Q04360.1 | BMPR1B O00238.1 |
| BMRF1 P03191.1 | BNLF2a P0C739.1 | BNLF2b Q8AZJ3.1 | BNRF1 P03179.1 |
| BRAF1 P15056.1 | BRD4 O60885.1 | BRDT Q58F21.1* | BRI3BP Q8WY22.1 |
| BRINP1 O60477.1 | BRLF1 P03209.1 | BTBD2 Q9BX70.1 | BUB1B O60566.1 |
| BVRF2 P03234.1 | BXLF1 P03177.1 | BZLF1 P03206.1 | C15orf60 Q7Z4M0.1* |
| CA 12-5 Q8WXI7.1 | CA 19-9 Q969X2.1 | CA195 Q5TG92.1 | CA9 Q16790.1 |
| CABYR O75952.1* | CADM4 Q8NFZ8.1 | CAGE1 Q8CT20.1* | CALCA P01258.1 |
| CALR3 Q96L12.1 | CAN P35658.1 | CASC3 O15234.1 | CASC5 Q8NG31.1* |
| CASP5 P51878.1 | CASP8 Q14790.1 | CBFA2T2 O43439.1 | CBFA2T3 O75081.1 |
| CBL P22681.1 | CBLB Q13191.1 | CC3 Q9BUP3.1 | CCDC110 Q8TBZ0.1* |
| CCDC33 Q8N5R6.1* | CCDC36 Q8IYA8.1* | CCDC6 Q16204.1 | CCDC62 Q6P9F0.1* |
| CCDC68 Q9H2F9.1 | CCDC83 Q8IWF9.1* | CCL13 Q99616.1 | CCL2 P13500.1 |
| CCL7 P80098.1 | CCNA1 P78396.1* | CCNA2 P20248.1 | CCNB1 P14635.1 |
| CCND1 P24385.1 | CCNE2 O96020.1 | CCNI Q14094.1 | CCNL1 Q9UK58.1 |
| CCR2 P41597.1 | CD105 P17813.1 | CD123 P26951.1 | CD13 P15144.1 |
| CD133 O43490.1 | CD137 Q07011.1 | CD138 P18827.1 | CD157 Q10588.1 |
| CD16A P08637.1 | CD178 P48023.1 | CD19 P15391.1 | CD194 P51679.1 |
| CD2 P06729.1 | CD20 P11836.1 | CD21 P20023.1 | CD22 P20273.1 |
| CD229 Q9HBG7.1 | CD23 P06734.1 | CD27 P26842.1 | CD28 P10747.1 |
| CD30 P28908.1 | CD317 Q10589.1 | CD33 P20138.1 | CD350 Q9ULW2.1 |
| CD36 P16671.1 | CD37 P11049.1 | CD4 P01730.1 | CD40 P25942.1 |
| CD40L P29965.1 | CD45 P08575.1 | CD47 Q08722.1 | CD51 P06756.1 |
| CD52 P31358.1 | CD55 P08174.1 | CD61 P05106.1 | CD70 P32970.1 |
| CD74 P08922.1 | CD75 P15907.1 | CD79B P40259.1 | CD80 P33681.1 |
| CD86 P42081.1 | CD8a P01732.1 | CD8b P10966.1 | CD95 P25445.1 |
| CD98 P08195.1 | CDC123 O75794.1 | CDC2 P06493.1 | CDC27 P30260.1 |
| CDC73 Q6P1J9.1 | CDCA1 Q9BZD4.1* | CDCP1 Q9H5V8.1 | CDH3 P22223.1 |
| CDK2AP1 O14519.1 | CDK4 P11802.1 | CDK7 P50613.1 | CDKN1A P38936.1 |
| CDKN2A P42771.1 | CEA P06731.1 | CEACAM1 Q86UE4.1 | CENPK Q9BS16.1 |
| CEP162 Q5TB80.1 | CEP290 O15078.1* | CEP55 Q53EZ4.1* | CFL1 P23528.1 |
| CH3L2 Q15782.1 | CHEK1 O14757.1 | CK2 P19784.1 | CLCA2 Q9UQC9.1 |
| CLOCK O15516.1 | CLPP Q16740.1 | CMC4 P56277.1 | CML66 Q96RS6.1 |
| CO-029 P19075.1 | COTL1 Q14019.1 | COX2 P35354.1 | COX6B2 Q6YFQ2.1* |
| CPSF1 Q10570.1 | CPXCR1 Q8N123.1* | CREBL2 O60519.1 | CREG1 O75629.1 |
| Cripto P13385.1 | CRISP2 P16562.1* | *CRK P46108.1 | CRKL P46109.1 |
| CRLF2 Q9HC73.1 | CSAGE Q6PB30.1 | CT45 Q5HYN5.1* | CT45A2 Q5DJT8.1* |
| CT45A3 Q8NHU0.1* | CT45A4 Q8N7B7.1* | CT45A5 Q6NSH3.1* | CT45A6 P0DMU7.1* |
| CT46 Q86X24.1* | CT47 Q5JQC4.1* | CT47B1 P0C2P7.1* | CTAGE2 Q96RT6.1* |
| cTAGE5 O15320.1* | CTCFL Q8NI51.1* | CTDSP2 O14595.1 | CTGF P29279.1 |
| CTLA4 P16410.1 | CTNNA2 P26232.1* | CTNNB1 P35222.1 | CTNND1 O60716.1 |
| CTSH P09668.1 | CTSP1 A0RZH4.1* | CTTN Q14247.1 | CXCR4 P61073.1 |
| CXorf48 Q8WUE5.1* | CXorf61 Q5H943.1* | Cyclin-E P24864.1 | CYP1B1 Q16678.1 |
| CypB P23284.1 | CYR61 O00622.1 | CS1 P28290.1 | CSAG1 Q6PB30.1* |
| CSDE1 O75534.1 | CSF1 P09603.1 | CSF1R P07333.1 | CSF3R Q99062.1 |
| CSK P41240.1 | CSK23 Q8NEV1.1 | DAPK3 O43293.1 | DAZ1 Q9NQZ3.1 |
| DBPC Q9Y2T7.1 | DCAF12 Q5T6F0.1* | DCT P40126.1 | DCUN1D1 Q96GG9.1 |
| DCUN1D3 Q8IWE4.1 | DDR1 Q08345.1 | DDX3X O00571.1 | DDX6 P26196.1 |
| DEDD O75618.1 | DEK P35659.1 | DENR O43583.1 | DEPDC1 Q5TB30.1 |
| DFNA5 O60443.1 | DGAT2 Q96PD7.1 | DHFR P00374.1 | DKK1 O94907.1 |
| DKK3 Q9UBP4.1 | DKKL1 Q9UK85.1* | DLEU1 O43261.1 | DMBT1 Q9UGM3.1 |
| DMRT1 Q9Y5R6.1* | DNAJB8 Q8NHS0.1* | DNAJC8 O75937.1 | DNMT3A Q9Y6K1.1 |
| DPPA2 Q7Z7J5.1* | DR4 O00220.1 | DR5 O14763.1 | DRG1 Q9Y295.1* |
| DSCR8 Q96T75.1 | E2F3 O00716.1 | E2F6 O75461.1 | E2F8 A0AVK6.1 |
| EBNA1 P03211.1 | EBNA2 P12978.1 | EBNA3 P12977.1 | EBNA4 P03203.1 |
| EBNA6 P03204.1 | EBNA-LP Q8AZK7.1 | E-cadherin P12830.1 | ECT2 Q9H8V3.1 |
| ECTL2 Q008S8.1 | EDAG Q9BXL5.1* | EEF2 P13639.1 | EFNA1 P20827.1 |
| EFS O43281.1 | EFTUD2 Q15029.1 | EGFL7 Q9UHF1.1 | EGFR p00533.1 |
| EI24 O14681.1 | EIF4EBP1 Q13541.1 | ELF3 P78545.1 | ELF4 Q99607.1 |
| ELOVL4 Q9GZR5.1* | EMP1 P54849.1 | ENAH Q8N8S7.1 | Endosialin Q9HCU0.1 |
| ENO1 P06733.1 | ENO2 P09104.1 | EN03 P13929.1 | ENTPD5 O75356.1 |
| EpCAM P16422.1 | EPHA2 P29317.1 | EPHA3 P29320.1 | EPHB2 P29323.1 |
| EPHB4 P54760.1 | EPHB6 O15197.1 | EPS8 Q12929.1 | ERBB3 P21860.1 |
| ERBB4 Q15303.1 | EREG O14944.1 | ERG P11308.1 | ERVK-18 O42043.1 |
| ERVK-19 O71037.1 | ESR1 P03372.1 | ETAA1 Q9NY74.1 | ETS1 P14921.1 |
| ETS2 P15036.1 | ETV1 P50549.1 | ETV5 P41161.1 | ETV6 P41212.1 |
| EVI5 O60447.1 | EWSR1 Q01844.1 | EYA2 O00167.1 | EZH2 Q15910.1 |
| FABP7 O15540.1 | FAM133A Q8N9E0.1* | FAM13A O94988.1 | FAM46D Q8NEK8.1* |
| FAM58BP P0C7Q3.1 | FANCG O15287.1 | FATE1 Q969F0.1* | FBXO39 Q8N4B4.1* |
| FBXW11 Q9UKB1.1 | FCHSD2 O94868.1 | FER P16591.1 | FES P07332.1 |
| FEV Q99581.1 | FGF10 O15520.1 | FGF23 Q9GZV9.1 | FGF3 P11487.1 |
| FGF4 P08620.1 | FGF5 P12034.1 | FGFR1 P11362.1 | FGFR2 P21802.1 |
| FGFR3 P22607.1 | FGFR4 P22455.1 | FGR P09769.1 | FLU Q01543.1 |
| FLT3 P36888.1 | FMNL1 O95466.1 | FMOD Q06828.1 | FMR1NB Q8N0W7.1 * |
| FN1 P02751.1 | Fn14 Q9NP84.1 | FNIP2 Q9P278.1 | FOLR1 P15328.1 |
| FOS P01100.1 | FosB P53539.1 | FOSL1 P15407.1 | FOXM1 Q08050.1 |
| FOXO1 Q12778.1 | FOXO3 O43524.1 | FRAT1 Q92837.1 | FRMD3 A2A2Y4.1 |
| FSIP1 Q8NA03.1 | FSIP2 Q5CZC0.1 | FSTL3 O95633.1 | FTHL17 Q9BXU8.1* |
| FUNDC2 Q9BWH2.1 | FUS P35637.1 | FUT1 P19526.1 | FUT3 P21217.1 |
| FYN P06241.1 | GAB2 Q9UQC2.1 | GADD45G O95257.1 | GAGE-1 Q13065.1 |
| GAGE12B/C/D/E A1L429.1 | GAGE12F P0CL80.1 | GAGE12G P0CL81.1 | GAGE12H A6NDE8.1 |
| GAGE12I P0CL82.1 | GAGE12J A6NER3.1 | GAGE-2 Q6NT46.1 | GAGE-3 Q13067.1 |
| GAGE-4 Q13068.1 | GAGE-5 Q13069.1 | GAGE-6 Q13070.1 | GAGE-7 O76087.1 |
| GAGE-8 Q9UEU5.1 | GALGT2 Q00973.1 | GAS7 O60861.1 | GASZ Q8WWH4.1 |
| GATA-3 P23771.1 | GBU4-5 Q587J7.1 | GCDFP-15 P12273.1 | GFAP P14136.1 |
| GFI1 Q99684.1 | Ghrelin Q9UBU3.1 | GHSR Q92847.1 | GIPC1 O14908.1 |
| GITR Q9Y5U5.1 | GKAP1 Q5VSY0.1 | GLI1 P08151.1 | Glypican-3 P51654.1 |
| GML Q99445.1 | GNA11 P29992.1 | GNAQ P50148.1 | GNB2L1 P63244.1 |
| GOLGA5 Q8TBA6.1 | gp100 P40967.1 | gp75 P17643.1 | Gp96 P14625.1 |
| GPAT2 Q6NUI2.1* | GPATCH2 Q9NW75.1* | GPC-3 P51654.1 | GPNMB Q14956.1 |
| GPR143 P51810.1 | GPR89A B7ZAQ6.1 | GRB2 P62993.1 | GRP78 P11021.1 |
| GUCY1A3 Q02108.1 | H3F3A P84243.1 | HAGE Q9NXZ2.1* | hANP P01160.1 |
| HBEGF Q99075.1 | hCG-beta P01233.1 | HDAC1 Q13547.1 | HDAC2 Q92769.1 |
| HDAC3 O15379.1 | HDAC4 P56524.1 | HDAC5 Q9UQL6.1 | HDAC6 Q9UBN7.1 |
| HDAC7 Q8WUI4.1 | HDAC8 Q9BY41.1 | HDAC9 Q9UKV0.1 | HEATR1 Q9H583.1 |
| Hepsin P05981.1 | Her2/neu P04626.1 | HERC2 O95714.1 | HERV-K104 P61576.1 |
| HEXB P07686.1 | HEXIM1 O94992.1 | HGRG8 Q9Y5A9.1 | HIPK2 Q9H2X6.1 |
| HJURP Q8NCD3.1 | HMGB1 P09429.1 | HMOX1 P09601.1 | HNRPL P14866.1 |
| HOM-TES-85 Q9P127.1* | HORMAD1 Q86X24.1* | HORMAD2 Q8N7B1.1* | HPSE Q9Y251.1 |
| HPV16 E6 P03126.1 | HPV16 E7 P03129.1 | HPV18 E6 P06463.1 | HPV18 E7 P06788.1 |
| HRAS P01112.1 | HSD17B13 Q7Z5P4.1 | HSP105 Q92598.1 | HSP60 P10809.1 |
| HSPA1A P08107.1 | HSPB9 Q9BQS6.1* | HST-2 P10767.1 | HT001 Q2TB18.1 |
| hTERT O14746.1 | HUS1 O60921.1 | ICAM-1 P05362.1 | IDH1 O75874.1 |
| IDO1 P14902.1 | IER3 P46695.1 | IGF1R P08069.1 | IGFS11 Q5DX21.1* |
| IL13RA2 Q14627.1* | IMP-3 Q9NV31.1* | ING3 Q9NXR8.1 | INPPL1 O15357.1 |
| INTS6 Q9UL03.1 | IRF4 Q15306.1 | IRS4 O14654.1 | ITGA5 P08648.1 |
| ITGB8 P26012.1 | ITPA Q9BY32.1 | ITPR2 Q14571.1 | JAK2 O60674.1 |
| JAK3 P52333.1 | JARID1B Q9UGL1.1* | JAZF1 Q86VZ6.1 | JNK1 P45983.1 |
| JNK2 P45984.1 | JNK3 P53779.1 | JTB O76095.1 | JUN P05412.1 |
| JUP P14923.1 | K19 P08727.1 | KAAG1 Q9UBP8.1 | Kallikrein 14 Q9P0G3.1 |
| Kallikrein 4 Q9Y5K2.1 | KAT6A Q92794.1 | KDM1A O60341.1 | KDM5A P29375.1 |
| KIAA0100 Q14667.1* | KIAA0336 Q8IWJ2.1 | KIAA1199 Q8WUJ3.1 | KIAA1641 A6QL64.1 |
| KIF11 P52732.1 | KIF1B O60333.1 | KIF20A O95235.1 | KIT P10721.1 |
| KLF4 O43474.1 | KLHL41 O60662.1 | KLK10 O43240.1 | KMT2D O14686.1 |
| KOC1 O00425.1 | K-ras P01116.1 | KRIT1 O00522.1 | KW-12 P62913.1 |
| KW-2 Q96RS0.1 | KW-5 (SEBD4) Q9H0Z9.1 | KW-7 O75475.1 | L1CAM P32004.1 |
| L53 Q96EL3.1 | L6 Q9BTT4.1 | LAG3 P18627.1 | Lage-1 O75638.1* |
| LATS1 O95835.1 | LATS2 Q9NRM7.1 | LCMT2 O60294.1 | LCP1 P13796.1 |
| LDHC P07864.1* | LDLR P01130.1 | LEMD1 Q68G75.1* | Lengsin Q5TDP6.1 |
| LETMD1 Q6P1Q0.1 | LGALS3BP Q08380.1 | LGALS8 O00214.1 | LIN7A O14910.1 |
| LIPI Q6XZB0.1* | LIV-1 Q13433.1 | LLGL1 Q15334.1 | LMO1 P25800.1 |
| LMO2 P25791.1 | LMP1 P03230.1 | LMP2 P13285.1 | LOC647107 Q8TAI5.1* |
| L0XL2 Q9Y4K0.1 | LRP1 Q07954.1 | LRRN2 O75325.1 | LTF P02788.1 |
| LTK P29376.1 | LZTS1 Q9Y250.1 | LY6K Q17RY6.1* | LYN P07948.1 |
| LYPD6B Q8NI32.1* | MAEA Q7L5Y9.1 | MAEL Q96JY0.1* | MAF O75444.1 |
| MAFF Q9ULX9.1 | MAFG O15525.1 | MAFK O60675.1 | MAGE-A1 P43355.1* |
| MAGE-A10 P43363.1* | MAGE-A11 P43364.1* | MAGE-A12 P43365.1* | MAGE-A2 P43356.1* |
| MAGE-A2B Q6P448.1* | MAGE-A3 P43357.1* | MAGE-A4 P43358.1* | MAGE-A5 P43359.1* |
| MAGE-A6 P43360.1* | MAGE-A8 P43361.1* | MAGE-A9 P43362.1* | MAGE-B1 P43366.1* |
| MAGE-B2 015479.1* | MAGE-B3 O15480.1* | MAGE-B4 O15481.1* | MAGE-B5 Q9BZ81.1* |
| MAGE-B6 Q8N7X4.1* | MAGE-C1 O60732.1* | MAGE-C2 Q9UBF1.1* | MAGE-C3 Q8TD91.1* |
| mammaglobin-A Q13296.1 | MANF P55145.1 | MAP2K2 P36507.1 | MAP2K7 O14733.1 |
| MAP3K7 O43318.1 | MAP4K5 Q9Y4K4.1 | MART1 Q16655.1 | MART-2 Q5VTY9.1 |
| MAS1 P04201.1 | MC1R Q01726.1 | MCAK Q99661.1* | MCF2 P10911.1 |
| MCF2L O15068.1 | MCL1 Q07820.1 | MCTS1 Q9ULC4.1 | MCSP Q6UVK1.1 |
| MDK P21741.1 | MDM2 Q00987.1 | MDM4 O15151.1 | ME1 P48163.1 |
| ME491 P08962.1 | MECOM Q03112.1 | MELK Q14680.1 | MEN1 O00255.1 |
| MERTK Q12866.1 | MET P08581.1 | MFGE8 Q08431.1 | MFHAS1 Q9Y4C4.1 |
| MFI2 P08582.1 | MGAT5 Q09328.1 | Midkine P21741.1 | MIF P14174.1 |
| MKI67 P46013.1 | MLH1 P40692.1 | MLL Q03164.1 | MLLT1 Q03111.1 |
| MLLT10 P55197.1 | MLLT11 Q13015.1 | MLLT3 P42568.1 | MLLT4 P55196.1 |
| MLLT6 P55198.1 | MMP14 P50281.1 | MMP2 P08253.1 | MMP7 P09237.1 |
| MMP9 P14780.1 | MOB3B Q86TA1.1 | MORC1 Q86VD1.1* | MPHOSPH1 Q96Q89.1* |
| MPL P40238.1 | MRAS O14807.1 | MRP1 P33527.1 | MRP3 O15438.1 |
| MRPL28 Q13084.1 | MRPL30 Q8TCC3.1 | MRPS11 P82912.1 | MSLN Q13421.1 |
| MTA1 Q13330.1 | MTA2 O94776.1 | MTA3 Q9BTC8.1 | MTCP1 P56278.1 |
| MTSS1 O43312.1 | MUC-1 P15941.1 | MUC-2 Q02817.1 | MUC-3 Q02505.1 |
| MUC-4 Q99102.1 | MUC-5AC P98088.1 | MUC-6 Q6W4X9.1 | MUM1 Q2TAK8.1 |
| MUM2 Q9Y5R8.1 | MYB P10242.1 | MYC P01106.1 | MYCL P12524.1 |
| MYCLP1 P12525.1 | MYCN P04198.1 | MYD88 Q99836.1 | MYEOV Q96EZ4.1 |
| MY01B O43795.1 | NA88-A P0C5K6.1* | NAE1 Q13564.1 | Napsin-A O96009.1 |
| NAT6 Q93015.1 | NBAS A2RRP1.1 | NBPF12 Q5TAG4.1 | NCOA4 Q13772.1 |
| NDC80 O14777.1 | NDUFC2 O95298.1 | Nectin-4 Q96NY8.1 | NEK2 P51955.1 |
| NEMF O60524.1 | NENF Q9UMX5.1 | NEURL1 O76050.1 | NFIB O00712.1 |
| NFKB2 Q00653.1 | NF-X1 Q12986.1 | NFYC Q13952.1 | NGAL P80188.1 |
| NGEP Q6IWH7.1 | NKG2D-L1 Q9BZM6.1 | NKG2D-L2 Q9BZM5.1 | NKG2D-L3 Q9BZM4.1 |
| NKG2D-L4 Q8TD07.1 | NKX3.1 Q99801.1 | NLGN4X Q8N0W4.1 | NLRP4 Q96MN2.1* |
| NNMT P40261.1 | NOL4 O94818.1* | N0TCH2 Q04721.1 | NOTCH3 Q9UM47.1 |
| N0TCH4 Q99466.1 | NOV P48745.1 | NPM1 P06748.1 | NR6A1 Q15406.1* |
| N-RAS P01111.1 | NRCAM Q92823.1 | NRP1 O14786.1 | NSE1 Q96KN4.1 |
| NSE2 Q96KN1.1 | NTRK1 P04629.1 | NUAK1 O60285.1 | NUGGC Q68CJ6.1 |
| NXF2 Q9GZY0.1* | NXF2B Q5JRM6.1* | NY-BR-1 Q9BXX3.1 | NYD-TSPG Q9BWV7.1 |
| NY-ESO-1 P78358.1* | NY-MEL-1 P57729.1 | OCA2 Q04671.1 | ODF1 Q14990.1* |
| ODF2 Q5BJF6.1* | ODF3 Q96PU9.1* | ODF4 Q2M2E3.1* | OGG1 O15527.1 |
| OGT O15294.1 | OIP5 O43482.1* | OS9 Q13438.1 | OTOA Q05BM7.1* |
| OX40 P43489.1 | OX40L P23510.1 | P53 P04637.1 | P56-LCK P06239.1 |
| PA2G4 Q9UQ80.1 | PAGE1 O75459.1* | PAGE2 Q7Z2X2.1* | PAGE2B Q5JRK9.1* |
| PAGE3 Q5JUK9.1* | PAGE4 O60829.1* | PAGE5 Q96GU1.1* | PAK2 Q13177.1 |
| PANO1 I0J062.1 | PAP Q06141.1 | PAPOLG Q9BWT3.1 | PARK2 O60260.1 |
| PARK7 Q99497.1 | PARP12 Q9H0J9.1 | PASD1 Q8IV76.1* | PAX3 P23760.1 |
| PAX5 Q02548.1 | PBF P00751.1 | PBK Q96KB5.1* | PBX1 P40424.1 |
| PCDC1 Q15116.1 | PCM1 Q15154.1 | PCNXL2 A6NKB5.1 | PDGFB P01127.1 |
| PDGFRA P16234.1 | PEPP2 Q9HAU0.1* | PGF P49763.1 | PGK1 P00558.1 |
| PHLDA3 Q9Y5J5.1 | PHLPP1 O60346.1 | PIAS1 O75925.1 | PIAS2 O75928.1 |
| PIK3CA P42336.1 | PIK3CD O00329.1 | PIK3R2 O00459.1 | PIM1 P11309.1 |
| PIM2 Q9P1W9.1 | PIM3 Q86V86.1 | PIR O0065.1 | PIWIL1 Q96J94.1* |
| PIWIL2 Q8TC59.1* | PIWIL3 Q7Z3Z3.1 | PIWIL4 Q7Z3Z4.1 | PKN3 Q6P5Z2.1 |
| PLA2G16 P53816.1 | PLAC1 Q9HBJ0.1* | PLAG1 Q6DJT9.1 | PLEKHG5 O94827.1 |
| PLK3 Q9H4B4.1 | PLS3 P13797.1 | PLVAP Q9BX97.1 | PLXNB1 O43157.1 |
| PLXNB2 O15031.1 | PML P29590.1 | PML-RARA Q96QH2.1 | POYEA Q6S8J7.1* |
| POTEB Q6S5H4.1* | POTEC B2RU33.1* | POTED Q86YR6.1* | POYEE Q6S8J3.1* |
| POTEG Q6S5H5.1* | POTEH Q6S545.1* | PP2A P63151.1 | PPAPDC1B Q8NEB5.1 |
| PPFIA1 Q13136.1 | PPIG Q13427.1 | PPP2R1B P30154.1 | PRAME P78395.1* |
| PRDX5 P30044.1 | PRKAA1 Q13131.1 | PRKCI P41743.1 | PRM1 P04553.1* |
| PRM2 P04554.1* | PRMT3 O60678.1 | PRMT6 Q96LA8.1 | PDL1 Q9NZQ7.1 |
| PROM1 O43490.1 | PRSS54 Q6PEW0.1* | PRSS55 Q6UWB4.1* | PRTN3 P24158.1 |
| PRUNE Q86TP1.1 | PRUNE2 Q8WUY3.1 | PSA P07288.1 | PSCA D3DWI6.1 |
| PSMA Q04609.1 | PSMD10 O75832.1 | PSGR Q9H255.1 | PSP-94 Q1L6U9.1 |
| PTEN P60484.1 | PTH-rP P12272.1 | PTK6 Q13882.1 | PTPN20A Q4JDL3.1* |
| PTPRK Q15262.1 | PTPRZ P23471.1 | PTTG-1 O95997.1 | PTTG2 Q9NZH5.1 |
| PTTG3 Q9NZH4.1 | PXDNL A1KZ92.1 | RAB11FIP3 O75154.1 | RAB8A P61006.1 |
| RAD1 O60671.1 | RAD17 O75943.1 | RAD51C O43502.1 | RAFI P04049.1 |
| RAGE-1 Q9UQ07.1 | RAP1A P62834.1 | RARA P10276.1 | RASSF10 A6NK89.1 |
| RB1 P06400.1 | RBL2 Q08999.1 | RBM46 Q8TBY0.1* | RBP4 P02753.1 |
| RCAS1 O00559.1 | RCVRN P35243.1 | RECQL4 O94761.1 | RET P07949.1 |
| RGS22 Q8NE09.1* | RGS5 O15539.1 | RHAMM O75330.1 | RhoC P08134.1 |
| RHOXF2 Q9BQY4.1 | RL31 P62888.1 | RNASET2 O00584.1 | RNF43 Q68DV7.1 |
| RNF8 O76064.1 | RON Q04912.1 | ROPN1A Q9HAT0.1* | ROR1 Q01973.1 |
| RPA1 O95602.1 | RPL10A P62906.1 | RPL7A P62424.1 | RPS2 P15880.1 |
| RPS6KA5 O75582.1 | RPSA P08865.1 | RQCD1 Q92600.1* | RRAS2 P62070.1 |
| RSL1D1 O76021.1 | RTKN Q9BST9.1 | RUNX1 Q01196.1 | RUNX2 Q13950.1 |
| RYK P34925.1 | SAGE1 Q9NXZ1.1* | SART2 Q9UL01.1 | SART3 Q15020.1 |
| SASH1 O94885.1 | sCLU P10909.1 | SCRN1 Q12765.1 | SDCBP O00560.1 |
| SDF-1 P48061.1 | SDHD O14521.1 | SEC31A O94979.1 | SEC63 Q9UGP8.1 |
| Semaphorin 4D Q92854.1 | SEMG1 P04279.1* | SFN P31947.1 | SH2B2 O14492.1 |
| SH2D1B O14796.1 | SH3BP1 Q9Y3L3.1 | SHB Q15464.1 | SHC3 Q92529.1 |
| SIRT2 Q8IXJ6.1 | SIVA1 O15304.1 | SKI P12755.1 | SLBP A9UHW6.1 |
| SLC22A10 Q63ZE4.1 | SLC25A47 Q6Q0C1.1 | SLC35A4 Q96G79.1 | SLC45A3 Q96JT2.1 |
| SLC4A1AP Q9BWU0.1 | SLC06A1 Q86UG4.1* | SLITRK6 Q9H5Y7.1 | Sm23 P27701.1 |
| SMAD5 Q99717.1 | SMAD6 O43541.1 | SMO Q99835.1 | Smt3B P61956.1 |
| SNRPD1 P62314.1 | SOS1 Q07889.1 | SOX-2 P48431.1 | SOX-6 P35712.1 |
| SOX-11 P35716 .1 | SPA17 Q15506.1* | SPACA3 Q8IXA5.1* | SPAG1 Q07617.1* |
| SPAG17 Q6Q759.1* | SPAG4 Q9NPE6.1* | SPAG6 O75602.1* | SPAG8 Q99932.1* |
| SPAG9 O60271.1* | SPANXA1 Q9NS26.1* | SPANXB Q9NS25.1* | SPANXC Q9NY87.1* |
| SPANXD Q9BXN6.1* | SPANXE Q8TAD1.1* | SPANXN1 Q5VSR9.1* | SPANXN2 Q5MJ10.1* |
| SPANXN3 Q5MJ09.1* | SPANXN4 Q5MJ08.1* | SPANXN5 Q5MJ07.1* | SPATA19 Q7Z5L4.1* |
| SPEF2 Q9C093.1* | SPI1 P17947.1 | SPINLW1 O95925.1* | SPO11 Q9Y5K1.1* |
| SRC P12931.1 | SSPN Q14714.1 | SSX-1 Q16384.1* | SSX-2 Q16385.1* |
| SSX-3 Q99909.1* | SSX-4 O60224.1* | SSX-5 060225.1* | SSX-6 Q7RTT6.1* |
| SSX-7 Q7RTT5.1* | SSX-9 Q7RTT3.1* | ST18 O60284.1 | STAT1 P42224.1 |
| STEAP1 Q9UHE8.1 | STK11 Q15831.1 | STK25 O00506.1 | STK3 Q13188.1 |
| STN Q9H668.1 | SUPT7L O94864.1 | Survivin O15392.1 | SUV39H1 O43463.1 |
| SYCE1 Q8N0S2.1 | SYCP1 Q15431.1 | SYCP3 Q8IZU3.1 | SYT Q15532.1 |
| TA-4 Q96RI8.1 | TACC1 O75410.1 | TAF1B Q53T94.1 | TAF4 O00268.1 |
| TAF7L Q5H9L4.1* | TAG-1 Q02246.1* | TAL1 P17542.1 | TAL2 Q16559.1 |
| TAPBP O15533.1 | TATI P00995.1 | TAX1BP3 O14907.1 | TBC1D3 Q8IZP1.1 |
| TBP-1 P17980.1 | TCL1A P56279.1 | TCL1B O95988.1 | TDHP Q9BT92.1 |
| TDRD1 Q9BXT4.1* | TDRD4 Q9BXT8.1* | TDRD6 O60522.1* | TEKT5 Q96M29.1* |
| TEX101 Q9BY14.1* | TEX14 Q8IWB6.1* | TEX15 Q9BXT5.1* | TEX38 Q6PEX7.1* |
| TF P02787.1 | TFDP3 Q5H9I0.1* | TFE3 P19532.1 | TGFBR1 P36897.1 |
| TGFBR2 P37173.1 | THEG Q9P2T0.1* | TIE2 Q02763.1 | TIPRL O75663.1 |
| TLR2 O60603.1 | TMEFF1 Q8IYR6.1* | TMEFF2 Q9UIK5.1* | TMEM108 Q6UXF1.1* |
| TMEM127 O75204.1 | TMPRSS12 Q86WS5.1* | TNC P24821.1 | TNFRSF17 Q02223.1 |
| TNFSF15 O95150.1 | TNK2 Q07912.1 | TOMM34 Q15785.1 | TOP2A P11388.1 |
| TOP2B Q02880.1 | TOR3A Q9H497.1 | TP73 O15350.1 | TPA1 8N543.1 |
| TPGS2 Q68CL5.1 | TPI1 P60174.1 | TPL2 P41279.1 | TPM4 P67936.1 |
| TPO P40225.1 | TPPP2 P59282.1* | TPR P12270.1 | TPTE P56180.1* |
| TRAF5 O00463.1 | TRAG-3 Q9Y5P2.1* | TRGC2 P03986.1 | TRIM24 O15164.1 |
| TRIM37 O94972.1 | TRIM68 Q6AZZ1.1 | TRPM8 Q7Z2W7.1 | TSGA10 Q9BZW7.1* |
| TSP50 Q9UI38.1* | TSPAN6 O43657.1 | TSPY1 Q01534.1* | TSPY2 A6NKD2.1* |
| TSPY3 Q6B019.1* | TSPYL1 Q9H0U9.1 | TSSK6 Q9BXA6.1* | TTC23 Q5W5X9.1 |
| TTK P33981.1* | TULP2 O00295.1* | TUSC2 O75896.1 | TWEAK O43508.1 |
| TXNIP Q9H3M7.1 | TYMS P04818.1 | TYR P14679.1 | U2 snRNP B P08579.1 |
| U2AF1 Q01081.1 | UBD O15205.1 | UBE2A P49459.1 | UBE2C O00762.1 |
| UBE2V1 Q13404.1 | UBE4B O95155.1 | UBR5 O95071.1 | UBXD5 Q5T124.1 |
| UFL1 O94874.1 | URI1 O94763.1 | URLC10 Q17RY6.1 | UROC1 Q96N76.1 |
| USP2 O75604.1 | USP4 Q13107.1 | VAV1 P15498.1 | VCX3A Q9NNX9.1 |
| VEGFR1 P17948.1 | VEGFR2 P35968.1 | VHL P40337.1 | VIM P08670.1 |
| VWA5A O00534.1 | WHSC2 Q9H3P2.1 | WISP1 O95388.1 | WNK2 Q9Y3S1.1 |
| WNT10B O00744.1 | WNT3 P56703.1 | WNT-5a P41221.1 | WT1 P19544.1 |
| WWP1 Q9H0M0.1 | XAGE-1 Q9HD64.1* | XAGE-2 Q96GT9.1* | XAGE-3 Q8WTP9.1* |
| XAGE-4 Q8WWM0.1 | XAGE-5 Q8WWM1.1* | XBP1 P17861.1 | XPO1 O14980.1 |
| XRCC3 O43542.1 | YB-1 P67309.1 | YEATS4 O95619.1 | YES1 P07947.1 |
| YKL-40 P36222.1 | ZBTB7A O95365.1 | ZBTB7C A1YPR0.1 | ZEB1 P37275.1 |
| ZFYVE19 Q96K21.1 | ZNF165 P49910.1* | ZNF185 O15231.1 | ZNF217 O75362.1 |
| ZNF320 A2RRD8.1 | ZNF395 Q9H3N7.1 | ZNF645 Q8N7E2.1* | ZUBR1 Q5T4S7.1 |
| ZW10 O43264.1 | ZWINT O95229.1 | Ropporin-1A Q9HAT0 | WBP2NL Q6ICG8.1 |

TSAs/CTAs = bold and *
Table 2 optionally excludes Ropporin-1A Q9HAT0 and/or WBP2NL Q6ICG8.1.

In some cases the methods described herein are used to determine the risk that a subject will develop a cancer that expresses one or more specific TAAs. In other cases the method is used to determine the risk that that a subject will develop any cancer or a particular type of cancer. Different TAAs may in some cases be associated with different types of cancer, but not every cancer of a particular type will express the same combination of TAAs. Therefore in some cases the epitope-binding HLAT is quantified in multiple TAAs, in some cases at least 2, 3, 4, 5, 6, 7, 8, 9, 10, 12, 15, 20, 25, 30, 35, 40, 45 or more TAA. In general fewer TAAs may be used if the TAAs are expressed in a higher proportion of cancers or cancer patients or cancers of a selected type. More TAAs may be used if the TAAs are expressed in a lower proportion of cancers or cancer patients or cancers of a selected type. In some cases a set of TAAs may be used that together are expressed or over-expressed in a minimum proportion of cancers, cancer patients, or cancers of a selected type, for example 50%, 55%, 60%, 65%, 70%, 75%, 80%, 85%, 90%, 95%, 98% or more. Expression frequencies for TAAs in different cancers can be determined from published figures and scientific publications.

A TAA selected for use in accordance with the present disclosure is typically one that is expressed or over-expressed in a high proportion of cancers or cancers of a particular type. In some cases one or more or each of the TAAs may be expressed or over-expressed in at least 1%, 2%, 5%, 10%, 15%, 20%, 25%, 30%, 35%, 40%, 45%, 50%, 55%, 60%, 65%, 70%, 75%, 80%, 85%, 90%, 95% or the cancers, or in the cancers of a disease and/or subject-matched human population. For example the subject may be matched by ethnicity, geographical location, gender, age, disease, disease type or stage, genotype, the expression of one or more biomarkers or the like, or any combination thereof.

In some cases one or more or each of the TAAs is a tumor specific antigen (TSA) or a cancer testis antigens (CTA). CTA are not typically expressed beyond embryonic development in healthy cells. In healthy adults, CTA expression is limited to male germ cells that do not express HLAs and cannot present antigens to T cells. Therefore, CTAs are considered expressional neoantigens when expressed in cancer cells. CTA expression is (i) specific for tumor cells, (ii) more frequent in metastases than in primary tumors and (iii) conserved among metastases of the same patient (Gajewski ed. Targeted Therapeutics in Melanoma. Springer New York. 2012).

In some cases the method comprises the step of selecting and/or identifying suitable TAAs or a suitable set of TAAs for use in the method disclosed herein.

### Methods of Treatment

In some cases the methods described herein comprise the selection, preparation and/or administration of a treatment for a cancer in a subject. The subject may have been determined to have an elevated risk of developing the cancer using a method as described herein. A “treatment” as used herein is any action taken to prevent or delay the onset of cancer, to ameliorate one or more symptom or complication, to induce or prolong remission, to delay a relapse, recurrence or deterioration, or otherwise improve or stabilise the disease status of or cancer risk to the subject. Typically the treatment will be a prophylactic treatment intended to delay or prevent onset of cancer or any symptom or complication associated with cancer. The treatment may be immunotherapy or vaccination.

The term “treatment” as used herein may in some cases encompass recommendations concerning the behaviour, environmental exposure or lifestyle of the subject that are intended to reduce the risk that the subject will develop cancer or any symptom or complication associated with the cancer. For example, for a subject that is determined to have an elevated risk of developing melanoma the treatment may include recommending a reduction in exposure of the subject to UV radiation. This may, for example, include avoiding artificial UV sources, reducing sun exposure or avoiding sun exposure at certain times of the day, applying sunscreen that provides suitable protection, wearing protective clothing, avoiding burning, and/or taking vitamin D. In other example the treatment may include recommendations related to diet, including the use of dietary supplements (for example anti-oxidant supplements, or increased calcium intake), drug use (including reducing tobacco and/or alcohol consumption), exercise, or exposure to potential carcinogens, infectious agents and/or radiation.

In other cases the treatment may include additional or increased frequency of screenings or examinations intended to achieve early diagnosis of cancer. In other cases the treatment may include the administration of anti-inflammatory medications, such as aspirin or non-steroidal anti-inflammatory drugs, or avoiding or reducing the administration of immunosuppressive drugs. In some cases the treatment may include increased attention to the management of other conditions that are potential risk factors, such as obesity, or conditions that are associated with chronic inflammation such as ulcerative colitis and Crohn's disease.

In other cases the treatment may be any known therapeutic or prophylactic treatment for cancer, such as surgery, chemotherapy, cytotoxic or non-cytotoxic chemotherapy, radiation therapy, targeted therapy, hormone therapy, or the administration of targeted small-molecule drugs or antibodies, e.g. monoclonal antibodies or co-stimulatory antibodies and including any cancer treatment described herein.

Treatments that are intended to enhance a subject's immune response to cancer cells are likely to be particularly effective in preventing or delaying the development of cancer in a subject that is determined to have an elevated risk of cancer using a method described herein. Accordingly in some cases the treatment may be immunotherapy or checkpoint blockade therapy or checkpoint inhibitor therapy. In some cases the method comprises administering to the subject one or more peptides or one of more polynucleic acids or vectors that encode one or more peptides as described below, that comprise an amino acid sequence that is (i) a fragment of an antigen that is associated with expression in the cancer; and (ii) a T cell epitope capable of binding to HLAT of the subject.

### Personalised Methods of Treatment

According to the present disclosure, the ability of HLAT of a subject to recognise TAAs is predictive of the subject's risk of developing cancer. It follows that a subject's risk of developing cancer may be reduced by stimulating the subject's immune responses using peptides that correspond to the epitopes of TAAs that are recognised by HLAT of the subject.

Accordingly in some cases the disclosure relates to a method of prophylactic treatment of cancer, wherein the method comprises administering to the subject one or more peptides, or one of more polynucleic acids or vectors that encode one or more peptides, that comprise an amino acid sequence that is (i) a fragment of a TAA; and (ii) a T cell epitope capable of binding to HLAT of the subject (i.e. a PEPI3+). In some cases the subject has been determined to be at elevated risk of developing a cancer using a method described herein.

One or more suitable TAA(s) and suitable epitopes in the TAA that bind to HLAT of the subject may be selected as described herein. In some cases the method may comprise the step of identifying and/or selecting suitable TAAs, epitopes and/or peptides. Typically one or more of each TAA will be a TAA that is frequently expressed in cancer cells.

In some cases the subject is determined to be at elevated risk of developing a cancer in which cancer cells express a specific TAA. This may be the case if the TAA comprises few epitopes that are PEPI3+ for the specific subject, or the epitopes of the TAA are recognised by few HLAT of the subject. The treatment for the subject may comprise administration of a peptide comprising an amino acid sequence that (i) is a fragment of that TAA and (ii) comprises a T cell epitope capable of binding to one or more HLAT of the subject.

In other cases the subject is determined to be at elevated risk of developing one or more particular types of cancer, for example any of the types of cancer disclosed herein. The treatment for the subject may comprise administration of a peptide comprising an amino acid sequence that (i) is a fragment a TAA that is associated with expression in that cancer type and (ii) comprises a T cell epitope capable of binding to one or more HLAT of the subject.

In some cases the TAA is one that is recognised by few HLAT of the subject. Such treatment will enhance the T cell responses against the TAA. In other cases the TAA may be one that is recognised by multiple HLAT. The subject will generally already be capable of mounting a broad T cell response against such a TAA. This may in particular help to kill cancer cells that frequently co-express the target TAA with other TAAs that might be less well recognised by the HLAT of the subject.

The peptides may be engineered or non-naturally occurring. The fragment and/or the peptide may be up to 50, 45, 40, 35, 30, 25, 20, 15, 14, 13, 12, 11, 10 or 9 amino acids in length. Typically the peptide may be 15 or 20 to 30 or 35 amino acids in length. In some cases the amino acid sequence that corresponds to a fragment of a TAA is flanked at the N and/or C terminus by additional amino acids that are not part of the consecutive sequence of the TAA. In some cases the sequence is flanked by up to 41 or 35 or 30 or 25 or 20 or 15 or 10, or 9 or 8 or 7 or 6 or 5 or 4 or 3 or 2 or 1 additional amino acid at the N and/or C terminus. In other cases each peptide may either consist of a fragment of a TAA, or consist of two or more such fragments arranged end to end (arranged sequentially in the peptide end to end) or overlapping in a single peptide.

In some cases the method of treatment comprises administering to the subject one or more peptides, or one or more nucleic acids or vectors that encode one or more peptides, that comprise at least 2, or 3, or 4, or 5, or 6, or 7, or 8, or 9, or 10, or 11, or 12, or 13, or 14, or 15, or 20, or 25, or 30, or 35, or 40, or 45, or 50 or more different T cell epitopes (PEPIs) that are each (i) comprised in a fragment of a TAA and (ii) capable of binding to HLAT of the subject. In some cases two or more of the PEPIs is comprised in fragments of at least 2, or 3, or 4, or 5, or 6, or 7, or 8, or 9, or 10, or 11, or 12 or more different TAAs. In some cases one or more or each of the TAAs is a TSA and/or CTA.

In some cases one or more of the peptides fragments comprises an amino acid sequence that is a T cell epitope capable of binding to at least three, or at least four HLA class II alleles of the subject. Such a treatment may elicit both a CD8+ T cell response and a CD4+ T cell response in the subject receiving the treatment.

In some cases the method of treatment comprises administering to the subject any one or more of the peptides, or one or more nucleic acids or vectors encoding one of more of the peptides, or administering any of the pharmaceutical compositions as described in any one of PCT/EP2018/055231, PCT/EP2018/055232, PCT/EP2018/055230, EP 3370065 and EP 3369431. In some specific cases the treatment is for the prevention of breast cancer, ovarian cancer or colorectal cancer and comprises administration of a compositions described in PCT/EP2018/055230 and/or EP 3369431.

As used herein, the term “polypeptide” refers to a full-length protein, a portion of a protein, or a peptide characterized as a string of amino acids. The term “peptide” refers to a short polypeptide. The terms “fragment” or “fragment of a polypeptide” as used herein refer to a string of amino acids or an amino acid sequence typically of reduced length relative to the or a reference polypeptide and comprising, over the common portion, an amino acid sequence identical to the reference polypeptide. Such a fragment according to the disclosure may be, where appropriate, included in a larger polypeptide of which it is a constituent. In some cases the fragment may comprise the full length of the polypeptide, for example where the whole polypeptide, such as a 9 amino acid peptide, is a single T cell epitope. In some cases a peptide or a fragment of a polypeptide may be between 7, or 8, or 9, or 10, or 11, or 12, or 13, or 14, or 15 and 10, or 11, or 12, or 13, or 14, or 15, or 20, or 25, or 30, or 35, or 40, or 45, or 50 amino acids in length.

### Pharmaceutical Compositions and Modes of Administration

In some cases the disclosure relates to a method of treatment comprising administering to a subject one or more peptides as described herein. The one or more peptides may be administered to the subject together or sequentially. For example the treatment may comprise administration of a number of peptides over a period of, for example, up to a year. In some cases a treatment cycle may also be repeated, to boost the immune response.

In addition to the one or more peptides, a pharmaceutical composition for administration to the subject may comprise a pharmaceutically acceptable excipient, carrier, diluent, buffer, stabiliser, preservative, adjuvant or other materials well known to those skilled in the art. Such materials are preferably non-toxic and preferably do not interfere with the pharmaceutical activity of the active ingredient(s). The pharmaceutical carrier or diluent may be, for example, water containing solutions. The precise nature of the carrier or other material may depend on the route of administration, e.g. oral, intravenous, cutaneous or subcutaneous, nasal, intramuscular, intradermal, and intraperitoneal routes.

In order to increase the immunogenicity of the composition, the pharmacological compositions may comprise one or more adjuvants and/or cytokines.

Suitable adjuvants include an aluminum salt such as aluminum hydroxide or aluminum phosphate, but may also be a salt of calcium, iron or zinc, or may be an insoluble suspension of acylated tyrosine, or acylated sugars, or may be cationically or anionically derivatised saccharides, polyphosphazenes, biodegradable microspheres, monophosphoryl lipid A (MPL), lipid A derivatives (e.g. of reduced toxicity), 3-O-deacylated MPL [3D-MPL], quil A, Saponin, QS21, Freund's Incomplete Adjuvant (Difco Laboratories, Detroit, Mich.), Merck Adjuvant 65 (Merck and Company, Inc., Rahway, N.J.), AS-2 (Smith-Kline Beecham, Philadelphia, Pa.), CpG oligonucleotides, bioadhesives and mucoadhesives, microparticles, liposomes, polyoxyethylene ether formulations, polyoxyethylene ester formulations, muramyl peptides or imidazoquinolone compounds (e.g. imiquamod and its homologues). Human immunomodulators suitable for use as adjuvants in the disclosure include cytokines such as interleukins (e.g. IL-1, IL-2, IL-4, IL-5, IL-6, IL-7, IL-12, etc), macrophage colony stimulating factor (M-CSF), tumour necrosis factor (TNF), granulocyte, macrophage colony stimulating factor (GM-CSF) may also be used as adjuvants.

In some embodiments, the compositions comprise an adjuvant selected from the group consisting of Montanide ISA-51 (Seppic, Inc., Fairfield, N.J., United States of America), QS-21 (Aquila Biopharmaceuticals, Inc., Lexington, Mass., United States of America), GM-CSF, cyclophosamide, bacillus Calmette-Guerin (BCG), Corynbacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, peptides, oil emulsions, dinitrophenol, diphtheria toxin (DT).

Examples of suitable compositions of polypeptide fragments and methods of administration are provided in Esseku and Adeyeye (2011) and Van den Mooter G. (2006). Vaccine and immunotherapy composition preparation is generally described in Vaccine Design (“The subunit and adjuvant approach” (eds Powell M. F. & Newman M. J. (1995) Plenum Press New York). Encapsulation within liposomes, which is also envisaged, is described by Fullerton, U.S. Pat. No. 4,235,877.

The method of treatment may comprise administering to the subject a pharmaceutical composition comprising one or more peptides as described herein as active ingredients. The term “active ingredient” as used herein refers to a peptide that is intended to induce an immune response in a subject to which the pharmaceutical composition may be administered. The active ingredient peptide may in some cases be a peptide product of a vaccine or immunotherapy composition that is produced in vivo after administration to a subject. For a DNA or RNA immunotherapy composition, the peptide may be produced in vivo by the cells of a subject to whom the composition is administered. For a cell-based composition, the polypeptide may be processed and/or presented by cells of the composition, for example autologous dendritic cells or antigen presenting cells pulsed with the polypeptide or comprising an expression construct encoding the polypeptide.

In some embodiments, the compositions disclosed herein may be prepared as a nucleic acid vaccine. In some embodiments, the nucleic acid vaccine is a DNA vaccine. In some embodiments, DNA vaccines, or gene vaccines, comprise a plasmid with a promoter and appropriate transcription and translation control elements and a nucleic acid sequence encoding one or more polypeptides of the disclosure. In some embodiments, the plasmids also include sequences to enhance, for example, expression levels, intracellular targeting, or proteasomal processing. In some embodiments, DNA vaccines comprise a viral vector containing a nucleic acid sequence encoding one or more polypeptides of the disclosure. In additional aspects, the compositions disclosed herein comprise one or more nucleic acids encoding peptides determined to have immunoreactivity with a biological sample. For example, in some embodiments, the compositions comprise one or more nucleotide sequences encoding 1, 2, 3, 4, 5, 6, 7, 8, 9, 10, 11, 12, 13, 14, 15, 16, 17, 18, 19, 20, or more peptides comprising a fragment that is a T cell epitope capable of binding to at least three HLA class I molecules of a patient. In some embodiments the DNA or gene vaccine also encodes immunomodulatory molecules to manipulate the resulting immune responses, such as enhancing the potency of the vaccine, stimulating the immune system or reducing immunosuppression. Strategies for enhancing the immunogenicity of DNA or gene vaccines include encoding of xenogeneic versions of antigens, fusion of antigens to molecules that activate T cells or trigger associative recognition, priming with DNA vectors followed by boosting with viral vector, and utilization of immunomodulatory molecules. In some embodiments, the DNA vaccine is introduced by a needle, a gene gun, an aerosol injector, with patches, via microneedles, by abrasion, among other forms. In some forms the DNA vaccine is incorporated into liposomes or other forms of nanobodies. In some embodiments, the DNA vaccine includes a delivery system selected from the group consisting of a transfection agent; protamine; a protamine liposome; a polysaccharide particle; a cationic nanoemulsion; a cationic polymer; a cationic polymer liposome; a cationic nanoparticle; a cationic lipid and cholesterol nanoparticle; a cationic lipid, cholesterol, and PEG nanoparticle; a dendrimer nanoparticle. In some embodiments, the DNA vaccines is administered by inhalation or ingestion. In some embodiments, the DNA vaccine is introduced into the blood, the thymus, the pancreas, the skin, the muscle, a tumor, or other sites.

In some embodiments, the compositions disclosed herein are prepared as an RNA vaccine. In some embodiments, the RNA is non-replicating mRNA or virally derived, self-amplifying RNA. In some embodiments, the non-replicating mRNA encodes the peptides disclosed herein and contains 5′ and 3′ untranslated regions (UTRs). In some embodiments, the virally derived, self-amplifying RNA encodes not only the peptides disclosed herein but also the viral replication machinery that enables intracellular RNA amplification and abundant protein expression. In some embodiments, the RNA is directly introduced into the individual. In some embodiments, the RNA is chemically synthesized or transcribed in vitro. In some embodiments, the mRNA is produced from a linear DNA template using a T7, a T3, or an Sp6 phage RNA polymerase, and the resulting product contains an open reading frame that encodes the peptides disclosed herein, flanking UTRs, a 5′ cap, and a poly(A) tail. In some embodiments, various versions of 5′ caps are added during or after the transcription reaction using a vaccinia virus capping enzyme or by incorporating synthetic cap or anti-reverse cap analogues. In some embodiments, an optimal length of the poly(A) tail is added to mRNA either directly from the encoding DNA template or by using poly(A) polymerase. The RNA encodes one or more peptides comprising a fragment that is a T cell epitope capable of binding to at least three HLA class I molecules of a patient. In some embodiments, the RNA includes signals to enhance stability and translation. In some embodiments, the RNA also includes unnatural nucleotides to increase the half-life or modified nucleosides to change the immunostimulatory profile. In some embodiments, the RNAs is introduced by a needle, a gene gun, an aerosol injector, with patches, via microneedles, by abrasion, among other forms. In some forms the RNA vaccine is incorporated into liposomes or other forms of nanobodies that facilitate cellular uptake of RNA and protect it from degradation. In some embodiments, the RNA vaccine includes a delivery system selected from the group consisting of a transfection agent; protamine; a protamine liposome; a polysaccharide particle; a cationic nanoemulsion; a cationic polymer; a cationic polymer liposome; a cationic nanoparticle; a cationic lipid and cholesterol nanoparticle; a cationic lipid, cholesterol, and PEG nanoparticle; a dendrimer nanoparticle; and/or naked mRNA; naked mRNA with in vivo electroporation; protamine-complexed mRNA; mRNA associated with a positively charged oil-in-water cationic nanoemulsion; mRNA associated with a chemically modified dendrimer and complexed with polyethylene glycol (PEG)-lipid; protamine-complexed mRNA in a PEG-lipid nanoparticle; mRNA associated with a cationic polymer such as polyethylenimine (PEI); mRNA associated with a cationic polymer such as PEI and a lipid component; mRNA associated with a polysaccharide (for example, chitosan) particle or gel; mRNA in a cationic lipid nanoparticle (for example, 1,2-dioleoyloxy-3-trimethylammoniumpropane (DOTAP) or dioleoylphosphatidylethanolamine (DOPE) lipids); mRNA complexed with cationic lipids and cholesterol; or mRNA complexed with cationic lipids, cholesterol and PEG-lipid. In some embodiments, the RNA vaccine is administered by inhalation or ingestion. In some embodiments, the RNA is introduced into the blood, the thymus, the pancreas, the skin, the muscle, a tumor, or other sites, and/or by an intradermal, intramuscular, subcutaneous, intranasal, intranodal, intravenous, intrasplenic, intratumoral or other delivery route.

Polynucleotide or oligonucleotide components may be naked nucleotide sequences, or be in combination with cationic lipids, polymers or targeting systems. They may be delivered by any available technique. For example, the polynucleotide or oligonucleotide is introduced by needle injection, preferably intradermally, subcutaneously or intramuscularly. Alternatively, the polynucleotide or oligonucleotide is delivered directly across the skin using a delivery device such as particle-mediated gene delivery. The polynucleotide or oligonucleotide may be administered topically to the skin, or to mucosal surfaces for example by intranasal, oral, or intrarectal administration.

Uptake of polynucleotide or oligonucleotide constructs may be enhanced by several known transfection techniques, for example those including the use of transfection agents. Examples of these agents include cationic agents, for example, calcium phosphate and DEAE-Dextran and lipofectants, for example, lipofectam and transfectam. The dosage of the polynucleotide or oligonucleotide to be administered can be altered.

Administration is typically in a “prophylactically effective amount” or a “therapeutically effective amount” (as the case may be, although prophylaxis may be considered therapy), this being sufficient to result in a clinical response or to show clinical benefit to the individual, e.g. an effective amount to prevent or delay onset of the disease or condition, to ameliorate one or more symptoms, to induce or prolong remission, or to delay relapse or recurrence. In some cases the methods of treatment according to the disclosure may be performed for the prophylaxis of cancer recurrence or metastasis in persons with a cured primary cancer disease.

The dose may be determined according to various parameters, especially according to the substance used; the age, weight and condition of the individual to be treated; the route of administration; and the required regimen. The amount of antigen in each dose is selected as an amount which induces an immune response. A physician will be able to determine the required route of administration and dosage for any particular individual. The dose may be provided as a single dose or may be provided as multiple doses, for example taken at regular intervals, for example 2, 3 or 4 doses administered hourly. Typically peptides, polynucleotides or oligonucleotides are typically administered in the range of 1 pg to 1 mg, more typically 1 pg to 10 μg for particle mediated delivery and 1 μg to 1 mg, more typically 1-100 μg, more typically 5-50 μg for other routes. Generally, it is expected that each dose will comprise 0.01-3 mg of antigen. An optimal amount for a particular vaccine can be ascertained by studies involving observation of immune responses in subjects.

Examples of the techniques and protocols mentioned above can be found in Remington's Pharmaceutical Sciences, 20th Edition, 2000, pub. Lippincott, Williams & Wilkins.

Routes of administration include but are not limited to intranasal, oral, subcutaneous, intradermal, and intramuscular. Typically administration is subcutaneous. Subcutaneous administration may for example be by injection into the abdomen, lateral and anterior aspects of upper arm or thigh, scapular area of back, or upper ventrodorsal gluteal area.

The skilled artisan will recognize that the composition may also be administered in one, or more doses, as well as, by other routes of administration. For example, such other routes include, intracutaneously, intravenously, intravascularly, intraarterially, intraperitnoeally, intrathecally, intratracheally, intracardially, intralobally, intramedullarly, intrapulmonarily, and intravaginally. Depending on the desired duration of the treatment, the compositions according to the disclosure may be administered once or several times, also intermittently, for instance on a monthly basis for several months or years and in different dosages.

The methods of treatment according to the disclosure may be performed alone or in combination with other pharmacological compositions or treatments, for example behavioural or lifestyle changes, chemotherapy, immunotherapy and/or vaccine. The other therapeutic compositions or treatments may for example be one or more of those discussed herein, and may be administered either simultaneously or sequentially with (before or after) the composition or treatment of the disclosure.

In some cases the treatment may be administered in combination with surgery, chemotherapy, cytotoxic or non-cytotoxic chemotherapy, radiation therapy, targeted therapy, hormone therapy, or the administration of targeted small-molecule drugs or antibodies, e.g. monoclonal antibodies or co-stimulatory antibodies. It has been demonstrated that chemotherapy sensitizes tumors to be killed by tumor specific cytotoxic T cells induced by vaccination (Ramakrishnan et al. J Clin Invest. 2010; 120(4):1111-1124). Examples of chemotherapy agents include alkylating agents including nitrogen mustards such as mechlorethamine (HN2), cyclophosphamide, ifosfamide, melphalan (L-sarcolysin) and chlorambucil; anthracyclines; epothilones; nitrosoureas such as carmustine (BCNU), lomustine (CCNU), semustine (methyl-CCNU) and streptozocin (streptozotocin); triazenes such as decarbazine (DTIC; dimethyltriazenoimidazole-carboxamide; ethylenimines/methylmelamines such as hexamethylmelamine, thiotepa; alkyl sulfonates such as busulfan; Antimetabolites including folic acid analogues such as methotrexate (amethopterin); alkylating agents, antimetabolites, pyrimidine analogs such as fluorouracil (5-fluorouracil; 5-FU), floxuridine (fluorodeoxyuridine; FUdR) and cytarabine (cytosine arabinoside); purine analogues and related inhibitors such as mercaptopurine (6-mercaptopurine; 6-MP), thioguanine (6-thioguanine; TG) and pentostatin (2′-deoxycoformycin); epipodophylotoxins; enzymes such as L-asparaginase; biological response modifiers such as IFNα, IL-2, G-CSF and GM-CSF; platinum coordination complexes such as cisplatin (cis-DDP), oxaliplatin and carboplatin; anthracenediones such as mitoxantrone and anthracycline; substituted urea such as hydroxyurea; methylhydrazine derivatives including procarbazine (N-methylhydrazine, MIH) and procarbazine; adrenocortical suppressants such as mitotane (o,p′-DDD) and aminoglutethimide; taxol and analogues/derivatives; hormones/hormonal therapy and agonists/antagonists including adrenocorticosteroid antagonists such as prednisone and equivalents, dexamethasone and aminoglutethimide, progestin such as hydroxyprogesterone caproate, medroxyprogesterone acetate and megestrol acetate, estrogen such as diethylstilbestrol and ethinyl estradiol equivalents, antiestrogen such as tamoxifen, androgens including testosterone propionate and fluoxymesterone/equivalents, antiandrogens such as flutamide, gonadotropin-releasing hormone analogs and leuprolide and non-steroidal antiandrogens such as flutamide; natural products including vinca alkaloids such as vinblastine (VLB) and vincristine, epipodophyllotoxins such as etoposide and teniposide, antibiotics such as dactinomycin (actinomycin D), daunorubicin (daunomycin; rubidomycin), doxorubicin, bleomycin, plicamycin (mithramycin) and mitomycin (mitomycin C), enzymes such as L-asparaginase, and biological response modifiers such as interferon alphenomes.

### Systems

The disclosure provides a system. The system may comprise a storage module configured to store data comprising the HLA class I genotype of a subject and the amino acid sequences of TAAs. The system may comprise a computation module configured to quantify the HLAT of the subject that are capable of binding to T cell epitopes in the amino acid sequence of the TAAs, wherein each HLA of a HLAT is capable of binding to the same T cell epitope. The system may comprise a module for receiving at least one sample from at least one subject. The system may comprise a HLA genotyping module for determining the class I and/or class II HLA genotype of a subject. The storage module may be configured to store the data output from the genotyping module. The HLA genotyping module may receive a biological sample obtained from the subject and determines the subject's class I and/or class II HLA genotype. The sample typically contains subject DNA. The sample may be, for example, a blood, serum, plasma, saliva, urine, expiration, cell or tissue sample. The system may further comprise an output module configured to display an indication of the risk that the subject will develop a cancer and/or a recommended treatment for the subject as described herein.

### Further Embodiments of the Disclosure

1. A method for treating a human subject at risk of developing a cancer, the method comprising
   a. quantifying the HLA triplets (HLAT) of the subject that are capable of binding to T cell epitopes in the amino acid sequence of tumor associated antigens (TAAs), wherein each HLA of a HLAT is capable of binding to the same T cell epitope;
   b. determining the risk that the subject will develop a cancer, wherein, with respect to a TAA, a lower number of HLATs capable of binding to T cell epitopes of the TAA corresponds to a higher risk that the subject will develop cancer; and
   c. administering to the subject a peptide, or a polynucleic acid or vector that encodes a peptide, that comprises an amino acid sequence that
      i. is a fragment of a TAA; and
      ii. comprises a T cell epitope capable of binding to an HLAT of the subject.
2. The method of item 1, wherein the TAA fragment is flanked at the N and/or C terminus by additional amino acids that are not part of the sequence of the TAA.
3. The method according to any one of items 1 to 2, wherein the cancer is selected from melanoma, lung cancer, renal cell cancer, colorectal cancer, bladder cancer, glioma, head and neck cancer, ovarian cancer, non-melanoma skin cancer, prostate cancer, kidney cancer, stomach cancer, liver cancer, cervix uteri cancer, oesophagus cancer, non-Hodgkin lymphoma, leukemia, pancreatic cancer, corpus uteri cancer, lip cancer, oral cavity cancer, thyroid cancer, brain cancer, nervous system cancer, gallbladder cancer, larynx cancer, pharynx cancer, myeloma, nasopharynx cancer, Hodgkin lymphoma, testis cancer, breast cancer, and Kaposi sarcoma.
4. The method according to item 1, wherein the TAA are selected from any one of those listed in Table 2 or Table 11.
5. A method for treating cancer in an individual in need thereof with a cancer treatment, comprising:
   determining whether the individual is at a higher risk of developing cancer by:
      performing a quantification assay on a biological sample from the individual to determine the HLA triplets (HLAT) of the individual that are capable of binding to T cell epitopes in the amino acid sequence of tumor associated antigens (TAAs), wherein each HLA of a HLAT is capable of binding to the same T cell epitope; and
      if the individual has a lower number of HLATs capable of binding to T cell epitopes of the TAAs than a threshold derived from a cohort of control individuals, then administering to the individual the cancer treatment.
6. The method of item 5, further comprising obtaining the biological sample from the individual.
7. The method of item 5, wherein the cancer is selected from melanoma, lung cancer, renal cell cancer, colorectal cancer, bladder cancer, glioma, head and neck cancer, ovarian cancer, non-melanoma skin cancer, prostate cancer, kidney cancer, stomach cancer, liver cancer, cervix uteri cancer, oesophagus cancer, non-Hodgkin lymphoma, leukemia, pancreatic cancer, corpus uteri cancer, lip cancer, oral cavity cancer, thyroid cancer, brain cancer, nervous system cancer, gallbladder cancer, larynx cancer, pharynx cancer, myeloma, nasopharynx cancer, Hodgkin lymphoma, testis cancer, breast cancer, and Kaposi sarcoma.
8. The method of item 5, wherein the cancer treatment comprises administering to the individual a peptide, or a polynucleic acid or vector that encodes a peptide, that comprises an amino acid sequence that
   (i) is a fragment of a TAA; and
   (ii) comprises a T cell epitope capable of binding to an HLAT of the individual.
9. The method of item 8, wherein the TAA fragment is flanked at the N and/or C terminus by additional amino acids that are not part of the sequence of the TAA.
10. The method of item 5, wherein the TAAs are selected from any one of those listed in Table 2 or Table 11.
11. The method of item 5, wherein the biological sample comprises blood, serum, plasma, saliva, urine, expiration, cell, or tissue.
12. A method for treating cancer in an individual in need thereof, comprising: administering a cancer treatment to an individual having a lower number of HLA triplets (HLATs) that are capable of binding to T cell epitopes of the tumor associated antigens (TAA) than a threshold derived from a cohort of control individuals.
13. The method of item 12, wherein the cancer treatment comprises administering to the individual a peptide, or a polynucleic acid or vector that encodes a peptide, that comprises an amino acid sequence that
   (i) is a fragment of a TAA; and
   (ii) comprises a T cell epitope capable of binding to an HLAT of the individual; optionally wherein the TAA fragment is flanked at the N and/or C terminus by additional amino acids that are not part of the sequence of the TAA.
14. The method of item 12, wherein the TAAs are selected from any one of those listed in Table 2 or Table 11.
15. The method of item 12, wherein the cancer is selected from melanoma, lung cancer, renal cell cancer, colorectal cancer, bladder cancer, glioma, head and neck cancer, ovarian cancer, non-melanoma skin cancer, prostate cancer, kidney cancer, stomach cancer, liver cancer, cervix uteri cancer, oesophagus cancer, non-Hodgkin lymphoma, leukemia, pancreatic cancer, corpus uteri cancer, lip cancer, oral cavity cancer, thyroid cancer, brain cancer, nervous system cancer, gallbladder cancer, larynx cancer, pharynx cancer, myeloma, nasopharynx cancer, Hodgkin lymphoma, testis cancer, breast cancer, and Kaposi sarcoma.
16. A system for determining the risk that a human subject will develop a cancer, the system comprising:
   (i) a storage module configured to store data comprising the HLA class I genotype of a subject and the amino acid sequences of TAAs;
   (ii) a computation module configured to quantify the HLAT of the subject that are capable of binding to T cell epitopes in the amino acid sequence of the TAAs, wherein each HLA of a HLAT is capable of binding to the same T cell epitope; and
   (iii) an output module configured to display an indication of the risk that the subject will develop a cancer and/or a recommended treatment for the subject.

### EXAMPLES

### Example 1—HLA-Epitope Binding Prediction Process and Validation

Predicted binding between particular HLA and epitopes (9 mer peptides) was based on the Immune Epitope Database tool for epitope prediction (iedb.org).

The HLA I-epitope binding prediction process was validated by comparison with HLA class I-epitope pairs determined by laboratory experiments. A dataset was compiled of HLA I-epitope pairs reported in peer reviewed publications or public immunological databases.

The rate of agreement with the experimentally determined dataset was determined (Table 3). The binding HLA I-epitope pairs of the dataset were correctly predicted with a 93% probability. Coincidentally the non-binding HLA I-epitope pairs were also correctly predicted with a 93% probability.

TABLE 3
Analytical specificity and sensitivity of
the HLA-epitope binding prediction process.

| | True epitopes | False epitopes |
|---|---|---|
| | (n = 327) | (n = 100) |
| HLA-epitope pairs | (Binder match) | (Non-binder match) |
| HIV | 91% (32) | 82% (14) |
| Viral | 100% (35) | 100% (11) |
| Tumor | 90% (172) | 94% (32) |
| Other (fungi, bacteria, etc.) | 100% (65) | 95% (36) |
| All | 93% (304) | 93% (93) |

The accuracy of the prediction of multiple HLA binding epitopes was also determined (Table 4). Based on the analytical specificity and sensitivity using the 93% probability for both true positive and true negative prediction and 7% (=100%-93%) probability for false positive and false negative prediction, the probability of the existence of a multiple HLA binding epitope in a person can be calculated. The probability of multiple HLA binding to an epitope shows the relationship between the number of HLAs binding an epitope and the expected minimum number of real binding. Per PEPI definition three is the expected minimum number of HLA to bind an epitope (bold).

TABLE 4
Accuracy of multiple HLA binding epitopes predictions.

| Expected | | | | | | | |
|---|---|---|---|---|---|---|---|
| minimum | | | | | | | |
| number of real | Predicted number of HLAs binding to an epitope | | | | | | |
| HLA binding | 0 | 1 | 2 | 3 | 4 | 5 | 6 |
| 1 | 35% | 95% | 100% | 100% | 100% | 100% | 100% |
| 2 | 6% | 29% | 90% | 99% | 100% | 100% | 100% |
| 3 | 1% | 4% | 22% | 84% | 98% | 100% | 100% |
| 4 | 0% | 0% | 2% | 16% | 78% | 96% | 99% |
| 5 | 0% | 0% | 0% | 1% | 10% | 71% | 94% |
| 6 | 0% | 0% | 0% | 0% | 0% | 5% | 65% |

The validated HLA-epitope binding prediction process was used to determine all HLA-epitope binding pairs described in the Examples below.

### Example 2—Epitope Presentation by Multiple HLA Predicts Cytotoxic T Lymphocyte (CTL) Response

This study investigates whether the presentation of one or more epitopes of a polypeptide antigen by one or more HLA class I molecule of an individual is predictive for a CTL response.

The study was carried out by retrospective analysis of six clinical trials, conducted on 71 cancer patients and 9 HIV-infected patients (Table 5). Patients from these studies were treated with an HPV vaccine, three different NY-ESO-1 specific cancer vaccines, one HIV-1 vaccine and a CTLA-4 specific monoclonal antibody (Ipilimumab) that was shown to reactivate CTLs against NY-ESO-1 antigen in melanoma patients. All of these clinical trials measured antigen specific CD8+ CTL responses (immunogenicity) in the study subjects after vaccination. In some cases, correlation between CTL responses and clinical responses were reported.

No patient was excluded from the retrospective study for any reason other than data availability. The 157 patient datasets (Table 5) were randomized with a standard random number generator to create two independent cohorts for training and evaluation studies. In some cases, the cohorts contained multiple datasets from the same patient, resulting in a training cohort of 76 datasets from 48 patients and a test/validation cohort of 81 datasets from 51 patients.

TABLE 5
Summary of patient datasets

| | | | | | | Immunoassay | |
|---|---|---|---|---|---|---|---|
| | | | | | # Data sets | performed in | HLA |
| Clinical | | Target | | # | (#antigen × | the clinical | genotyping |
| trial | Immunotherapy | Antigen | Disease | Patients* | #patient) | trials** | method |
| 1 | VGX-3100 | HPV16-E6 | Cervical | 17/18 | 5 × 17 | IFN-γ | High |
| | | HPV16-E7 | cancer | | | ELISPOT | Resolution |
| | | HPV18-E6 | | | | | SBT |
| | | HPV18-E7 | | | | | |
| | | HPV16/18 | | | | | |
| 2 | HIVIS vaccine | HIV-1 Gag | AIDS | 9/12 | 2 × 9 | IFN-γ | Low-Medium |
| | | HIV-1 RT | | | | ELISPOT | Resolution |
| | | | | | | | SSO |
| 3 | rNY-ESO-1 | NY-ESO-1 | Breast-and | 18/18 | 1 × 18 | In vitro and | High |
| | | | ovarian | | | Ex vivo IFN-γ | Resolution |
| | | | cancers, | | | ELISPOT | SBT |
| | | | melanoma | | | | |
| | | | and sarcoma | | | | |
| 4 | Ipilimumab | NY-ESO-1 | Metastatic | 19/20 | 1 × 19 | ICS after | Low to medium |
| | | | melanoma | | | T-cell | resolution |
| | | | | | | stimulation | typing, SSP of |
| | | | | | | | genomic DNA, |
| | | | | | | | high resolution |
| | | | | | | | sequencing |
| 5 | NY-ESO-1f | NY-ESO-1 | Esophageal-, | 10/10 | 1 × 10 | ICS after | SSO probing |
| | | (91-110) | non-small- | | | T-cell | and SSP of |
| | | | cell lung- | | | stimulation | genomic DNA |
| | | | and gastric | | | | |
| | | | cancer | | | | |
| 6 | NY-ESO-1 | NY-ESO-1 | Esophageal- | 7/9 | 1 × 7 | ICS after | SSO probing |
| | overlapping | (79-173) | and lung | | | T-cell | and SSP of |
| | peptides | | cancer, | | | stimulation | genomic DNA |
| | | | malignant | | | | |
| | | | melanoma | | | | |
| Total | 6 | 7 | | 80 | 157 | N/A | |

The reported CD8+ T cell responses of the training dataset were compared with the HLA class I restriction profile of epitopes (9 mers) of the vaccine antigens. The antigen sequences and the HLA class I genotype of each patient were obtained from publicly available protein sequence databases or peer reviewed publications and the HLA I-epitope binding prediction process was blinded to patients' clinical CD8+ T cell response data where CD8+ T cells are IFN-γ producing CTL specific for vaccine peptides (9 mers). The number of epitopes from each antigen predicted to bind to at least 1 (PEPI1+), or at least 2 (PEPI2+), or at least 3 (PEPI3+), or at least 4 (PEPI4+), or at least 5 (PEPI5+), or all 6 (PEPI6) HLA class I molecules of each patient was determined and the number of HLA bound were used as classifiers for the reported CTL responses. The true positive rate (sensitivity) and true negative rate (specificity) were determined from the training dataset for each classifier (number of HLA bound) separately.

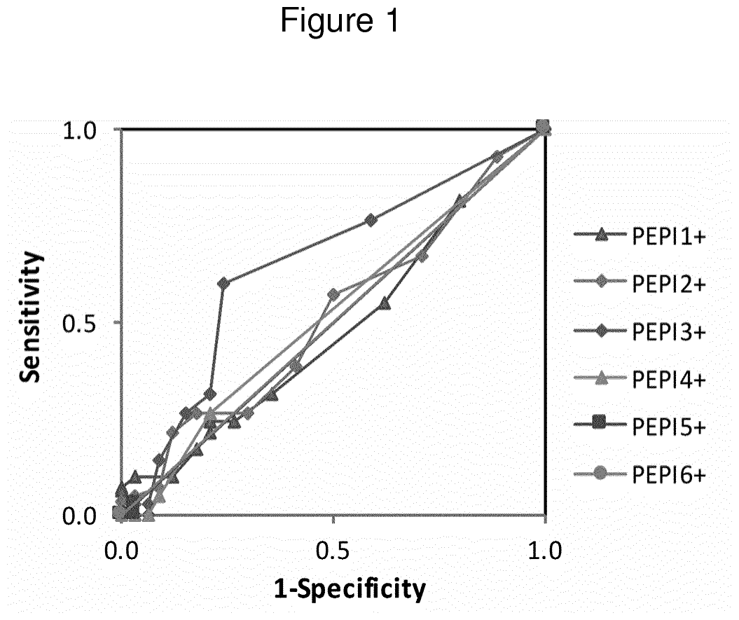

ROC analysis was performed for each classifier. In a ROC curve, the true positive rate (Sensitivity) was plotted in function of the false positive rate (1-Specificity) for different cut-off points (FIG. 1). Each point on the ROC curve represents a sensitivity/specificity pair corresponding to a particular decision threshold (epitope (PEPI) count). The area under the ROC curve (AUC) is a measure of how well the classifier can distinguish between two diagnostic groups (CTL responder or non-responder).

The analysis unexpectedly revealed that predicted epitope presentation by multiple class I HLAs of a subject (PEPI2+, PEPI3+, PEPI4+, PEPI5+, or PEPI6), was in every case a better predictor of the CD8+ T cell response or CTL response than epitope presentation by merely one or more HLA class I (PEPI1+, AUC=0.48, Table 6).

TABLE 6
Determination of diagnostic value of
the PEPI biomarker by ROC analysis

| Classifiers | AUC |
|---|---|
| PEPI1+ | 0.48 |
| PEPI2+ | 0.51 |
| PEPI3+ | 0.65 |
| PEPI4+ | 0.52 |
| PEPI5+ | 0.5 |
| PEPI6+ | 0.5 |

The CTL response of an individual was best predicted by considering the epitopes of an antigen that could be presented by at least 3 HLA class I alleles of an individual (PEPI3+, AUC=0.65, Table 7). The threshold count of PEPI3+(number of antigen-specific epitopes presented by 3 or more HLA of an individual) that best predicted a positive CTL response was 1 (Table 7). In other words, at least one antigen-derived epitope is presented by at least 3 HLA class I of a subject (≥1 PEPI3+), then the antigen can trigger at least one CTL clone, and the subject is a likely CTL responder. Using the ≥1 PEPI3+ threshold to predict likely CTL responders (“≥1 PEPI3+ test”) provided 76% true positive rate (diagnostic sensitivity) (Table 7).

TABLE 7
Determination of the ≥1 PEPI3+ threshold to predict
likely CTL responders in the training dataset.

| | PEPI3+ Count | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Sensitivity: | 0.76 | 0.60 | 0.31 | 0.26 | 0.14 | 0.02 | 0 | 0 | 0 | 0 | 0 | 0 |
| 1- | 0.59 | 0.24 | 0.21 | 0.15 | 0.09 | 0.06 | 0.06 | 0.03 | 0.03 | 0.03 | 0.03 | 0.03 |

### Example 3—Retrospective Validation of the ≥1 PEPI3+ Threshold as Novel Biomarker for PEPI Test

In a retrospective analysis, the test cohort of 81 datasets from 51 patients was used to validate the ≥1 PEPI3+ threshold to predict an antigen-specific CD8+ T cell response or CTL response. For each dataset in the test cohort it was determined whether the ≥1 PEPI3+ threshold was met (at least one antigen-derived epitope presented by at least three class I HLA of the individual). This was compared with the experimentally determined CD8+ T cell responses (CTL responses) reported from the clinical trials (Table 8).

The retrospective validation demonstrated that a PEPI3+ peptide induces CD8+ T cell response (CTL response) in an individual with 84% probability. 84% is the same value that was determined in the analytical validation of the PEPI3+ prediction, epitopes that binds to at least 3 HLAs of an individual (Table 4). These data provide strong evidences that immune responses are induced by PEPIs in individuals.

TABLE 8
Diagnostic performance characteristics of the ≥1 PEPI3+ Test (n = 81).

| Performance characteristic | | Description | Result |
|---|---|---|---|
| Positive | 100%[A/(A + B)] | The likelihood that an individual that meets the ≥1 | 84% |
| predictive | | PEPI3+ threshold has antigen-specific CTL | |
| value (PPV) | | responses after treatment with immunotherapy. | |
| Sensitivity | 100%[A/(A + C)] | The proportion of subjects with antigen-specific | 75% |
| | | CTL responses after treatment with immunotherapy | |
| | | who meet the ≥1 PEPI3+ threshold. | |
| Specificity | 100%[D/(B + D)] | The proportion of subjects without antigen-specific | 55% |
| | | CTL responses after treatment with immunotherapy | |
| | | who do not meet the ≥1 PEPI3+ threshold. | |
| Negative | 100%[D/(C + D)] | The likelihood that an individual who does not meet | 42% |
| predictive | | the ≥1 PEPI3+ threshold does not have antigen- | |
| value (NPV) | | specific CTL responses after treatment with | |
| | | immunotherapy. | |
| Overall | 100%[(A + D)/N] | The percentage of predictions based on the ≥1 | 70% |
| percent | | PEPI3+ threshold that match the experimentally | |
| agreement (OPA) | | determined result, whether positive or negative. | |
| Fisher's exact (p) | | | 0.01 |

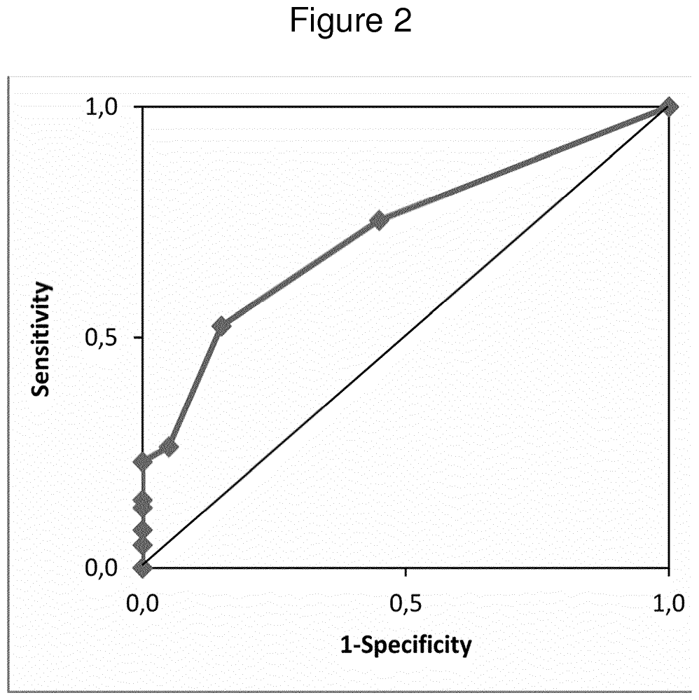

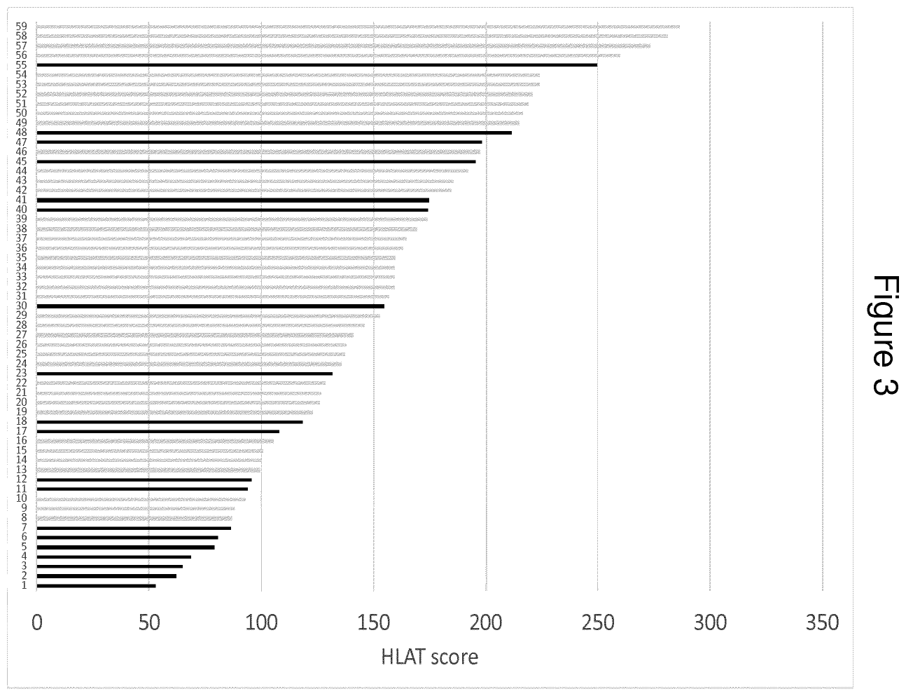

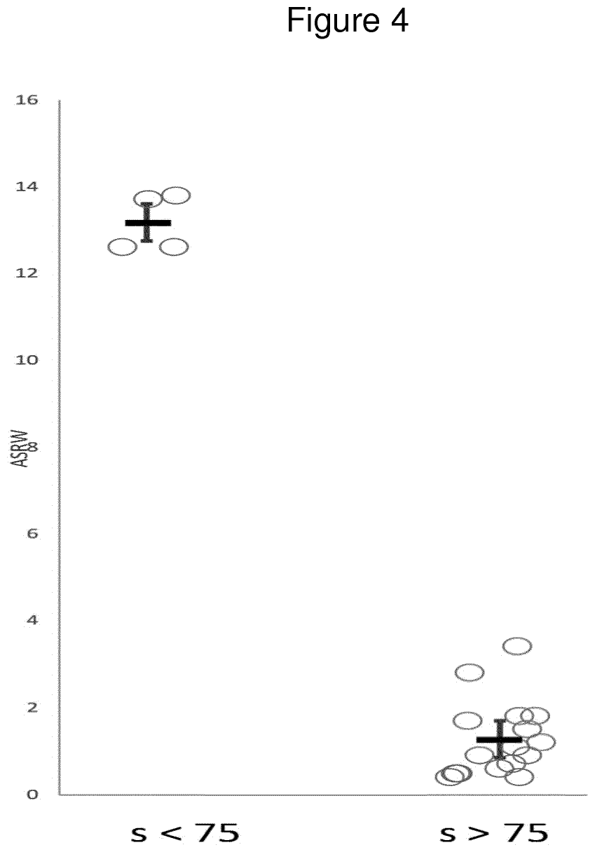

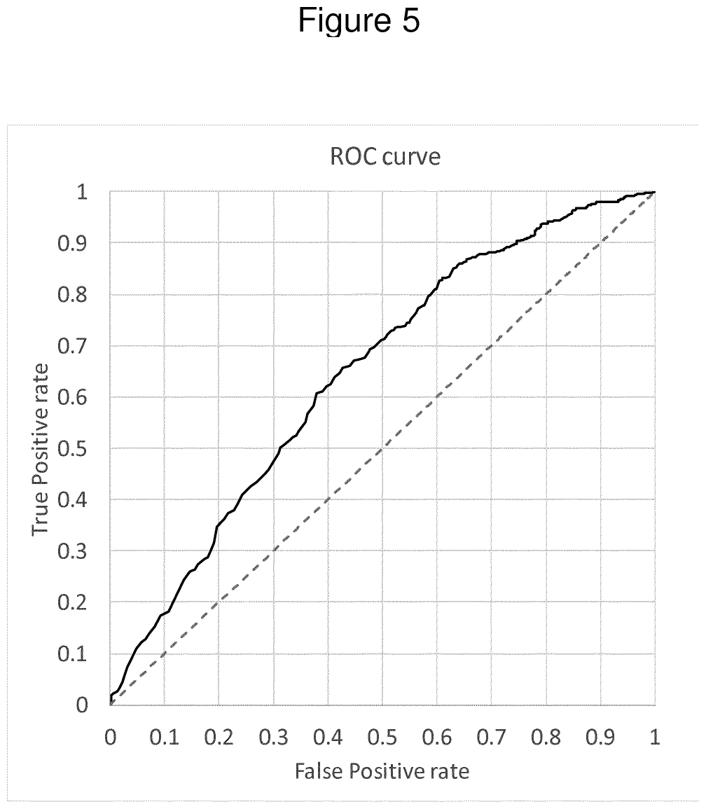

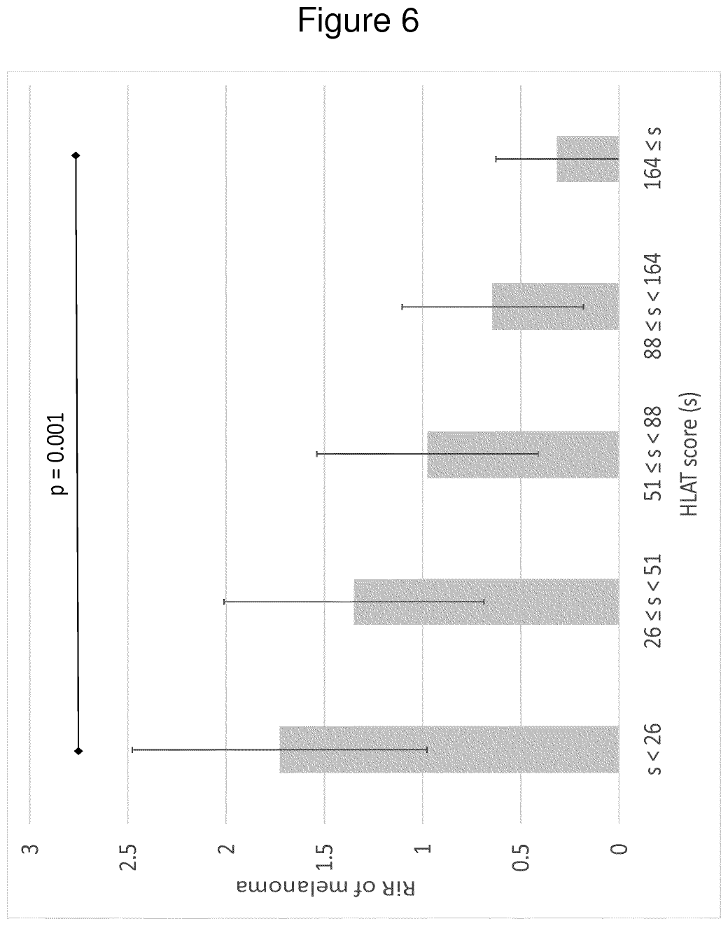

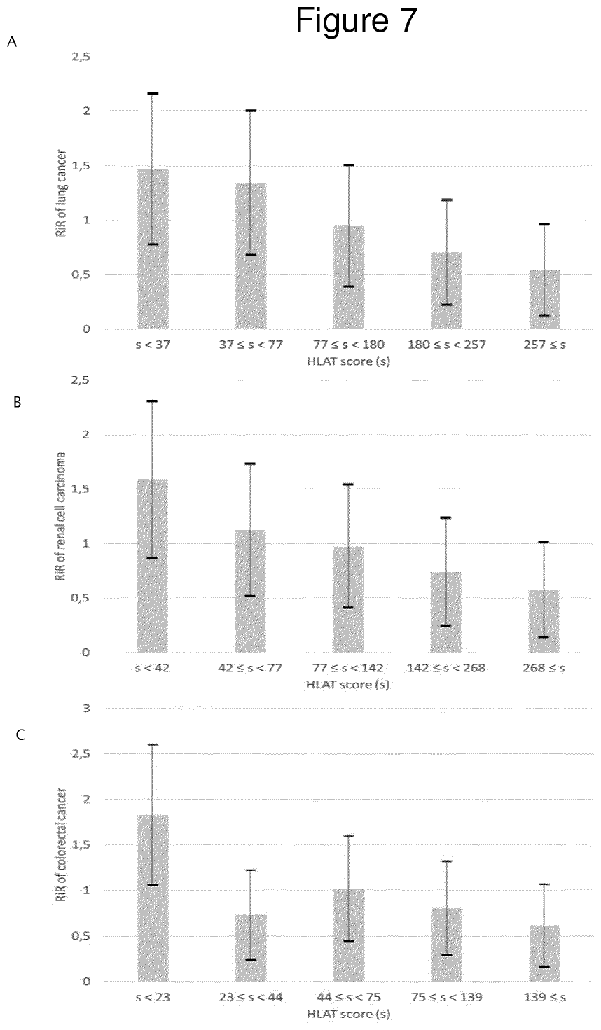

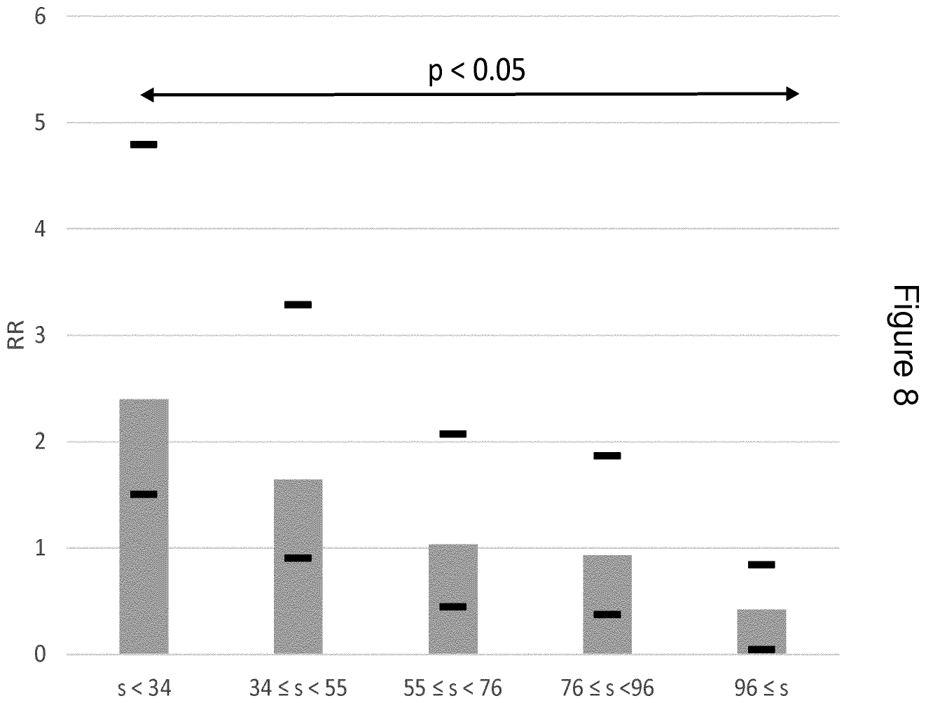

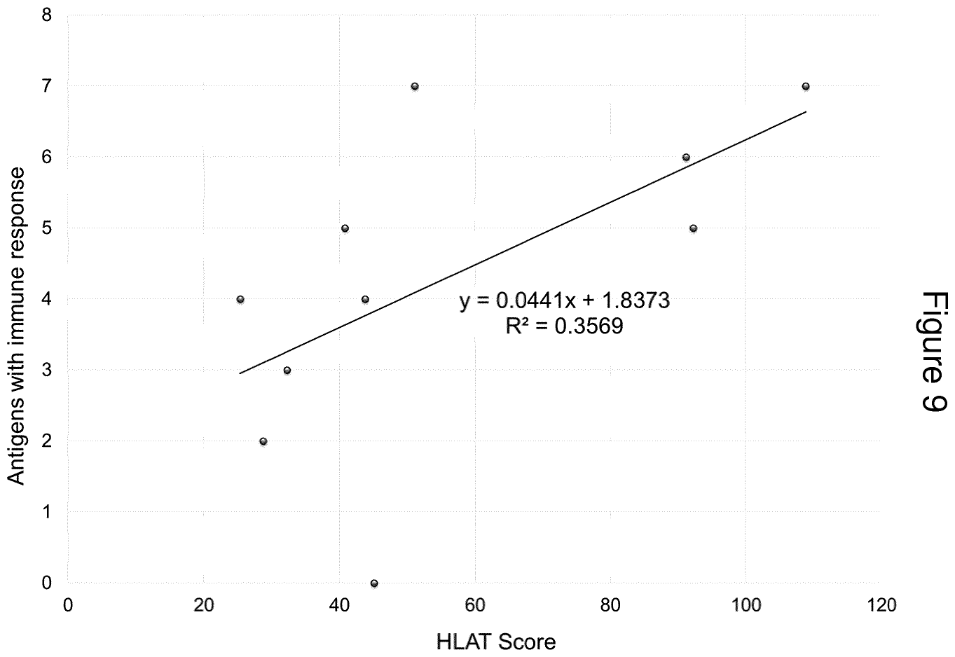

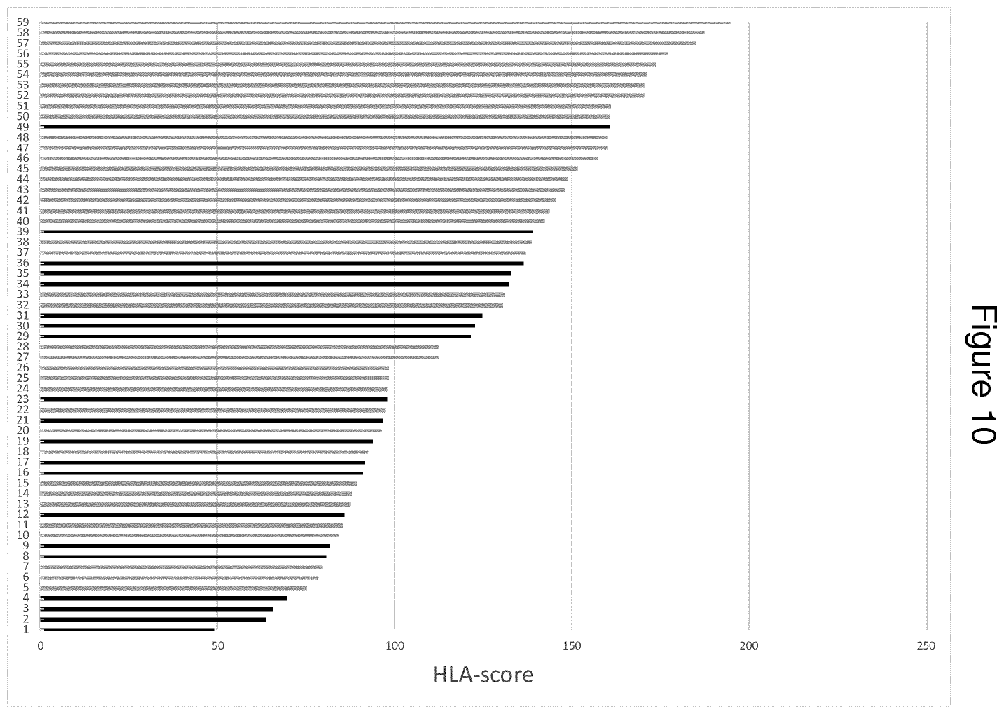

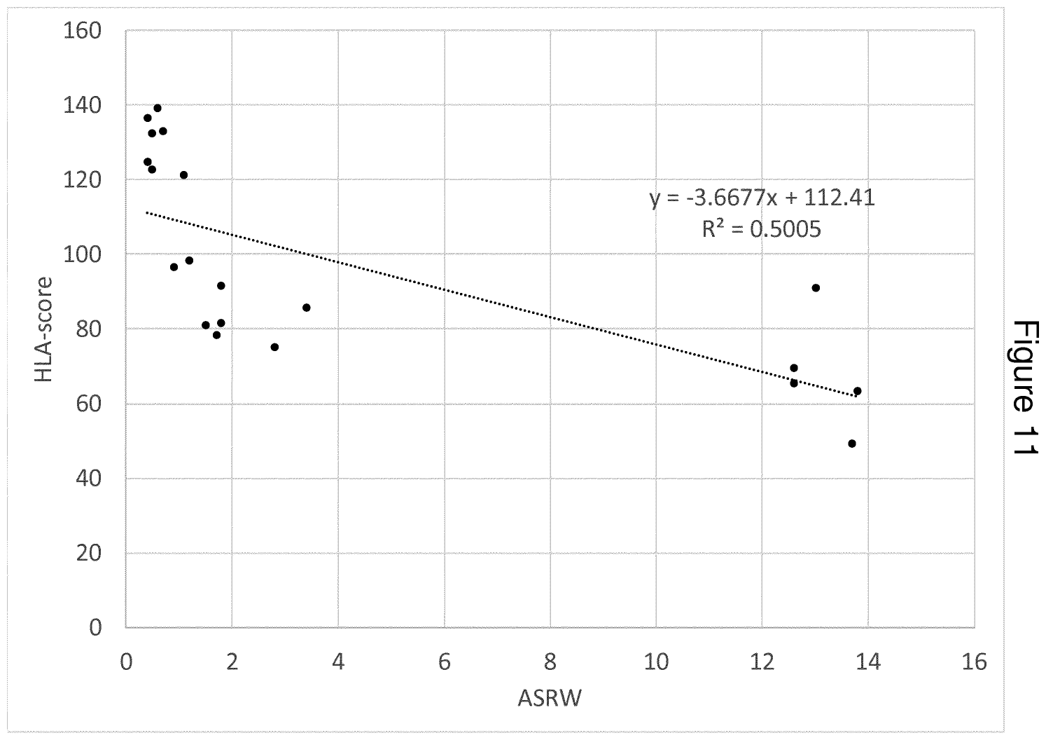

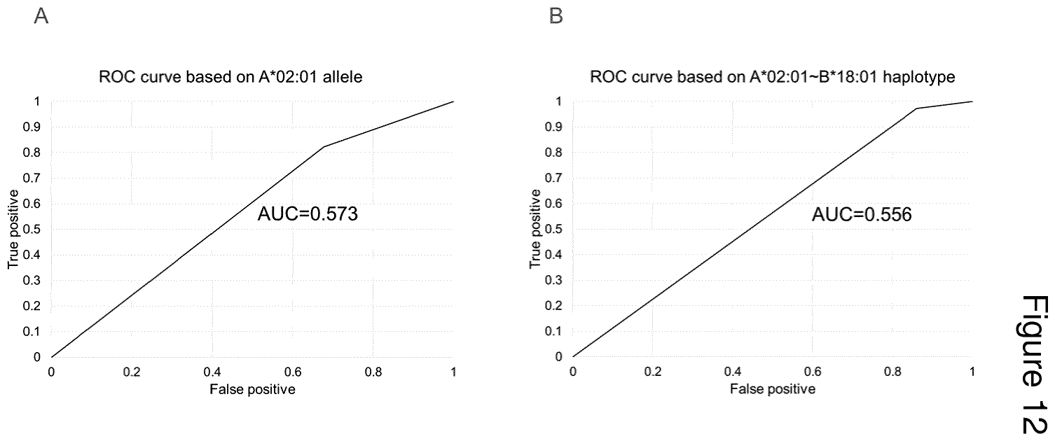

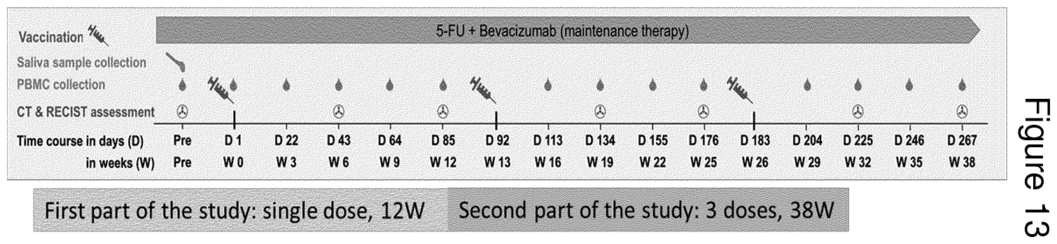

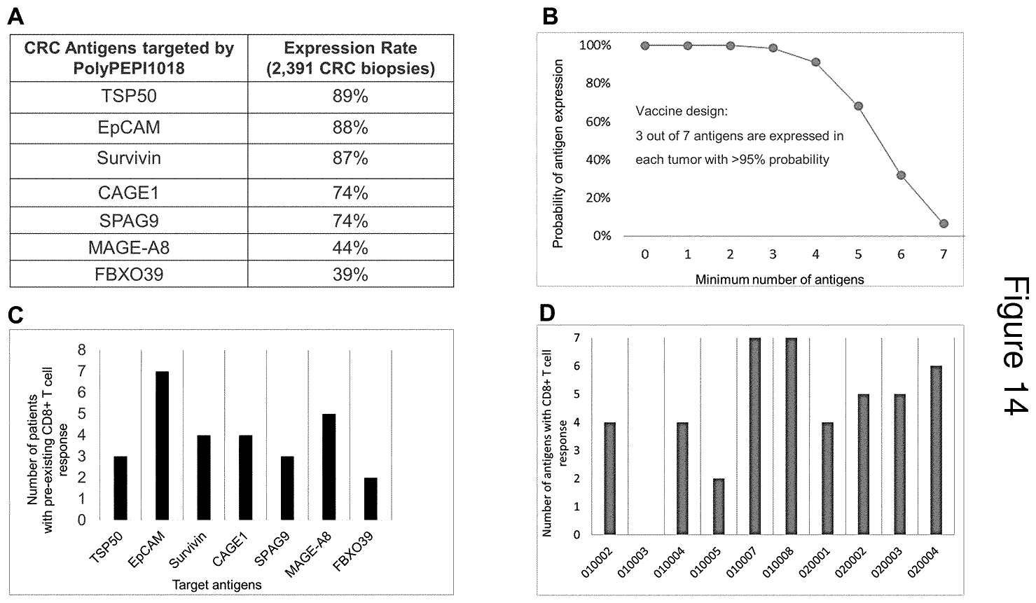

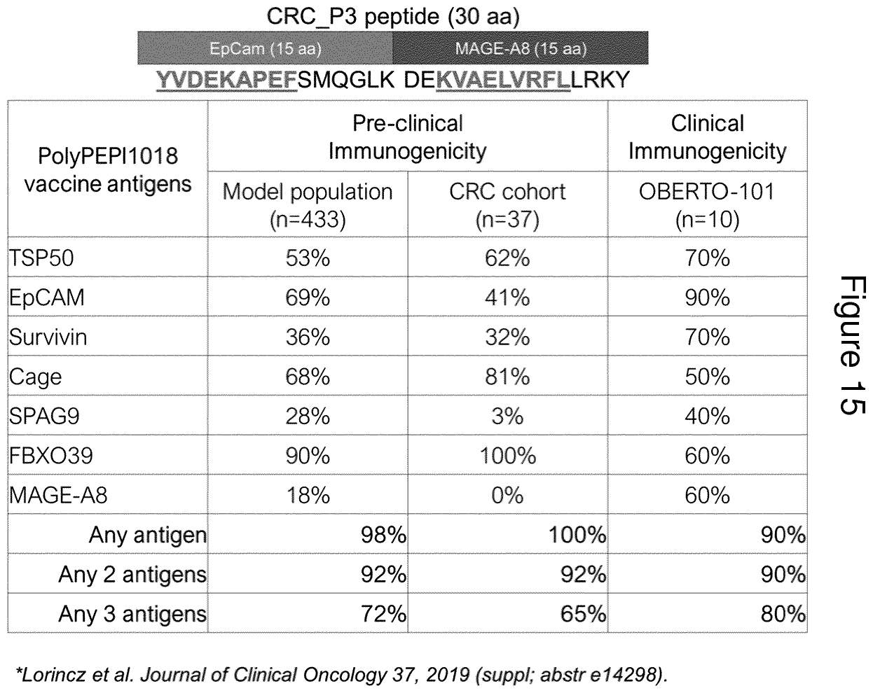

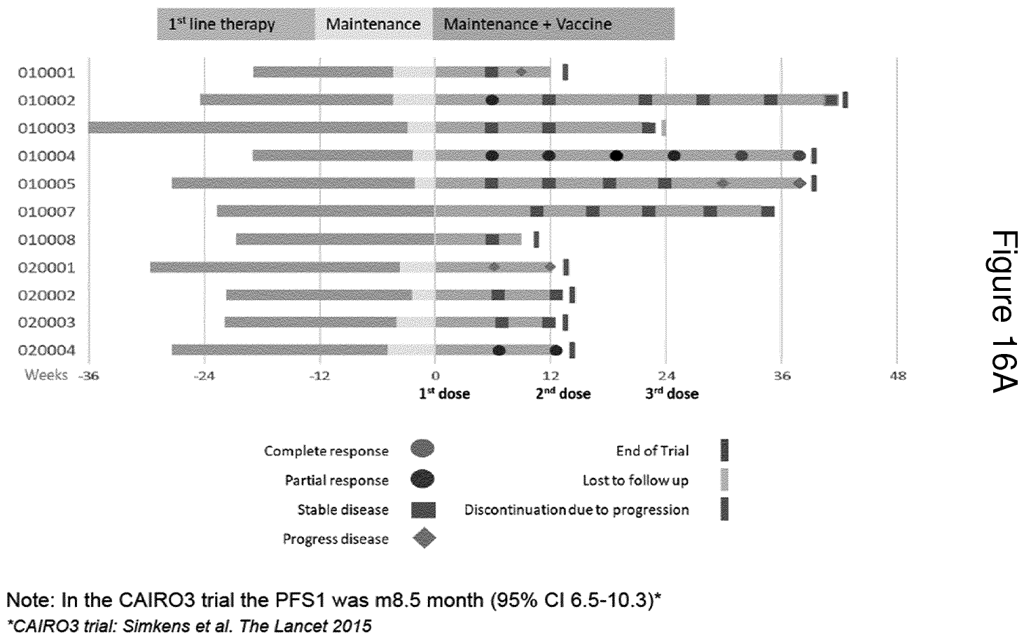

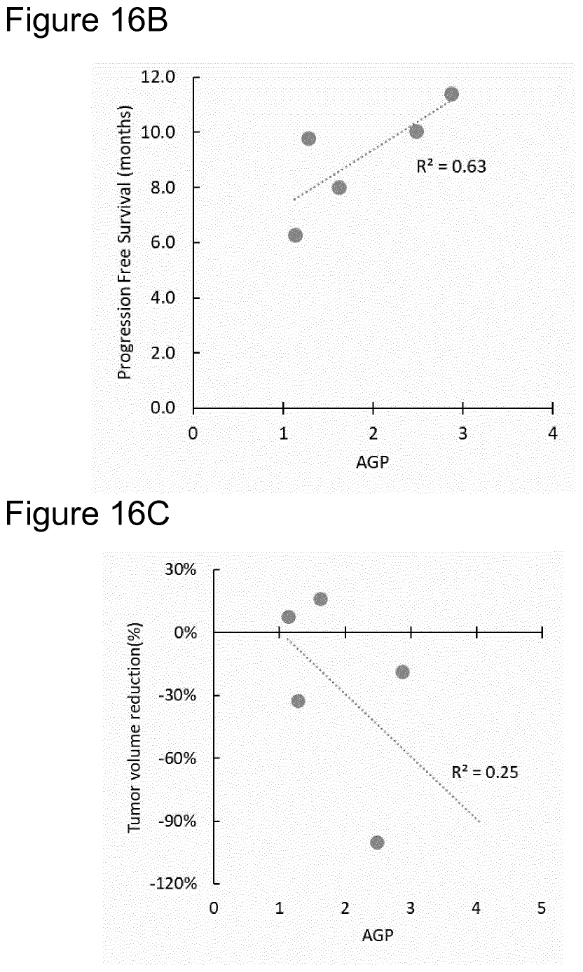

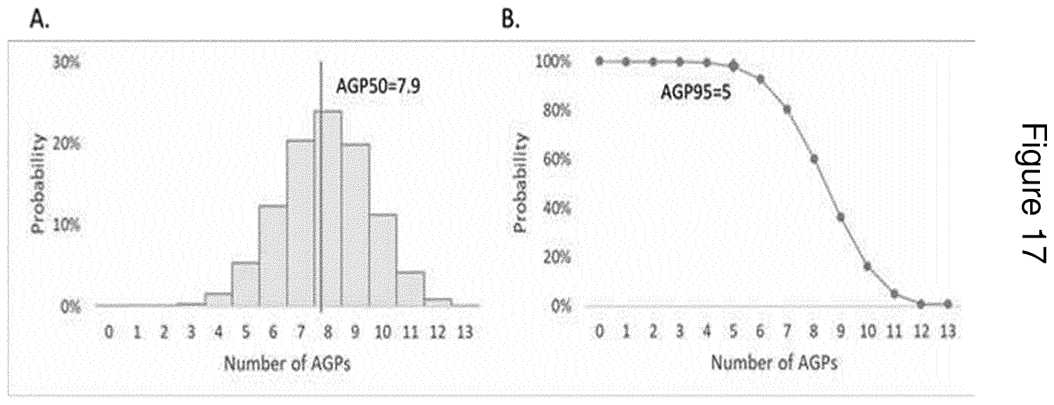

ROC analysis determined the diagnostic accuracy, using the PEPI3+ count as cut-off values (FIG. 2). The AUC value=0.73. For ROC analysis an AUC of 0.7 to 0.8 is generally considered as fair diagnostic value.

A PEPI3+ count of at least 1 (≥1 PEPI3+) best predicted a CTL response in the test dataset (Table 9). This result confirmed the threshold determined during the training (Table 6).

TABLE 9
Confirmation of the ≥1 PEPI3+ threshold to predict
likely CTL responders in the test/validation dataset.

| | PEPI3+ Count | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Sensitivity: | 0.75 | 0.52 | 0.26 | 0.23 | 0.15 | 0.13 | 0.08 | 0.05 | 0 | 0 | 0 | 0 |
| 1-Specificity: | 0.45 | 0.15 | 0.05 | 0 | 0 | 0 | 0 | 0 | 0 | 0 | 0 | 0 |

### Example 4—Clinical Validation of the ≥1 PEPI3+ Threshold as Novel Biomarker for PEPI Test

The PEPI3+ biomarker-based vaccine design has been tested first time in a phase I clinical trial in metastatic colorectal cancer (mCRC) patients in the OBERTO phase I/II clinical trial (NCT03391232). In this study, we evaluated the safety, tolerability and immunogenicity of a single or multiple dose(s) of PolyPEPI1018 as an add-on to maintenance therapy in subjects with mCRC. PolyPEPI1018 is a peptide vaccine containing 12 unique epitopes derived from 7 conserved TSAs frequently expressed in mCRC (WO2018158455 A1). These epitopes were designed to bind to at least three autologous HLA alleles that are more likely to induce T-cell responses than epitopes presented by a single HLA (See Examples 2 & 3). mCRC patients in the first line setting received the vaccine (dose: 0.2 mg/peptide) just after the transition to maintenance therapy with a fluoropyrimidine and bevacizumab. Vaccine-specific T-cell responses were first predicted by identification of PEPI3+-s in silico (using the patient's complete HLA genotype and antigen expression rate specifically for CRC) and then measured by ELISpot after one cycle of vaccination (phase I part of the trial).

Seventy datasets from 10 patients (Phase 1 cohort and dataset of OBERTO trial) was used to prospectively validate that PEPI3+ biomarker predicts antigen-specific CTL responses. For each dataset, predicted PEPI3+-s were determined in silico and compared to the vaccine-specific immune responses measured by ELISPOT assay from the patients' blood. Diagnostic characteristics (positive predictive value, negative predictive value, overall percent agreement) determined this way were then compared with the retrospective validation results described in Example 3.

The overall percent agreement was 64%, with high positive predictive value of 79%, representing 79% probability that the patient with predicted PEPI3+ will produce CD8 T cell specific immune response against the analyzed antigen. Clinical trial data were significantly correlated with the retrospective trial results (p=0.01) and provides evidence for the PEPI3+ calculation with PEPI test to predict antigen-specific T cell responses based on the complete HLA-genotype of patients (Table 10).

TABLE 10
Prospective validation of the ≥1 PEPI3+ and PEPI test

| | | Retrospective | Prospective |
|---|---|---|---|
| | | validation | validation (OBERTO) |
| Parameter | Definition | n = 81* | n = 70** |
| PPV | The likelihood that an individual with | 84% | 79% |
| Positive Predictive | a positive PEPI test result has antigen- | | |
| Value | specific T cell responses | | |
| NPV | The likelihood that an individual with | 42% | 51% |
| Negative Predictive | a negative PEPI test result does not | | |
| Value | have antigen-specific T cell responses | | |
| OPA | The percentage of results that are true | 70% | 64% |
| Overall Percent | results, whether positive or negative | | |
| Agreement | | | |
| Fisher's exact | | 0.01 | 0.01 |
| probability test (p) | | | |

*51 patients; 6 clinical trials; 81 dataset
**10 patients; Treos phase I clinical trial (OBERTO); 70 datasets

### Example 5—HLA Class I Genotype is Predictive for Risk of Melanoma (HLAT Score Based Method)

### Selection of Putative Immune Protective Tumor Antigens

It is hypothesized that tumor specific antigens (TSAs) are immune-protective antigens because cancer patients with spontaneous TSA specific T cell responses have favourable clinical course. 48 TSAs expressed in different tumor types were selected to study protective tumor specific T cell responses (Table 11). These TSAs have been studied in melanoma and other cancers and showed to induce spontaneous T cell responses.

TABLE 11
Selected TSAs for risk analysis

| Antigen | Indications |
|---|---|
| SPAG9 | CRC, RCC |
| AKAP4 | CRC |
| BORIS | Melanoma, CRC, HNSCC |
| Survivin | Melanoma, Lung(NSCLC), CRC, Bladder, Glioma, RCC |
| MAGE-A1 | Melanoma, Lung(NSCLC), CRC, Bladder, Glioma, HNSCC, RCC |
| PRAME | Melanoma, Lung(NSCLC), Bladder, HNSCC, RCC |
| CT45 | Melanoma, Lung(NSCLC), CRC, Glioma, HNSCC |
| NY-SAR-35 | Melanoma, Lung(NSCLC), Bladder |
| FSIP1 | Bladder |
| HOM-TES-85 | Lung(NSCLC), HNSCC |
| NY-BR-1 | Breast Cancer |
| MAGE-A9 | Melanoma, Bladder, RCC, HNSCC |
| SCP-1 | Melanoma, Lung(NSCLC), CRC, Bladder, Glioma, HNSCC |
| MAGE-A12 | Melanoma, Bladder, Glioma, HNSCC, RCC |
| MAGE-A10 | Melanoma, CRC, Bladder, Glioma, HNSCC |
| GATA-3 | RCC, HNSCC |
| GAGE-7 | Melanoma, CRC |
| SSX-4 | Melanoma, Lung (NSCLC), CRC, Bladder, Glioma, HNSCC |
| SPANXC | Melanoma, Lung(NSCLC), CRC, Bladder, HNSCC |
| CT46 | Bladder |
| MAGE-A3 | Melanoma, Lung(NSCLC), CRC, Bladder, Glioma, HNSCC, RCC |
| MAGE-C2 | Melanoma, Lung(NSCLC), CRC, Bladder, Glioma, HNSCC |
| TSP50 | Lung(NSCLC), CRC |
| EpCAM | Lung(NSCLC), CRC, Bladder, RCC |
| CAGE | Lung(NSCLC), CRC, HNSCC |
| MAGE-A8 | Melanoma, CRC, Bladder |
| FBXO39 | Lung(NSCLC), CRC, RCC |
| PAGE-4 | Lung(NSCLC) |
| MAGE-A6 | Melanoma, CRC, Bladder, RCC |
| BAGE-4 | CRC, Bladder, Glioma, HNSCC |
| MAGE-C1 | Melanoma, Lung(NSCLC), CRC, Bladder, HNSCC |
| NY-ESO-1 | Melanoma, Lung(NSCLC), CRC, Bladder, Glioma, HNSCC, RCC |
| MAGE-A2 | Melanoma, CRC, Bladder, Glioma, HNSCC, RCC |
| XAGE-1 | Melanoma, Lung(NSCLC), Glioma, HNSCC |
| MAGE-A11 | Melanoma, Glioma, HNSCC |
| SSX-2 | Melanoma, Lung(NSCLC), CRC, Bladder, Glioma, HNSCC |
| LAGE-1 | Melanoma, Lung(NSCLC), CRC, Bladder, Glioma, HNSCC |
| MAGE-A4 | Melanoma, Lung(NSCLC), CRC, Bladder, Glioma, HNSCC, RCC |
| MAGE-A5 | Melanoma, Lung(NSCLC), CRC, HNSCC |
| MAGE-B2 | Melanoma, Lung(NSCLC), HNSCC |
| MAGE-B1 | Melanoma, Lung |
| HAGE | Melanoma, Lung(NSCLC), CRC, Bladder, HNSCC, RCC |
| SSX-1 | Melanoma, Lung(NSCLC), CRC, Bladder, Glioma, HNSCC |
| NXF2 | Melanoma, Lung(NSCLC), CRC, Bladder, HNSCC |
| SAGE | Melanoma, Lung(NSCLC), Bladder, Glioma, HNSCC |
| LEMD1 | CRC, Glioma |
| OY-TES-1 | Lung(NSCLC), CRC, Bladder |
| LDHC | Melanoma, Lung(NSCLC), Glioma |

CRC: colorectal cancer,
NSCLC: non-small cell lung cancer,
HNSCC: head&neck squamous cell carcinoma,
RCC: renal cell carcinoma

Incidence Rate for Melanoma Correlates with HLAT Number Indicating the Breadth of Melanoma Specific T Cell Responses

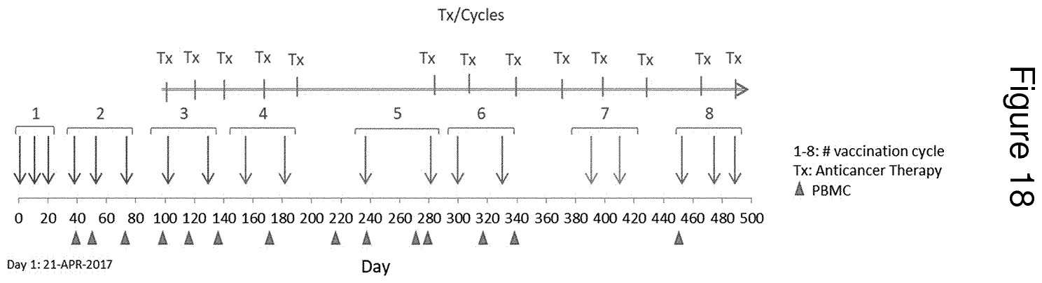

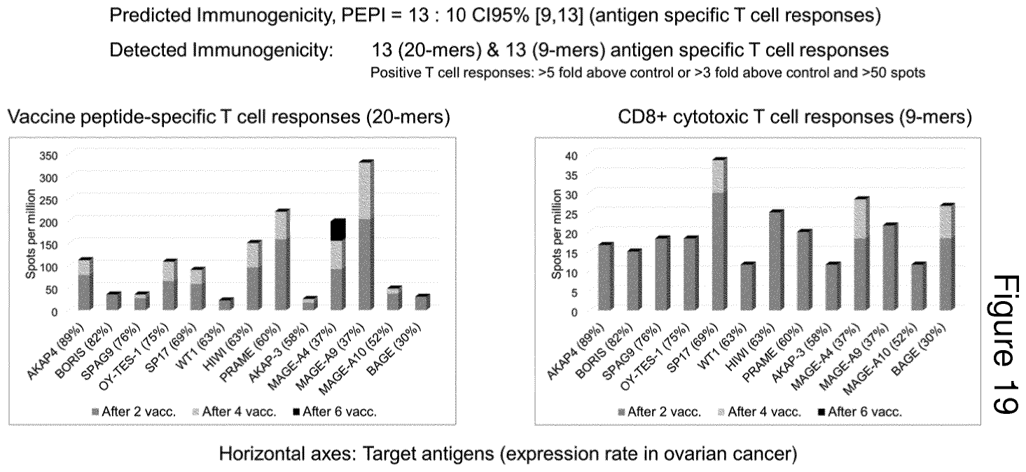

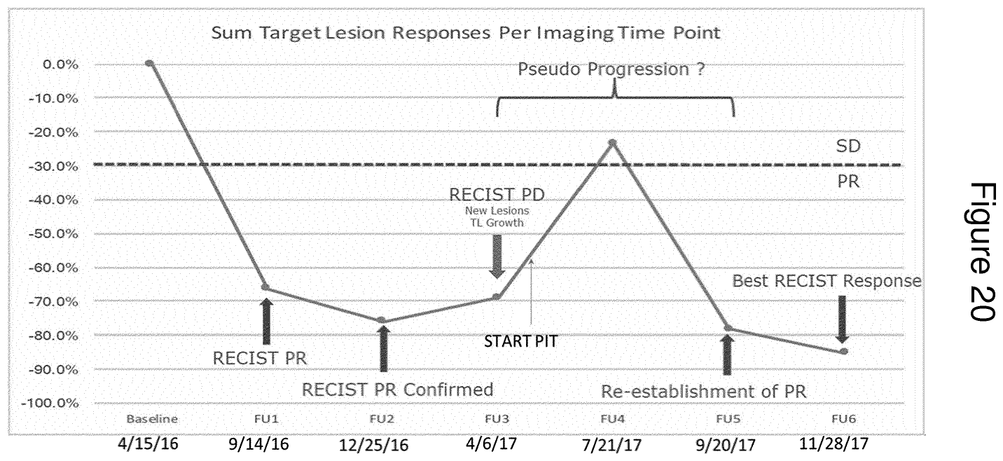

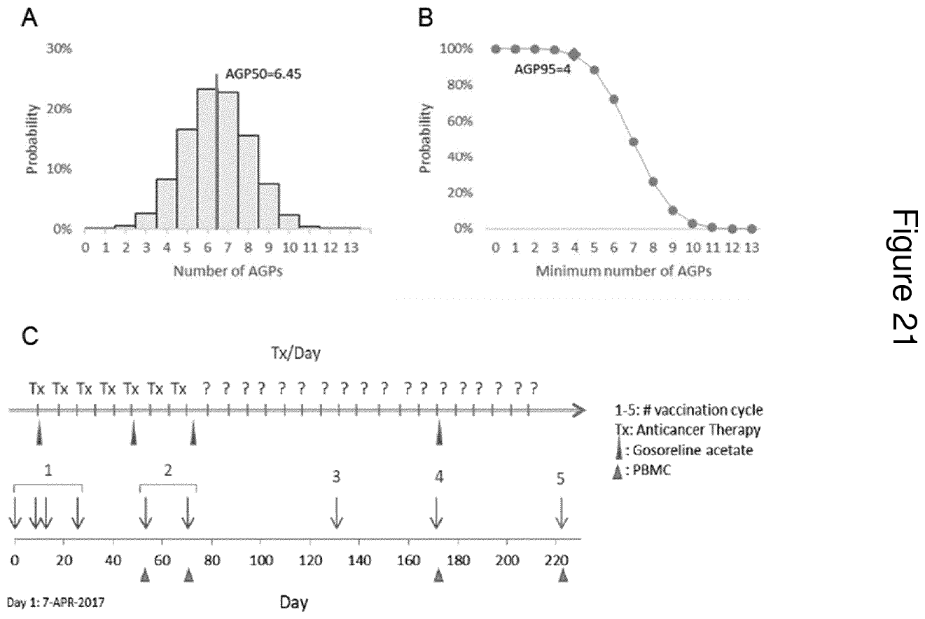

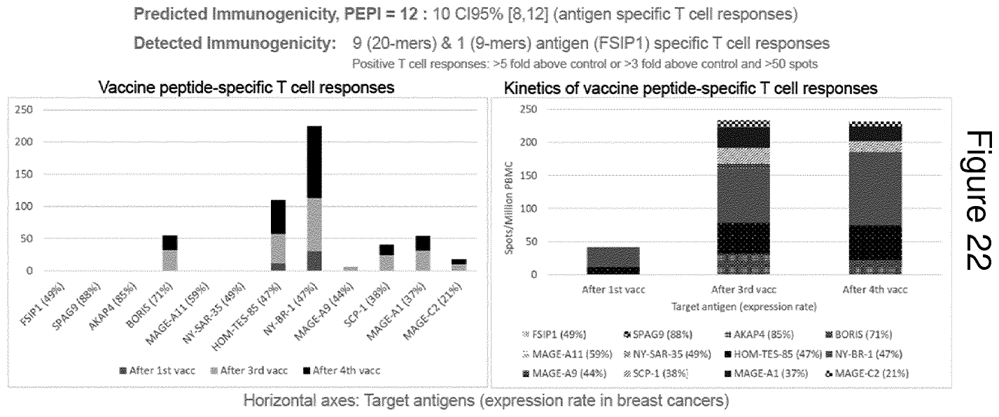

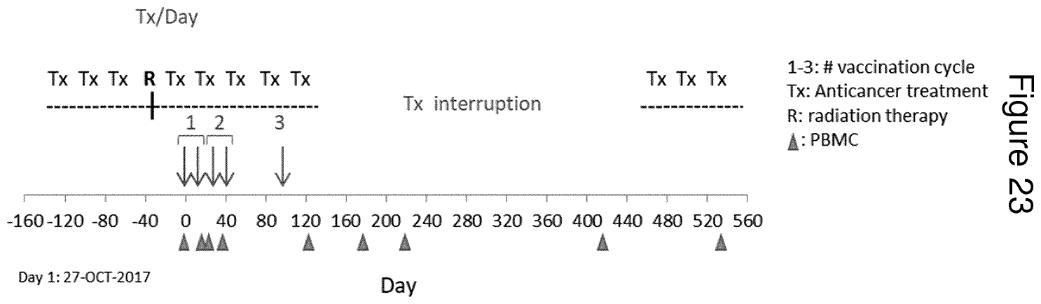

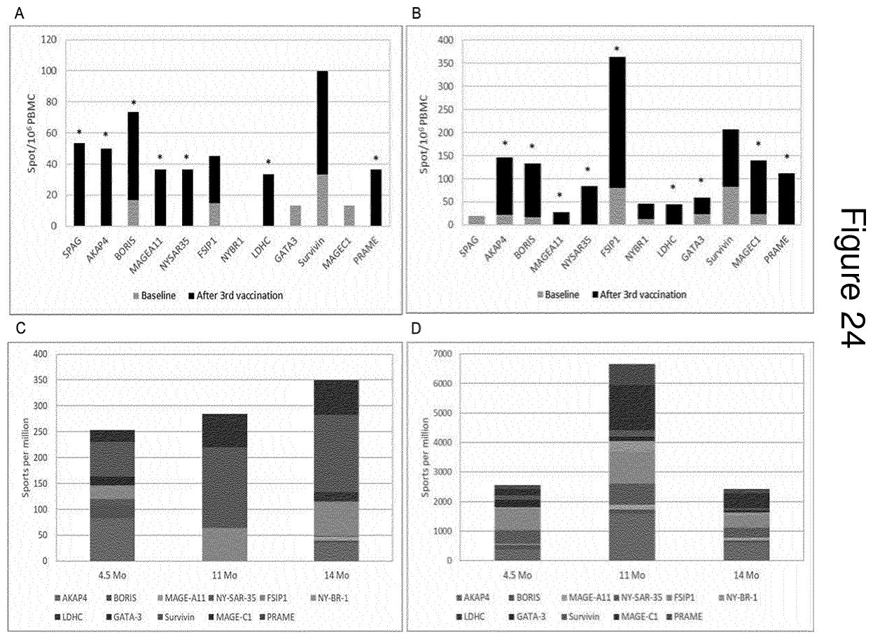

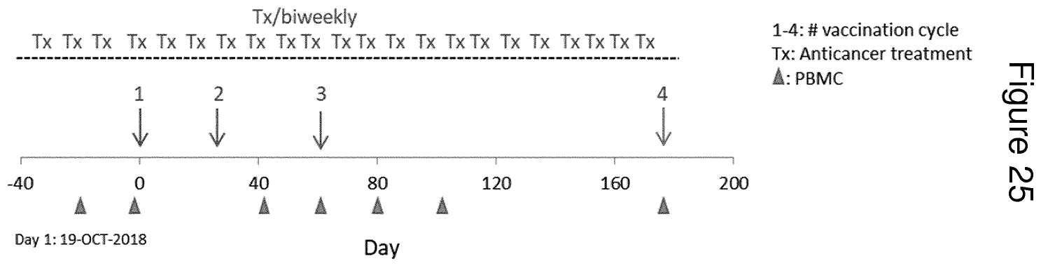

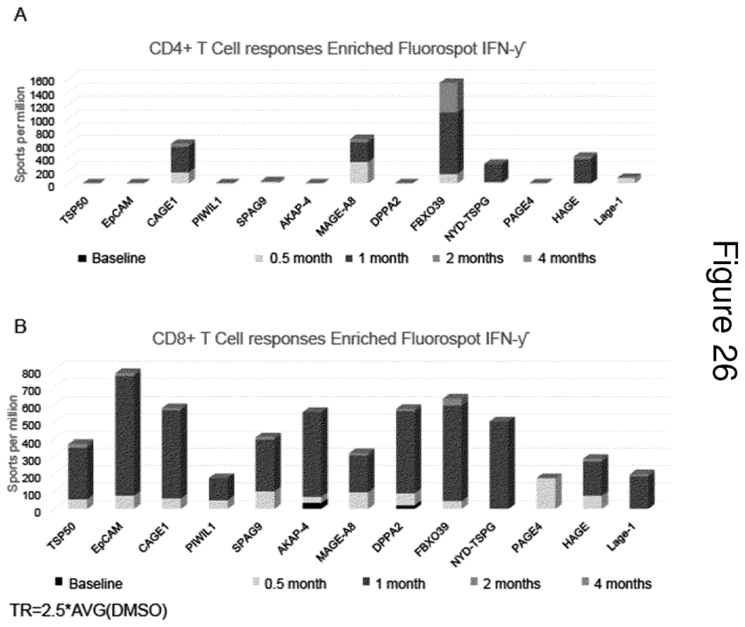

It is hypothesized that the HLAT number for the 48 TSAs in a population where melanoma has high incidence rate would be lower than in a population with high incidence rate. To show this the HLAT number for the 48 TSAs was determined in different ethnic populations for which melanoma incidence are available (FIG. 3).

Subjects in the far East Asian/Pacific region were found to have much higher HLAT numbers than subjects of European or US origin (FIG. 3). For example the incidence rate of melanoma is 1.5 per 100,000 persons in both Taiwan and Asia-Pacific islanders in the US, which is significantly lower than overall in the US (21.1 per 100,000 per year).

HLAT Scores (s) are in agreement with the incidence rate of melanoma in different countries (FIG. 4). 20 data points were obtained to compute the average HLAT Score and incidence rates (incidence rates were available by countries, HLA data were available by ethnics, therefore paired observations could only be obtained for those countries that have a dominating ethnicity). FIG. 4 shows the significant difference between the incidence rates in countries where the average HLAT Score is less than 75 and the incidence rates in countries where the average HLAT Score is higher than 75. These results suggest that HLA genotypes of subjects influence the incidence of melanoma in different ethnic populations and show that the HLAT numbers could estimate a subject's melanoma specific T cell responses.

### HLAT Score of a Subject is an HLA Genotype Linked Risk Factor for Developing Melanoma

HLAT numbers predicted the breadth of T cell responses against 48 selected TSAs. It is hypothesized that not all the HLATs of a subject play equally important role in the immunological control of melanoma. Therefore, the HLATs (for the 48 TSAs) were weighted based on capacity to separate melanoma patients from a general population. In general, the larger the weight, the more important is the corresponding TSA. Indeed, the AUC was already above 0.6 using the initial weights (truncated log p-values).

Performance of a Binary Classifier at Separating Melanoma Patients from the Background

This study compared a US subpopulation (n=1400) from the dbMHC dataset (7,189 patient cohort) to melanoma subjects, also with US origin (n=513) using a binary classifier (see Methods). FIG. 5 shows the ROC curve achieved using the HLAT Score as a binary classifier. The HLAT Score predicts which of the two possible groups a subject belongs to: melanoma cancer group or background population. The ROC curve is presented by plotting the true positive rate (TPR) against the false positive rate (FPR) at various HLAT Score threshold settings.

The AUC value obtained was 0.645. This value indicates a significant separation between two groups, in particular because in the case of melanoma/cancer incidence there is not only a single cause (e.g. HLAT) of discrimination. Most remarkably sun and indoor tanning exposure is a significant determinant of melanoma risk, as are phenotypes such as blond or red hair, blue eyes and freckles and genetic factors such as the high penetrance, 3 medium penetrance and 16 low penetrance genes associated to melanoma described by Read et al. (J. Med. Genet. 2016; 53(1): 1-14). Indeed, the transformed z score of 10.065 achieved in the present study is highly significant (p<0.001).

### HLAT Score of a Subject is an HLA Genotype Linked Risk Factor for Developing Melanoma

The total test population (background population mixed with cancer population) was divided into five equally large groups based on HLAT Score. The Relative Immunological Risk (RiR) in each group was determined compared to the risk in an average US population (FIG. 6). For example, the risk of developing melanoma in the first subpopulation is 4.4%, while the US average is 2.6%, therefore, this subgroup has a 1.7 relative immunological risk. The group with the lowest HLAT Score represents the population with the highest immunological risk of developing cancer. The group with the highest HLAT Score represent the population with the lowest immunological risk of developing cancer. The most risky subpopulation consists of those subjects that have HLAT Score smaller than 26. The HLAT Score varies between 29 and 51 in the second most risky subpopulation. In the middle 20% are those subjects whose HLAT Score is greater or equal than 51 and lower than 88 and the RiR<1, suggesting that certain HLAT Scores are associated with reduction of melanoma risk. Interestingly, this HLAT Score range of 51-88 is similar to the HLAT Score (75) which could separate populations with low and high incidence rate for melanoma (FIG. 6). In the second most protected subpopulation, the HLAT Score is between 88 and 164. Finally, in the most protected subpopulation, each subject has a HLAT Score of at least 164. In these subpopulations, the relative immunological risk of developing melanoma is monotonously decreasing as shown in FIG. 6. Although there is no significant change between consecutive groups, the difference between the first and the last group is significant (p=0.001).

### Example 6—HLA Class I Genotype is Predictive of Risk of Different Types of Cancer (HLAT Score Based Method)

A similar analysis was performed for six other cancer indications. The results are summarised in Table 12. The AUC values were significant for melanoma, lung cancer, renal cell carcinoma, colorectal cancer and bladder cancer. The p value is not significant for head and neck cancer. However, head and neck cancer is associated with viral HPV infection. Only TSAs were used in the present study, no viral proteins were included. It may be that the risk of developing certain cancers, such as head and neck cancer, that can be associated with viral infections could better be determined by including viral antigens in the analysis.

TABLE 12
Summary of the immunological risk prediction for different
types of cancers compared to an average population

| | | RiR | | | | |
|---|---|---|---|---|---|---|
| Cancer | Cohort | Risk | Protected | RiR | | |
| type | Size | Group* | Group* | Ratio | AUC | p |
| Melanoma | 513 | 1.73 | 0.31 | 5.53 | 0.65 | <0.001 |
| Lung (non- | 370 | 1.46 | 0.54 | 2.72 | 0.61 | <0.001 |
| small cell) | | | | | | |
| Renal cell | 129 | 1.59 | 0.58 | 2.74 | 0.60 | <0.001 |
| Colorectal | 121 | 1.83 | 0.61 | 2.97 | 0.62 | <0.001 |
| Bladder | 87 | 1.77 | 0.63 | 2.80 | 0.61 | <0.001 |
| Glioma | 82 | 1.36 | 0.79 | 1.71 | 0.57 | 0.017 |
| Head and neck | 58 | 1.02 | 0.17 | 5.90 | 0.54 | 0.15 |

By dividing the test population (background population mixed with cancer population) into five equally large subgroups based on the HLAT Scores, we could calculate the relative immunological risk associated with certain HLAT Scores in case of non-small cell lung cancer, renal cell carcinoma and colorectal cancer (FIGS. 7A-C). For other indications, the number of cancer subjects in a subpopulation was too small to perform similar analysis.

The relative immunological risk ratio was calculated between the Risk subgroup (20% of the test population with the lowest HLAT Score) and the Protected subgroup (20% of the test population with the highest HLAT Score) compared to the risk in an average US population. For example, the risk of developing melanoma in the characterized riskiest subpopulation is 4.4%. The US average is 2.4%, therefore, the Risk group has a 1.7 relative immunological risk. The risk of developing melanoma in the Protected group is 0.7%. That is, the relative immunological risk of the most protected group is 0.31. In other words, this group has more than three times lower risk to develop melanoma compared to the average population. The risk ratio achieved for melanoma is 5.53 (Table 12).

### Methods for Examples 5, 6 and 10

### HLA Genotype Data of Subjects in a General Population

7,189 eligible subjects with complete 4-digit HLA genotype were identified from dbMHC database. The ethnicity of each subject was indicated. Our analysis revealed that the HLA background of subpopulations coming from different geographic regions differ considerably. To eliminate this geographic effect, we selected the American subpopulation (1400 subjects) as a background (healthy) population, and the HLA sets of this subgroup were compared to the HLA sets of geographically/ethnically matched cancer subjects. The American subpopulation consists of all Caucasian, Hispanic, Asian-American, African-American and native ethnics.

### HLA Genotype Data of Cancer Patients

Eligible patients had complete 4-digit HLA class I genotype. Data from 513 patients with melanoma were obtained from the following sources:

429 melanoma subjects were available with complete 4-digit HLA class I genotype from 3 peer-reviewed publications (Snyder et al. N Engl J Med. 2014; 371(23):2189-99; Van Allen et al. Science. 2015; 350(6257):207-11; Chowell et al. Science. 2018; 359(6375):582-7). Patients were treated with anti-CTLA-4 and/or PD-1/PD-L1 inhibitors at the Memorial Sloan Kettering Cancer Center, New York (MSKCC). High-resolution HLA class I genotyping from normal DNA was performed using DNA sequencing data or clinically validated HLA typing assay by LabCorp. 17 stage III/IV melanoma patients' HLA genotype was kindly provided by MSKCC. These patients were treated with Ipilimumab at MSKCC, New York (Yuan et al. Proc Natl Acad Sci USA. 2011; 108(40):16723-8). 65 melanoma patients from a phase 3 randomized, double-blind, multicenter study (CA184007, NCT00135408) and a phase 2 (CA184002, NCT00094653) in patients with unresectable stage III or IV malignant melanoma and previously treated unresectable stage III or stage IV melanoma, correspondingly. These 65 patients treated at MSKCC, New York site had samples available for HLA testing which were kindly provided by Bristol-Myers-Squibb. Samples were retrospectively tested with NGS G group resolution and HLA allele interpretation was based on IMGT/HLA database version 3.15. HLA results were obtained using sequence based typing (SBT), sequence specific oligonucleotide probes (SSOP), and/or sequence specific primers (SSP) as needed to obtain the required resolution. The HLA testing was performed by LabCorp, USA.

HLA genotype data of 370 patients with non-small cell lung cancer, 129 renal cell carcinoma, 87 bladder cancer, 82 glioma and 58 head and neck cancer subjects were collected from peer reviewed publication (Chowell et al.).

Data from 37 colorectal cancer (CRC) patients' HLA genotype were obtained from the National Center for Biotechnology (NCBI) Sequence Read Archive, Encyclopedia of deoxyribonucleic acid elements (Boegel et al. Oncoimmunology. 2014; 3(8):e954893). Blood samples from 211 Vietnamese and 84 white, non-Hispanic CRC patients were obtained from Asterand Bioscience and HLA genotype were identified by LabCorp (Burlington N.C.).

### TSA Sequence Data

48 TSAs were selected. The amino acid sequence data of these antigens were obtained from UniProt.

### Incidence Rates

Incidence rates were obtained from globocan.iarc.fr/Pages/online.aspx,

### Human Leukocyte Antigen Triplets (HLATs)

HLA class I genes are expressed in most cells and bind to epitopes that are recognized by T cell receptors. Epitopes that bind to at least three HLAs (HLA triplet or HLAT) of a person's six HLA alleles can generate T cell responses. For each j=1, 2, . . . 6 we set up a scoring system to score the subjects' immune system based on how well they can bind epitopes. Based on combinatorics, there are

$$( \begin{matrix}k \\ j\end{matrix} ) = \frac{k !}{( k - j ) ! j !}$$

possible HLA allele j-sets for a particular epitope, where k is the number of autologous HLA alleles that can bind the epitope. When we are interested in HLA triplets, j=3. Therefore, HLAT number of a subject for an antigen is defined as the total sum of HLATs.

HLATs of subjects are identified with the PEPI test, validated to identify HLA binding epitopes with 93% accuracy.

### Immunogenetic Predictor: HLAT Score

The HLAT Score of a subject x is defined:

s(x)=Σ_{c∈C}w(c)p(x,c) (1)

where C is the set of the TSAs, c is a particular TSA, w(c) is the weight of TSA c, and p(x,c) is the HLAT number of the TSA c in subject x.

### HLAT Score Weight Optimization

The initial weight was 0 for each TSA whose HLAT Scores did not significantly separated cancer patients from the background population. Since we assumed that having HLATs do not increase the chance to develop cancer, only non-negative weights were considered. The initial weights were defined as

$$w ( c ) = max \{ 0 , log ( \frac{0.05}{48} ) - log ( t ( c ) ) \}$$

where t(c) denotes the p-value of the one sided t-test on the HLAT Score of the TSA c of the cancer and background populations and 48 is the Bonferroni correction.
The initial weights were further optimized using the Parallel Tempering. Six parallel Markov chains has been applied with temperatures RT=0.001, 0.01, 0.02, 0.04, 0.1, 0.2. The hypothetical energy was defined as −1 times the sum of the RiRR (Relative immunological risk ratio, see below) and AUC. The weights providing the largest relative risk ratio has been reported.

### Relative Immunological Risk (RiR)

RiR was calculated by the ratio of the risks between a subpopulation and the total test population (cancer population and background population) with the 95% confidence intervals (CI). For this purpose, the general population was assembled in that way to resemble the percentage of different cancer patients in a general US population taking into consideration the life-time risk. The lifetime risks of developing the different type of cancers was obtained from the website of the American Cancer Society. Typically, the lifetime risk of men and women differ, so we took the (harmonic) average of them. The so-obtained risks are: 1:38 for melanoma, 1:16 for lung cancer, 1:61 for renal cell carcinoma, 1:23 for colorectal cancer, 1:41 for bladder cancer, 1:55 for head and neck cancer and 1:161 for glioma. RiR>1 indicates that subjects have higher risk of developing a certain cancer compared to subjects in an average population.

### RiR Ratio (RiRR)

RiR Ratio was calculated as the ratio between the groups with the highest and lowest HLAT Scores.

### Example 7—HLA-Score Based on HLA Triplets Provide the Best Separation Between Cancer and Background Subjects

When developing a screening test, we considered several scoring schemes. The potential scoring schemes differ in the minimum size of HLA allele sets binding to one particular epitope that is considered to contribute to the score of a subject. For each size of HLA allele subsets j=1, 2, . . . , 6, we computed the significance scores for each allele based on how frequently it participates in HLA j-tuples of the training subjects binding to a particular epitope. Briefly, we considered the significance score positive, if subjects with a given HLA allele had significantly more epitopes with HLA j-mers than subjects without the given HLA allele. The significance score was negative if the subjects with the given HLA allele had significantly less epitopes with HLA j-mers than the subjects without the given HLA allele. Then for each subject we summed the significance scores of his/her HLA alleles. Next, we tested how well these summed scores can distinguish melanoma and background subjects by computing the area under the receiver operating characteristic curve (ROC-AUC, AUC). According to Table 13, the best separation of melanoma and background population was achieved equally for j=2 and j=3. The remarkable difference between the AUC values for the different scores based on 1-set versus j-sets, j>1, suggest that presentation of an epitope by multiple HLA alleles could play an important role in developing efficient anti-tumor immune response. Furthermore, these results suggest that separation of cancer and background (healthy) subjects based on single allele of their HLA genotype would be challenging. The drop-off in the AUC values when j=6 can be explained with the fact that there are only a very limited number of epitope-HLA allele combinations where all the 6 HLA alleles of a subject can bind the epitope.

TABLE 13
The AUC values computed for melanoma with different HLA j-sets

| j | AUC |
|---|---|
| 1-set | 0.60 |
| 2-set | 0.69 |
| 3-set | 0.69 |
| 4-set | 0.68 |
| 5-set | 0.68 |
| 6-set | 0.61 |

### Example-8—HLA-Score is a Risk or Protective Indicator of Melanoma, with Explanations of RiR and RiRR

The AUC value (0.69) comparing US melanoma and background subjects indicates significant separation between the two groups, using the HLA-score. Indeed, the transformed z score was 12.57, which was highly significant (p<0.001). These results demonstrate that subjects' HLA genotype influence the genetic risk for developing melanoma. Based on the HLA-score, the background and melanoma populations were divided into five equal-size subgroups based on their HLA-score (s); s<34, 34≤s<55, 55≤s<76, 76≤s<96 and 96<s. The Relative Risk (RR) of each subgroup was computed (FIG. 8). We found that subjects with the highest immunological risk of developing melanoma (6.1%) are in the lowest HLA-score subgroup (s<34). Since the average risk of melanoma in the USA is 2.6%, a subject in the s<34 subgroup has 2.3 fold higher risk for melanoma than an average USA subject. In contrast, the subgroup with the highest HLA-score (96<s) represents subjects with the lowest immunological risk of developing melanoma (1.1%). A subject in this subgroup has 0.42 fold lower risk than an average subject in the USA. Differences between the first and the last subgroup was significant (p<0.05).

We computed the risk ratio between the most protected and most at-risk groups (RRₑₓₜᵣₑₘᵢₜᵢₑₛ). We found that the RRₑₓₜᵣₑₘᵢₜᵢₑₛ for melanoma is 5.69 indicating that subjects with HLA-score less than 34 have approximately 6 fold higher risk of developing melanoma compared to subjects with HLA-score higher than 96 (Table 14).

### Example 9—Performance of the HLA-Score as Predictor of the Risk for Developing Different Types of Cancers

In some cases the significance score of an HLA allele (h) is defined as

$$s ( h ) \text{:=} sign ( h ) max \{ 0 , log ( \frac{0.05}{B} ) - log ( u ( h ) ) \}$$

where u(h) is the p-value of the two-sided u-test for allele h determining whether or not the number of HLATs are different in two subsets of individuals: one subset in which the individuals have HLA h, and one subset in which the individuals do not have HLA h. B is the Bonferroni correction, and sign(h) is +1 if the average number of HLATs is larger in the subpopulation having the h allele than in the subpopulation not having h, and −1 otherwise. In some cases, this initial score may be further optimized using any suitable method as known to those skilled in the art. In some cases the sum of these significance scores is used to determine the risk that the subject will develop cancer correlates to the risk that the subject will develop cancer.

The concrete score to be used depends on the indication and the a priori data. In some cases, the choice will be made based on the performance of the different computations on available test datasets. The performance might be evaluated by the AUC value (the area under the ROC curve) or by any other goodness of performance score known by those skilled in the art.

We determined the ROC curve, RR and RRₑₓₜᵣₑₘᵢₜᵢₑₛ for non-small cell lung, renal cell, colorectal, bladder, head and neck cancers and glioma using the same methods described for melanoma (Table 14). The ROC-AUC values were significant for all cancer types, except for colorectal cancer.

We obtained a RRₑₓₜᵣₑₘᵢₜᵢₑₛ range of 2.35-5.69 for the studied cancer indications, suggesting different levels of immune protection against different types of cancer (Table 14). However, RRₑₓₜᵣₑₘᵢₜᵢₑₛ>2 for all cancer indications demonstrate that HLA genotype represents a substantial genetic risk of developing cancer.

TABLE 14
Immunological risk prediction in different cancer types

| | Co- | RR | | | | |
|---|---|---|---|---|---|---|
| Cancer | hort | Risk | Protected | | | |
| type | Size | Group* | Group* | RRₑₓₜᵣₑₘᵢₜᵢₑₛ | AUC | p |
| Melanoma | 513 | 2.34 | 0.41 | 5.69 | 0.69 | <0.001 |
| Lung (non- | 370 | 1.84 | 0.41 | 4.49 | 0.66 | <0.001 |
| small cell) | | | | | | |
| Renal cell | 129 | 1.73 | 0.51 | 3.41 | 0.63 | <0.001 |
| Colorectal | 121 | 1.28 | 0.55 | 2.35 | 0.55 | 0.008 |
| Bladder | 87 | 1.89 | 0.46 | 4.14 | 0.66 | <0.001 |
| Glioma | 82 | 1.83 | 0.48 | 3.81 | 0.63 | <0.001 |
| Head and | 58 | 1.21 | 0.51 | 2.38 | 0.62 | 0.001 |
| neck | | | | | | |

*Risk Group, the 20% of the general population with the lowest HLA-score; Protected Group, the 20% of the general population with the highest HLA-score. Each cancer indication was classified against the same background population. RRₑₓₜᵣₑₘᵢₜᵢₑₛ is the risk ratio of the most at-risk and most protected groups; AUC, area under the ROC curve. Bonferroni corrected p value smaller than 0.007 demonstrate significance.

### Example 10—Risk Screening for Patient-D for CRC and Vaccine Design

This example shows how to compute the HLAT Score of Patient-D described in Example 20. Patient-D has been diagnosed with metastatic colorectal cancer. Using patient-D's HLA genotype the predicted number of PEPI3, PEPI4, PEPI5 and PEPI6 epitopes on the 48 selected TSAs were determined (Table 15). Based on the statistics, the total number of HLATs for each TSA were computed (lines 6, 14 and 22 of Table 15) and the weighted scores for each TSA (lines 8, 16 and 24 of Table 15). This weighted score is simply the product of the total number of HLATs and the weights of the TSAs (lines 7, 15 and 23 of Table 15). The weights were obtained with the method described in the “HLAT Score Weight Optimization” section of Example 6. The summed weighted score (as described in Equation (1)) is 43.09. Based on the comparison of American CRC and American background population, Patient-D has a 1.26-fold risk to develop colorectal cancer than an average person in the USA. Since the risk for developing CRC in the USA is 4.2%, the risk for Patient-D based on our result is 5.3%.

TABLE 15

| Antigen | SPAG9 | AKAP4 | BORIS | Survivin | MAGE-A11 | PRAME | CT45 | NY-SAR-35 | FSIP1 |
|---|---|---|---|---|---|---|---|---|---|
| #PEPI3 | 5 | 6 | 0 | 0 | 3 | 6 | 0 | 2 | 0 |
| #PEPI4 | 0 | 0 | 0 | 0 | 0 | 4 | 0 | 0 | 0 |
| #PEPI5 | 0 | 1 | 0 | 0 | 0 | 2 | 0 | 0 | 0 |
| #PEPI6 | 0 | 0 | 0 | 0 | 0 | 0 | 0 | 0 | 0 |
| Total HLAT | 5 | 16 | 0 | 0 | 3 | 42 | 0 | 2 | 0 |
| Weights | 0.05453 | 0.00086 | 0.48840 | 3.32572 | 2.02242 | 0.08729 | 0.12114 | 0.00360 | 0.13681 |
| Weighted Scores | 0.27267 | 0.01380 | 0 | 0 | 6.06728 | 3.66633 | 0 | 0.00720 | 0 |

| Antigen | HOM-TES-85 | NY-BR-1 | MAGE-A9 | SCP-1 | MAGE-A1 | MAGE-A10 | GAGE-7 |
|---|---|---|---|---|---|---|---|
| #PEPI3 | 0 | 3 | 2 | 1 | 2 | 3 | 0 |
| #PEPI4 | 0 | 0 | 0 | 0 | 0 | 0 | 0 |
| #PEPI5 | 0 | 0 | 0 | 0 | 0 | 0 | 0 |
| #PEPI6 | 0 | 0 | 0 | 0 | 0 | 0 | 0 |
| Total HLAT | 0 | 3 | 2 | 1 | 2 | 3 | 25 |
| Weights | 2.20805 | 0.02666 | 0.42470 | 1.48437 | 0.05817 | 0.63445 | 0.03178 |
| Weighted Scores | 0 | 0.07997 | 0.84941 | 1.48437 | 0.11635 | 1.90337 | 0.79450 |

| Antigen | SSX-4 | SPANXC | CT46 | MAGE-A3 | MAGE-C2 | TSP50 | EpCAM | CAGE | MAGE-A8 |
|---|---|---|---|---|---|---|---|---|---|
| #PEPI3 | 0 | 0 | 2 | 1 | 2 | 1 | 0 | 2 | 2 |
| #PEPI4 | 0 | 0 | 0 | 0 | 0 | 0 | 2 | 0 | 0 |
| #PEPI5 | 0 | 0 | 0 | 0 | 0 | 0 | 0 | 0 | 0 |
| #PEPI6 | 0 | 0 | 0 | 0 | 0 | 0 | 0 | 0 | 0 |
| Total HLAT | 0 | 0 | 2 | 1 | 2 | 1 | 8 | 2 | 2 |
| Weights | 0.29159 | 1.36866 | 0.72798 | 2.00253 | 0.23195 | 0.02072 | 0.31689 | 0.04891 | 1.13916 |
| Weighted Scores | 0 | 0 | 1.45597 | 2.00253 | 0.46391 | 0.02072 | 2.53515 | 0.09782 | 2.27833 |

| Antigen | FBXO39 | PAGE-4 | MAGE-A6 | BAGE-4 | MAGE-C1 | NY-ESO-1 | MAGE-A2 |
|---|---|---|---|---|---|---|---|
| #PEPI3 | 5 | 1 | 2 | 0 | 3 | 1 | 1 |
| #PEPI4 | 1 | 0 | 0 | 0 | 0 | 0 | 1 |
| #PEPI5 | 0 | 0 | 0 | 0 | 0 | 1 | 0 |
| #PEPI6 | 0 | 0 | 0 | 0 | 0 | 0 | 0 |
| Total HLAT | 9 | 1 | 2 | 0 | 3 | 11 | 5 |
| Weights | 0.01892 | 2.92234 | 0.91159 | 3.14030 | 0.01183 | 0.01775 | 0.23611 |
| Weighted Scores | 0.17030 | 2.92234 | 1.82318 | 0 | 0.0355 | 0.19531 | 1.18056 |

| Antigen | XAGE-1 | MAGE-A12 | SSX-2 | LAGE-1 | MAGE-A4 | MAGE-A5 | MAGE-B2 |
|---|---|---|---|---|---|---|---|
| #PEPI3 | 0 | 1 | 0 | 1 | 0 | 0 | 0 |
| #PEPI4 | 0 | 0 | 0 | 0 | 0 | 0 | 1 |
| #PEPI5 | 0 | 0 | 0 | 0 | 0 | 0 | 0 |
| #PEPI6 | 0 | 0 | 0 | 0 | 0 | 0 | 0 |
| Total HLAT | 0 | 1 | 0 | 1 | 0 | 0 | 4 |
| Weights | 14.00 | 0.0231 | 1.5775 | 0.0725 | 0.4303 | 1.6607 | 0.1694 |
| Weighted Scores | | 0.0231 | | 0.0725 | | | 0.6778 |

| Antigen | MAGE-B1 | HAGE | SSX-1 | NXF2 | SAGE | LEMD1 | OY-TES-1 | LDHC |
|---|---|---|---|---|---|---|---|---|
| #PEPI3 | 0 | 0 | 0 | 0 | 1 | 0 | 1 | 3 |
| #PEPI4 | 0 | 1 | 0 | 2 | 1 | 0 | 2 | 0 |
| #PEPI5 | 0 | 1 | 0 | 0 | 0 | 0 | 0 | 0 |
| #PEPI6 | 0 | 0 | 0 | 0 | 0 | 0 | 0 | 0 |
| Total HLAT | 0 | 14 | 0 | 8 | 5 | 0 | 9 | 3 |
| Weights | 0.6205 | 0.0258 | 0.1048 | 1.3551 | 0.0164 | 1.1897 | 0.0495 | 0.0496 |
| Weighted Scores | | 0.3614 | | 10.841 | 0.0821 | | 0.4459 | 0.1489 |

### Example 11—CRC Phase I Trial Results: PEPI Vs HLAT Vs Immunogenicity

In the OBERTO trial, we predicted immune response for 7 antigens and 11 subjects, and also measured immune responses in 10 patients' specimen. The 7 antigens of the vaccine are part of the 48 TSAs. The predictions and measurements are summarized in Table 16. The overall percentage agreement is 64%.

TABLE 16
Measured and PEPI test predicted immune responses for the
vaccine-comprising peptides specific for the listed TSAs.

| Patient/ | 10002 | | 10003 | | 10004 | | 10005 | | 10007 | |
|---|---|---|---|---|---|---|---|---|---|---|
| AG | Mea. | Pred. | Mea. | Pred. | Mea. | Pred. | Mea. | Pred. | Mea. | Pred. |
| TSP50 | − | − | − | − | + | − | − | − | + | + |
| EpCAM | + | + | − | − | + | + | + | + | + | − |
| Survivin | + | + | − | − | + | + | − | − | + | − |
| MAGE-A8 | + | − | − | − | − | − | − | − | + | − |
| CAGE1 | + | + | − | − | + | + | − | + | + | − |
| SPAG9 | − | − | − | + | − | − | − | − | + | − |
| FBXO39 | − | + | − | + | − | − | + | − | + | + |

| | | | | | |
|---|---|---|---|---|---|
| OPA: | 71% | 71% | 86% | 71% | 29% |

| | |
|---|---|
| OPA Full: | 64% |

| | | | | | |
|---|---|---|---|---|---|
| Patient/ | 10008 | 20001 | 20002 | 20003 | 20004 |

| AG | Mea. | Pred. | Mea. | Pred. | Mea. | Pred. | Mea. | Pred. | Mea. | Pred. |
|---|---|---|---|---|---|---|---|---|---|---|
| TSP50 | + | + | + | + | + | − | + | + | + | + |
| EpCAM | + | + | + | + | + | − | + | − | + | + |
| Survivin | + | − | − | − | + | − | + | − | + | − |
| MAGE-A8 | + | + | + | − | + | − | − | − | + | + |
| CAGE1 | + | + | + | + | − | + | − | − | − | + |
| SPAG9 | + | + | − | − | − | + | + | − | + | + |
| FBXO39 | + | + | − | − | + | + | + | + | + | + |

| | | | | | |
|---|---|---|---|---|---|
| OPA: | 86% | 86% | 14% | 57% | 71% |

| | |
|---|---|
| OPA Full: | 64% |

We compared the HLAT Scores and the number of antigens with the measured immune responses (FIG. 9). We found positive correlation between the HLAT Score and the number of antigens with immune responses. However, we do not expect significant correlation with such a small number of measurements (n=10) and because the HLAT Score considers the predicted epitope bindings for 48 antigens while the immune responses were measured for only 7 antigens out of the 48. This analysis therefore enables to show correlation but provides low statistical power.)

### Example 12—Comparison of the HLAT Score Based Classification and HLA-Score Based Classification

TABLE 17
HLAT Score based classification:

| | Co- | RR | | | | |
|---|---|---|---|---|---|---|
| Cancer | hort | Risk | Protected | | | |
| type | Size | Group* | Group* | RRₑₓₜᵣₑₘᵢₜᵢₑₛ | AUC | p |
| Melanoma | 513 | 1.73 | 0.31 | 5.53 | 0.65 | <0.001 |
| Lung (non- | 370 | 1.46 | 0.54 | 2.72 | 0.61 | <0.001 |
| small cell) | | | | | | |
| Renal cell | 129 | 1.59 | 0.58 | 2.74 | 0.60 | <0.001 |
| Colorectal | 121 | 1.83 | 0.61 | 2.97 | 0.62 | <0.001 |
| Bladder | 87 | 1.77 | 0.63 | 2.80 | 0.61 | <0.001 |
| Glioma | 82 | 1.36 | 0.79 | 1.71 | 0.57 | 0.017 |
| Head and | 58 | 1.02 | 0.17 | 5.90 | 0.54 | 0.15 |
| neck | | | | | | |

TABLE 18
HLA-score based classification:

| | Co- | RR | | | | |
|---|---|---|---|---|---|---|
| Cancer | hort | Risk | Protected | | | |
| type | Size | Group* | Group* | RRₑₓₜᵣₑₘᵢₜᵢₑₛ | AUC | p |
| Melanoma | 513 | 2.34 | 0.41 | 5.69 | 0.69 | <0.001 |
| Lung (non- | 370 | 1.84 | 0.41 | 4.49 | 0.66 | <0.001 |
| small cell) | | | | | | |
| Renal cell | 129 | 1.73 | 0.51 | 3.41 | 0.63 | <0.001 |
| Colorectal | 121 | 1.28 | 0.55 | 2.35 | 0.55 | 0.008 |
| Bladder | 87 | 1.89 | 0.46 | 4.14 | 0.66 | <0.001 |
| Glioma | 82 | 1.83 | 0.48 | 3.81 | 0.63 | <0.001 |
| Head and | 58 | 1.21 | 0.51 | 2.38 | 0.62 | 0.001 |
| neck | | | | | | |

As can be seen, HLAT Score based classification is better in case of colorectal cancer, while HLA-score based classification works better in case of head and neck cancer.

### Example 13—Genetic Differences in Ethnic Populations and its Association with Risk of Cancer

To further demonstrate that the HLA genotype influences the risk of developing cancer also on population level, we investigated its relationship with country-specific incidence rates. We hypothesized that the average HLA-score, i.e. the cancer-specific T-cell responses of a population with a high incidence rate of melanoma would be substantially lower than the HLA-score of a population with a low incidence rate. Therefore, we determined the HLA-scores for subjects representative for 59 different countries. We found that subjects in the Far East Asian and Pacific region had considerably higher HLA-scores (range 75-140) and lower incidence rates (range 0.4-3.4) than subjects of European or US origin (range 50 and 90) where the incidence rate is the highest (range 12.6-13.8) (FIG. 10). Focusing on the US population, the incidence rate of 1.5 per 100,000 persons for both Taiwan and Asia-Pacific islanders in the USA is significantly lower than for the general USA population (21.1 per 100,000 per year), confirming our results. Incidence rates were available by country while HLA genotype data were available by ethnicity. Therefore, we could obtain pairs of observations only for those countries that have a dominant ethnicity. We identified 20 countries with HLA genotype data from dominant ethnicities (highlighted with black on FIG. 10), for which we determined the mean HLA-scores and compared them with the incidence rates of melanoma. We found a significant correlation between the incidence rates of melanoma and average HLA-scores (FIG. 11). The correlation coefficient R²=0.5005 is highly significant (p<0.001) with the given number of points (n=20; degree of freedom, df=18). The countries with low and high melanoma incidence rates are well separated by an apparent HLA-score of >80 threshold, which is consistent with the threshold values separating low and high risk subjects in the US (HLA-score≥96, FIG. 11).

These results suggest that the HLA genotypes of subjects influence the incidence rate of melanoma in different ethnic populations and consistently suggest that the HLA-score could be used to determine the immunogenetic risk for melanoma.

### Example 14—HLA-Score of CLL Associated HLAs

A*02:01, C*05:01, C*07:01 are HLA alleles that are associated with CLL (chronic lymphocytic leukemia) (Gragert et al, 2014) meaning, that subjects having any of these HLA class I alleles have increased risk of developing CLL. During the HLA-score training, we observed that subjects in the training population having any of these HLAs have significantly less HLATs for the analysed 48 TSAs than subjects not having these HLAs. Table 19 shows the average HLAT numbers for the 48 TSAs in case of the 9 most frequent HLA alleles. However, these few HLA alleles can be found only in a small fraction of the population, and thus, the information that can be gained from the association between cancer and these few alleles cannot be used for subjects not having any of these alleles. On the other hand, the HLA score method assigns an informative score to all subjects and therefore can be used to classify the entire population. Therefore, the HLA score method provides better classification than a method using only information about association between individual HLA alleles and cancer.

TABLE 19
HLAT analysis of individuals having one of the CLL risk
increasing HLA A*02:01 or C*05:01 or C*07:01 alleles.

| | Average HLAT number | | |
|---|---|---|---|
| | Subjects having | Subjects | |
| | HLA*02:01 or | not having | t-test |
| HLA name | C*05:01 or C*07:01 | this HLA | p values |
| HLA-A*01:01 | 181.0 | 401.3 | 1.1503E−27 |
| HLA-A*02:01 | 325.6 | 403.6 | 8.67296E−09 |
| HLA-A*03:01 | 143.5 | 405.0 | 2.88788E−68 |
| HLA-A*33:03 | 101.5 | 385.7 | 0.720659487 |
| HLA-B*07:02 | 193.2 | 399.4 | 1.01724E−65 |
| HLA-B*08:01 | 115.5 | 393.1 | 6.31134E−36 |
| HLA-B*44:02 | 192.2 | 393.7 | 2.85151E−48 |
| HLA-C*05:01 | 150.2 | 391.8 | 8.36983E−54 |
| HLA-C*07:01 | 164.6 | 407.4 | 6.53173E−70 |

Example-15—One Allele or a Non-Complete HLA Genotype is not Appropriate to Determine Genetic Risk

It is known that Epstein-Barr virus (EBV) infection can induce undifferentiated nasopharyngeal carcinoma (UNPC). Pasini et al. analysed 82 Italian UNPC patients and 286 bone marrow donors from the same population and observed that some conserved alleles, A*0201, B*1801, and B*3501 HLA capable to bind to some EBV epitopes in the given region are underrepresented in UNPC subjects (Pasini E et al. Int. J. Cancer: 125, 1358-1364 (2009)). The investigation of the frequent alleles in the population, however is a completely different approach from the investigation of immune response inducing real target HLA-combinations, like HLAT pool analysis of the individuals. Since the latter suggests the potential of the person to produce diseased cell killing T cell repertoire, a mechanism explaining immunogenetic “advance” or risk. Furthermore, they found additive effect on protective HLA alleles. However, they did not infer if these HLA alleles can bind the same epitope or different epitopes on different EBV antigens. They also found HLA alleles which are positively associated to UNPC, however, they could not measure decreased ability of these HLA alleles to bind EBV epitopes. They considered only antigens from EBV, therefore their methods cannot be generalized to other cancers. Since even the most frequent HLA alleles cover only a limited fraction of the entire population, diagnostic devices cannot be constructed based on only them. For example, a device based on only the A*02:01 allele could have only an AUC value of 0.573 (FIG. 12). The combined haplotype A*02:01/B*18:01 is even rarer, and despite of the high OR value, a device based on the analysis of that single ‘haplotype’ would have only an AUC value of 0.556. That means, that it cannot significantly separate the population consisting of 82 UNPC patients from the background of 286 subjects, the transformed Z value is 1.65, the corresponding p-value (for one sided testing) is 0.06.

### Example 16—Study Design of OBERTO Phase I/II Clinical Trial and Preliminary Safety Data

OBERTO trial is a Phase I/II tria of PolyPEPI1018 Vaccine and CDx for the Treatment of Metastatic Colorectal Cancer (NCT03391232). Study design is shown on FIG. 13.

### Enrollment Criteria

Histologically confirmed metastatic adenocarcinoma originating from the colon or the rectum
Presence of at least 1 measurable reference lesion according to RECIST 1.1
PR or stable disease during first-line treatment with a systemic chemotherapy regimen and 1 biological therapy regimen
Maintenance therapy with a fluoropyrimidine (5-fluorouracil or capecitabine) plus the same biologic agent (bevacizumab, cetuximab or panitumumab) used during induction, scheduled to initiate prior to the first day of treatment with the study drug
Last CT scan at 3 weeks or less before the first day of treatment

### Subject Withdrawal and Discontinuation.

During the initial study period (12 W), if a patient experiences disease progression and needs to start a second-line therapy, the patient will be withdrawn from the study.
During the second part of the study (after 2nd dose) if a patient experiences disease progression and needs to start a second-line therapy, the patient will remain in the study, receive the third vaccination as scheduled and complete follow-up.
Transient local erythema and edema at the site of vaccination were observed as expected, as well as a flu-like syndrome with minor fever and fatigue. These reactions are already well-known for peptide vaccination and usually are associated with the mechanism of action, because fever and flu-like syndrome might be the consequence and sign for the induction of immune responses (this is known as typical vaccine reactions for childhood vaccinations).
Only one serious adverse event (SAE) “possibly related” to the vaccine was recorded (Table 20).
One dose limiting toxicity (DLT) not related to the vaccine occurred (syncope). Safety results are summarized in Table 19.

TABLE 20
Serious adverse events reported in the OBERTO clinical trial.
No related SAE occurred (only 1 “possibly related”).

| Patient ID | SAE | Relatedness |
|---|---|---|
| 010001 | Death due to disease progression | Unrelated |
| 010004 | Embolism | Unlikely Related |
| 010004 | Abdominal pain | Unrelated |
| 010007 | Bowel Obstruction | Unrelated |
| 020004 | Non-Infectious Acute Encephalitis | Possibly Related |

### Example 17—Expression Frequency Based Target Antigen Selection During Vaccine Design and It's Clinical Validation for mCRC

Shared tumor antigens enable precise targeting of all tumor types—including the ones with low mutational burden. Population expression data collected previously from 2,391 CRC biopsies represents the variability of antigen expression in CRC patients worldwide (FIG. 14A).

PolyPEPI1018 is a peptide vaccine we designed to contain 12 unique epitopes derived from 7 conserved testis specific antigens (TSAs) frequently expressed in mCRC. In our model we supposed, that by selecting the TSA frequently expressed in CRC, the target identification will be correct and will eliminate the need for tumor biopsy. We have calculated that the probability of 3 out of 7 TSAs being expressed in each tumor is greater than 95%. (FIG. 14B)

In a phase I study we evaluated the safety, tolerability and immunogenicity of PolyPEPI1018 as an add-on to maintenance therapy in subjects with metastatic colorectal cancer (mCRC) (NCT03391232) (See also in Example 4).

Immunogenicity measurements proved pre-existing immune responses and indirectly confirmed target antigen expression in the patients. Immunogenicty was measured with enriched Fluorospot assay (ELISPOT) from PBMC samples isolated prior to vaccination and in different time points following a following single immunization with PolyPEPI1018 to confirm vaccine-induced T cell responses; PBMC samples were in vitro stimulated with vaccine-specific peptides (9mers and 30mers) to determine vaccine-induced T cell responses above baseline. In average 4, at least 2 patients had pre-existing CD8 T cell responses against each target antigen (FIG. 14C). 7 out of 10 patients had pre-existing immune responses against at least 1 antigen (average 3) (FIG. 14D). These results provide proof for the proper target selection, because CD8+ T cell response for a CRC specific target TSA prior to vaccination with PolyPEPI1018 vaccine confirms the expression of that target antigen in the analyzed patient. Targeting the real (expressed) TSAs is the prerequisite for an effective tumor vaccine.

### Example 18—Pre-Clinical and Clinical Immunogenicity of PolyPEPI1018 Vaccine Proves Proper Peptide Selection

PolyPEPI1018 vaccine contains six 30mer peptides, each designed by joining two immunogenic 15mer fragments (each involving a 9mer PEPI, consequently there are 2 PEPIs in each 30mer by design) derived from 7 TSAs (FIG. 15). These antigens are frequently expressed in CRC tumors based on analysis of 2,391 biopsies (FIG. 14).

Preclinical immunogenicity results calculated for the Model Population (n=433) and for a CRC cohort (n=37) resulted in 98% and 100% predicted immunogenicity based on PEPI test predictions and this was clinically proved in the OBERTO trial (n=10), with immune responses measured for at least one antigen in 90% of patients. More interestingly, 90% of patients had vaccine peptide specific immune responses against at least 2 antigens and 80% had CD8+ T cell response against 3 or more different vaccine antigens, showing evidence for appropriate target antigen selection during the design of PolyPEPI1018. CD4+ T cell specific and CD8+ T cell specific clinical immunogenicity is detailed in Table 21. High immune response rates were found for both effector and memory effector T cells, both for CD4+ and CD8+ T cells, and 9 of 10 patients' immune responses were boosted or de novo induced by the vaccine. Also, the fractions of CRC-reactive, polyfunctional CD8+ and CD4+ T cells have been increased in patient's PBMC after vaccination by 2.5- and 13-fold, respectively.

TABLE 21
Clinical immunogenicity results for PolyPEPI1018 in mCRC.

| Immunological responses | % Patients (n) | |
|---|---|---|
| CD4+ T cell responses | 100% | (10/10) |
| CD8+ T cell responses against ≥3 antigens | 80% | (8/10) |
| Both CD8+ and CD4+ T cell responses | 90% | (9/10) |
| Ex vivo detected CD8+ T cell response | 71% | (5/7) |
| Ex vivo detected CD4+ T cell response | 86% | (6/7) |
| Average increase of the fraction of | 0.39% | |
| polyfunctional (IFN-γ and TNF-α positive) | | |
| CD8+ T cells compared to pre-vaccination | | |
| Average increase of the fraction of | 0.066% | |
| polyfunctional (IL-2 and TNF-α positive) | | |
| CD4+ T cells compared to pre-vaccination | | |

### Example 19—Clinical Response for PolyPEPI1018 Treatment

The OBERTO clinical trial (NCT03391232), that has been further described in Examples 4, 16, 17 and 18 was analyzed for preliminary objective tumor response rates (RECIST 1.1) (FIG. 16). Of the eleven vaccinated patients on maintenance therapy, 5 had stable disease (SD) at the time point of the preliminary analysis (12 weeks), 3 experienced unexpected tumor responses (partial response, PR) observed on treatment (maintenance therapy+vaccination) and 3 had progressed disease (PD) according to RECIST 1.1 criteria. Stable disease as best response was achieved in 69% of patients on maintenance therapy (capecitabine and bevacizumab). Patient 020004 had durable treatment effect after 12 weeks, and patient 010004 had long lasting treatment effect, qualified for curative surgery. Following the 3^{rd} vaccination this patient had no evidence of disease thus being complete responder, as shown on the swimmer plot on FIG. 16.

After one vaccination, ORR was 27%, DCR was 63%, and in patients receiving at least 2 doses (out of the 3 doses), 2 of 5 had ORR (40%) and DCR was as high as 80% (SD+PR+CR in 4 out of 5 patients) (Table 22).

TABLE 22
Clinical response for PolyPEPI1018 treatment
after ≥1 and ≥2 vaccination dose

| Number of | Objective Response Rate | Disease Control Rate |
|---|---|---|
| vaccination dose | (CR + PR) | (SD + PR + CR) |
| ≥1 | 27% (3/11) | 63% (7/11) |
| ≥2 | 40% (2/5) | 80% (4/5) |

Based on the data of the 5 patients receiving multiple doses of PolyPEPI1018 vaccine in the OBERTO-101 clinical trial, preliminary data suggests that higher AGP count (>2) is associated with longer PFS and elevated tumor size reduction (FIGS. 14B and C).

### Example 20—Personalised Immunotherapy (PIT) Design and Treatment for Ovarian-, Breast- and Colorectal Cancer

This Example provides proof of concept data from 4 metastatic cancer patients treated with personalized immunotherapy vaccine compositions to support the principals of binding of epitopes by multiple HLAs of a subject to induce cytotoxic T cell responses, on which the present disclosure is partly based on.

Composition for Treatment of Ovarian Cancer with P0001-PIT (Patient-A)

This example describes the treatment of an ovarian cancer patient with a personalised immunotherapy composition, wherein the composition was specifically designed for the patient based on her HLA genotype based on the disclosure described herein.

The HLA class I and class II genotype of a metastatic ovarian adenocarcinoma cancer patient (Patient-A) was determined from a saliva sample.

To make a personalized pharmaceutical composition for Patient-A thirteen peptides were selected, each of which met the following two criteria: (i) derived from an antigen that is expressed in ovarian cancers, as reported in peer reviewed scientific publications; and (ii) comprises a fragment that is a T cell epitope capable of binding to at least three HLA class I of Patient-A (Table 23). In addition, each peptide is optimized to bind the maximum number of HLA class II of the patient.

TABLE 23
Personalized vaccine of ovarian cancer Patient-A.

| POC01 | | | | MAX | MAX | Seq |
|---|---|---|---|---|---|---|
| vaccine for | Target | Antigen | | HLA | HLA | ID |
| Patient-A | Antigen | Expression | 20 mer peptides | classI | classII | NO |
| POC01_P1 | AKAP4 | 89% | NSLQKQLQAVLQWIAASQFN | 3 | 5 | 1 |
| POC01_P2 | BORIS | 82% | SGDERSDEIVLTVSNSNVEE | 4 | 2 | 2 |
| POC01_P3 | SPAG9 | 76% | VQKEDGRVQAFGWSLPQKYK | 3 | 3 | 3 |
| POC01_P4 | OY-TES-1 | 75% | EVESTPMIMENIQELIRSAQ | 3 | 4 | 4 |
| POC01_P5 | SP17 | 69% | AYFESLLEKREKTNFDPAEW | 3 | 1 | 5 |
| POC01_P6 | WT1 | 63% | PSQASSGQARMFPNAPYLPS | 4 | 1 | 6 |
| POC01_P7 | HIWI | 63% | RRSIAGEVASINEGMTRWES | 3 | 4 | 7 |
| POC01_P8 | PRAME | 60% | MQDIKMILKMVQLDSIEDLE | 3 | 4 | 8 |
| POC01_P9 | AKAP-3 | 58% | ANSVVSDMMVSIMKTLKIQV | 3 | 4 | 9 |
| POC01_P10 | MAGE-A4 | 37% | REALSNKVDELAHFLLRKYR | 3 | 2 | 10 |
| POC01_P11 | MAGE-A9 | 37% | ETSYEKVINYLVMLNAREPI | 3 | 4 | 11 |
| POC01_P12a | MAGE-A10 | 52% | DVKEVDPTGHSFVLVTSLGL | 3 | 4 | 12 |
| POC01_P12b | BAGE | 30% | SAQLLQARLMKEESPVVSWR | 3 | 2 | 13 |

Eleven PEPI3 peptides in this immunotherapy composition can induce T cell responses in Patient-A with 84% probability and the two PEPI4 peptides (P0001-P2 and P0001-P5) with 98% probability, according to the validation of the PEPI test shown in Table 4. T cell responses target 13 antigens expressed in ovarian cancers. Expression of these cancer antigens in Patient-A was not tested. Instead the probability of successful killing of cancer cells was determined based on the probability of antigen expression in the patient's cancer cells and the positive predictive value of the ≥1 PEPI3+ test (AGP count). AGP count predicts the effectiveness of a vaccine in a subject: Number of vaccine antigens expressed in the patient's tumor (ovarian adenocarcinoma) with PEPI. The AGP count indicates the number of tumor antigens that the vaccine recognizes and induces a T cell response against the patient's tumor (hit the target). The AGP count depends on the vaccine-antigen expression rate in the subject's tumor and the HLA genotype of the subject. The correct value is between 0 (no PEPI presented by any expressed antigen) and maximum number of antigens (all antigens are expressed and present a PEPI).

The probability that Patient-A will express one or more of the 13 antigens is shown in FIG. 17. AGP95 (AGP with 95% probability)=5, AGP50 (the mean—expected value—of the discrete probability distribution)=7.9, mAGP (probability that AGP is at least 2)=100%, AP=13.

A pharmaceutical composition for Patient-A may be comprised of at least 2 from the 13 peptides (Table 23), because the presence in a vaccine or immunotherapy composition of at least two polypeptide fragments (epitopes) that can bind to at least three HLAs of an individual (≥2 PEPI3+) was determined to be predictive for a clinical response. The peptides are synthetized, dissolved in a pharmaceutically acceptable solvent and mixed with an adjuvant prior to injection. It is desirable for the patient to receive personalized immunotherapy with at least two peptide vaccines, but preferable more to increase the probability of killing cancer cells and decrease the chance of relapse.

For treatment of Patient-A, the 13 peptides were formulated as 4×3 or 4 peptide (P0001/1, P0001/2, P0001/3, P0001/4). One treatment cycle is defined as administration of all 13 peptides within 30 days.

### Patient History:

Diagnosis: Metastatic ovarian adenocarcinoma

### Age: 51

Family anamnesis: colon and ovary cancer (mother) breast cancer (grandmother)

### Tumor Pathology:

2011: first diagnosis of ovarian adenocarcinoma; Wertheim operation and chemotherapy; lymph node removal
2015: metastasis in pericardial adipose tissue, excised
2016: hepatic metastases
2017: retroperitoneal and mesenteric lymph nodes have progressed; incipient peritoneal carcinosis with small accompanying ascites

### Prior Therapy:

### 2012: Paclitaxel-carboplatin (6×)

### 2014: Caelyx-carboplatin (1×)

2016-2017 (9 months): Lymparza (Olaparib) 2×400 mg/day, oral
2017: Hycamtin inf. 5×2.5 mg (3× one seria/month)

PIT vaccine treatment began on 21 Apr. 2017. FIG. 18.

2017-2018: Patient-A received 8 cycles of vaccination as add-on therapy, and lived 17 months (528 days) after start of the treatment. During this interval, after the 3^{rd} and 4^{th} vaccine treatment she experienced partial response as best response. She died in October 2018.

An interferon (IFN)-γ ELISPOT bioassay confirmed the predicted T cell responses of Patient-A to the 13 peptides. Positive T cell responses (defined as >5 fold above control, or >3 fold above control and >50 spots) were detected for all 13 20-mer peptides and all 13 9-mer peptides having the sequence of the PEPI of each peptide capable of binding to the maximum HLA class I alleles of Patient-A (FIG. 19).

Patient’ tumor MRI findings (Baseline Apr. 15, 2016) (BL: baseline for tumor response evaluation on FIG. 20)
Disease was confined primarily to liver and lymph nodes. The use of MRI limits detection of lung (pulmonary) metastasis
May 2016-January 2017: Olaparib treatment (FU1: follow up 1 on FIG. 20)
Dec. 25, 2016 (before PIT vaccine treatment) There was dramatic reduction in tumor burden with confirmation of response obtained at (FU2: follow up 2 on FIG. 20)
January-March 2017—TOPO protocol (topoisomerase)
Apr. 6, 2017 (FU3 on FIG. 20) demonstrated regrowth of existing lesions and appearance of new lesions leading to disease progression. Peritoneal carcinomatosis with increased amount of ascites. Progressive hepatic tumor and lymph node

### Apr. 21, 2017 START PIT

Jul. 26, 2017 (after the 2^{nd} Cycle of PIT): (FU4 on FIG. 20) Progression/Pseudo-Progression
Rapid progression in lymph nodes, hepatic, retroperitoneal and thoracic areas, significant pleural fluid and ascites. Initiate Carboplatin, Gemcitabine, Avastin.
   Sep. 20, 2017 (after 3 Cycles of PIT): (FU5 on FIG. 20) Partial Response
Complete remission in the pleural region/fluid and ascites
Remission in hepatic, retroperitoneal area and lymph nodes
The findings suggest pseudo progression.
   Nov. 28, 2017 (after 4 Cycles of PIT): (FU6 on FIG. 20) Partial Response
Complete remission in the thoracic region. Remission in hepatic, retroperitoneal area and lymph nodes

### Apr. 13, 2018: Progression

Complete remission in the thoracic and retroperitoneal regions. Progression in hepatic centers and lymph nodes
   Jun. 12, 2018: Stable disease
Complete remission in the thoracic and retroperitoneal regions. Minimal regression in hepatic centers and lymph nodes

### July 2018: Progression

October 2018: Patient-A died
Partial MRI data for Patient-A is shown in Table 24 and FIG. 20.

TABLE 24
Summary Table of Lesions Responses

| Lesion/ | Baseline | FU1 | FU2 | FU3 | FU4 | FU5 | FU6 | Best | PD |
|---|---|---|---|---|---|---|---|---|---|
| Time | (% Δ | (% Δ | (% Δ | (% Δ | (% Δ | (% Δ | (% Δ | Response | Time |
| Point | from BL) | from BL) | from BL) | from BL) | from BL) | from BL) | from BL) | Cycle | Point |
| TL1 | NA | −56.1 | −44.4 | −44.8 | +109.3 | −47.8 | −67.3 | FU6 | FU4 |
| TL2 | NA | −100.0 | −100.0 | −47.1 | −13.1 | −100.0 | −100.0 | FU1 | FU3 |
| TL3 | NA | −59.4 | −62.3 | −62.0 | −30.9 | −66.7 | −75.9 | FU6 | FU4 |
| TL4 | NA | −65.8 | −100.0 | −100.0 | −100.0 | −100.0 | −100.0 | FU2 | NA |
| SUM | NA | −66.3 | −76.0 | −68.9 | −23.5 | −78.2 | −85.2 | FU6 | FU4 |

### Design, Safety and Immunogenicity of Personalised Immunotherapy Composition PBRC01 for Treatment of Metastatic Breast Cancer (Patient-B)

The HLA class I and class II genotype of metastatic breast cancer Patient-B was determined from a saliva sample. To make a personalized pharmaceutical composition for Patient-B twelve peptides were selected, each of which met the following two criteria: (i) derived from an antigen that is expressed in breast cancers, as reported in peer reviewed scientific publications; and (ii) comprises a fragment that is a T cell epitope capable of binding to at least three HLA class I of Patient-B (Table 25). In addition, each peptide is optimized to bind the maximum number of HLA class II of the patient. The twelve peptides target twelve breast cancer antigens. The probability that Patient-B will express one or more of the 12 antigens is shown in FIG. 21.

TABLE 25
12 peptides for Patient-B breast cancer patient

| BRC01 | | | | MAXHL | MAXHL | Seq |
|---|---|---|---|---|---|---|
| vaccine | Target | Antigen | | A | A Class | ID |
| peptides | Antigen | Expression | 20 mer peptide | Class I | II | NO |
| PBRC01_cP1 | FSIP1 | 49% | ISDTKDYFMSKTLGIGRLKR | 3 | 6 | 14 |
| PBRC01_cP2 | SPAG9 | 88% | FDRNTESLFEELSSAGSGLI | 3 | 2 | 15 |
| PBRC01_cP3 | AKAP4 | 85% | SQKMDMSNIVLMLIQKLLNE | 3 | 6 | 16 |
| PBRC01_cP4 | BORIS | 71% | SAVFHERYALIQHQKTHKNE | 3 | 6 | 17 |
| PBRC01_cP5 | MAGE-A11 | 59% | DVKEVDPTSHSYVLVTSLNL | 3 | 4 | 18 |
| PBRC01_cP6 | NY-SAR-35 | 49% | ENAHGQSLEEDSALEALLNF | 3 | 2 | 19 |
| PBRCO1_cP7 | HOM-TES-85 | 47% | MASFRKLTLSEKVPPNHPSR | 3 | 5 | 20 |
| PBRC01_cP8 | NY-BR-1 | 47% | KRASQYSGQLKVLIAENTML | 3 | 6 | 21 |
| PBRC01_cP9 | MAGE-A9 | 44% | VDPAQLEFMFQEALKLKVAE | 3 | 8 | 22 |
| PBRC01_cP10 | SCP-1 | 38% | EYEREETRQVYMDLNNNIEK | 3 | 3 | 23 |
| PBRC01_cP11 | MAGE-Al | 37% | PEIFGKASESLQLVFGIDVK | 3 | 3 | 24 |
| PBRC01_cP12 | MAGE-C2 | 21% | DSESSFTYTLDEKVAELVEF | 4 | 2 | 25 |

Predicted efficacy: AGP95=4; 95% likelihood that the PIT Vaccine induces CTL responses against 4 TSAs expressed in the breast cancer cells of Patient-B. Additional efficacy parameters: AGP50=6.45, mAGP=100%, AP=12.

For treatment of Patient-B the 12 peptides were formulated as 4×3 peptide (PBR01/1, PBR01/2, PBR01/3, PBR01/4). One treatment cycle is defined as administration of all 12 different peptide vaccines within 30 days (FIG. 21C).

### Patient History:

2013: Diagnosis: breast carcinoma diagnosis; CT scan and bone scan ruled out metastatic disease.
2014: bilateral mastectomy, postoperative chemotherapy
2016: extensive metastatic disease with nodal involvement both above and below the diaphragm. Multiple liver and pulmonary metastases.

### Therapy:

### 2013-2014: Adriamycin-Cyclophosphamide and Paclitaxel

2017: Letrozole, Palbocichb and Gosorelin and PIT vaccine
2018: Worsening conditions, patient died in January

PIT vaccine treatment began on 7 Apr. 2017. treatment schedule of Patient-B and main characteristics of disease are shown in Table 26.

TABLE 26
Treatment and response of Patient-B

| | Date (2017) | | | | | |
|---|---|---|---|---|---|---|
| | Mar | May | Jun | Sep | Nov | |
| | PIT Vaccine | | | | | Dec |
| | Palbocyclib | | | | | |
| Treatment | Letrozole | | Anticancer drug | | | |
| regimen | Gosorelin | | treatment interruption | | | |
| Neutrophils | ND | 1.1 | 4.5 | 3.4 | 2.4 | 3 |
| (1.7-3.5/mm³) | | | | | | |
| CEA | 99 | 65 | 23 | 32 | 128 | 430 |
| (<5.0 ng/ml) | | | | | | |
| CA 15-3 | 322 | 333 | 138 | 76 | 272 | 230 |
| (<31.3 U/ml | | | | | | |
| T1: Right axillar | 15 mm & | 9 mm & | nd* | nd | nd | 6 mm & |
| lymph node | 11.6 SUVₘₐₓ | 2.0 SUVₘₐₓ | | | | 0 SUVₘₐₓ |
| T2: Right lung | 10 mm & | 7 mm & | nd | nd | nd | 4 mm & |
| metastasis | 4.8 SUVₘₐₓ | 0 SUVₘₐₓ | | | | 0 SUVₘₐₓ |
| Left iliac bone | Non | Regression | nd | nd | nd | Regression |
| metastasis | measurable | & 0 SUVₘₐₓ | | | | & 0 SUVₘₐₓ |
| | & 4.0 SUVₘₐₓ | | | | | |
| Multiple liver | Non | Partial | nd | nd | nd | Progression |
| metastases | measurable | regression | | | | & 16.8 SUVₘₐₓ |
| | & 11.5 SUVₘₐₓ | & 6.1 SUVₘₐₓ | | | | |

*no data

It was predicted with 95% confidence that 8-12 vaccine peptides would induce T cell responses in Patient-B. Peptide-specific T cell responses were measured in all available PBMC samples using an interferon (IFN)-γ ELISPOT bioassay (FIG. 22). The results confirmed the prediction: Nine peptides reacted positive demonstrating that T cells can recognize Patient-B's tumor cells expressing FISP1, BORIS, MAGE-A11, HOM-TES-85, NY-BR-1, MAGE-A9, SCP1, MAGE-A1 and MAGE-C2 antigens. Some tumor specific T cells were present after the 1^{st} vaccination and boosted with additional treatments (e.g. MAGE-A1) others induced after boosting (e.g. MAGE-A9). Such broad tumor specific T cell responses are remarkable in a late stage cancer patient.

### Patient-B History and Results

Mar. 7, 2017: Prior PIT Vaccine treatment
Hepatic multi-metastatic disease with truly extrinsic compression of the origin of the choledochal duct and massive dilatation of the entire intrahepatic biliary tract. Celiac, hepatic hilar and retroperitoneal adenopathy
March 2017: Treatment initiation—Letrozole, Palbociclib, Gosorelin & PIT Vaccine
May 2017: Drug interruption
May 26, 2017: After 1 cycle of PIT
83% reduction of tumor metabolic activity (PET CT) liver, lung lymphnodes and other metastases.
June 2017: Normalized Neutrophils values indicate Palbociclib interruption as affirmed by the patient

### 4 Months Delayed Rebound of Tumor Markers

March to May 2017: CEA and CA remained elevated consistently with the outcome of her anti-cancer treatment (Ban, Future Oncol 2018)
June to September 2017: CEA and CA decreased consistently with the delayed responses to immunotherapies

### Quality of Life

February to March 2017: Poor, hospitalized with jaundice

### April to October 2017: Excellent

November 2017: Worsening conditions (tumor escape?)
January 2018: Patient-B died.
Immunogenicity results are summarized in FIG. 22.

Clinical outcome measurements of the patient: One month prior to the initiation of PIT vaccine treatment PET CT documented extensive DFG avid disease with nodal involvement both above and below the diaphragm (Table 26). She had progressive multiple hepatic, multifocal osseous and pulmonary metastases and retroperitoneal adenopathy. Her intrahepatic enzymes were elevated consistent with the damage caused by her liver metastases with elevated bilirubin and jaundice. She accepted Letrozole, Palbociclib and Gosorelin as anti-cancer treatment. Two month after initiation of PIT vaccinations the patient felt very well and her quality of life normalized. In fact, her PET CT showed a significant morphometabolic regression in the liver, lung, bone and lymph node metastases. No metabolic adenopathy was identifiable at the supra-diaphragmatic stage.

The combination of Palblocyclib and the personalised vaccine was likely to have been responsible for the remarkable early response observed following administration of the vaccine. Palbocyclib has been shown to improve the activity of immunotherapies by increasing TSA presentation by HLAs and decreasing the proliferation of Tregs (Goel et al. Nature. 2017:471-475). The results of Patient-B treatment suggest that PIT vaccine may be used as add-on to the state-of-art therapy to obtain maximal efficacy.

Patient-B's tumor biomarkers were followed to disentangle the effects of state-of-art therapy from those of PIT vaccine. Tumor markers were unchanged during the initial 2-3 months of treatment then sharply dropped suggesting of a delayed effect, typical of immunotherapies (Table 26). Moreover, at the time the tumor biomarkers dropped the patient had already voluntarily interrupted treatment and confirmed by the increase in neutrophil counts.

After the 5^{th} PIT treatment the patient experienced symptoms. The levels of tumor markers and liver enzymes were increased again. 33 days after the last PIT vaccination, her PET CT showed significant metabolic progression in the liver, peritoneal, skeletal and left adrenal site confirming the laboratory findings. The discrete relapse in the distant metastases could be due to potential immune resistance; perhaps caused by downregulation of both HLA expression that impairs the recognition of the tumor by PIT induced T cells. However, the PET CT had detected complete regression of the metabolic activity of all axillary and mediastinal axillary supra-diaphragmatic targets (Table 26). These localized tumor responses may be accounted to the known delayed and durable responses to immunotherapy, as it is unlikely that after anti-cancer drug treatment interruption these tumor sites would not relapse.

Personalised Immunotherapy Composition for Treatment of a Patient with Metastatic Breast Carcinoma (Patient-C)

PIT vaccine similar in design to that described for Patient-A and Patient-B was prepared for the treatment of a patient (Patient-C) with metastatic breast carcinoma. PIT vaccine contained 12 PEPIs. The PIT vaccine has a predicted efficacy of AGP=4. The patient's treatment schedule is shown in FIG. 23.

### Tumor Pathology

2011 Original tumor: HER2-, ER+, sentinel lymph node negative
2017 Multiple bone metastases: ER+, cytokeratin 7+, cytokeratin 20−, CA125−, TTF1−, CDX2−

### Treatments

2011 Wide local resection, sentinel lymph nodes negative; radiotherapy
2017—Anti-cancer therapy (Tx): Letrozole (2.5 mg/day), Denosumab;
Radiation (Rx): one bone
PIT vaccine (3 cycles) as add-on to standard of care

Bioassay confirmed positive T cell responses (defined as >5 fold above control, or >3 fold above control and >50 spots) to 11 out of the 12 20-mer peptides of the PIT vaccine and 11 out of 12 9-mer peptides having the sequence of the PEPI of each peptide capable of binding to the maximum HLA class I alleles of the patient (FIG. 24). Long-lasting memory T-cell responses were detected after 14 months of the last vaccination (FIG. 24C-D).

### Treatment Outcome

Clinical results of treatment of Patient-C are shown in Table 27. Patient-C has partial response and signs of healing bone metastases.

TABLE 27
Clinical results of treatment of breast cancer Patient-C

| | | +70 days* | +150 days* | +388 days* |
|---|---|---|---|---|
| | Before PIT | (10 w) | (21 w) | (55 w) |
| Bone | Met. breast | Not done | RIB5 | Not done |
| Biopsy | cancer DCIS | | is negative | |
| PET CT | Multiple | Only RIB5 is | Not done | Not done |
| | metastases | DFG avid | | |
| CT | Multiple | Not done | Not done | Healing bone |
| | metastases | | | mets (sclerotic |
| | | | | foci) |
| CA-15-3 | 87 | 50 | 32 | 24 |

*After 3rd cycle of PIT vaccination

Immune responses are shown on FIG. 24. Predicted Immunogenicity, PEPI=12 (CI95% [8,12]
Detected Immunogenicity: 11 (20-mers) & 11 (9-mers) antigen specific T cell responses following 3 PIT vaccinations (FIG. 24A, B). After 4.5, 11 or 14 months of the last vaccination, PIT vaccine-specific immune response could still be detected (FIG. 24 C, D).
Personalised Immunotherapy Composition for Treatment of Patient with Metastatic Colorectal Cancer (Patient-D)

Tumor pathology

| | |
|---|---|
| 2017 | mCRC (MSS) with liver metastases, surgery of |
| (February) | primer tumor (in sigmoid colon). pT3 pN2b (8/16) |
| | M1. KRAS G12D, TP53-C135Y, KDR-Q472H, MET- |
| | T1010I mutations. SATB2 expression. EGFR wt, |
| | PIK3CA-I391M (non-driver). |
| 2017 (June) | Partial liver resection: KRAS-G12D (35G > A) NRAS |
| | wt, |
| 2018 (May) | 2^{nd} resection: SATB2 expression, lung metastases |
| | 3 → 21 Treatments |
| 2017 | FOLFOX-4 (oxaliplatin, Ca-folinate, 5-FU) → allergic |
| | reaction during 2^{nd} treatment |
| | DeGramont (5-FU + Ca-folinate) |
| 2018 (June) → | FOLFIRI plus ramucirumab, biweekly; |
| | chemoembolization |
| 2018 | PIT vaccination (13 patient-specific peptides, |
| (October) | 4 doses) as add-on to standard of care. |

The patient's treatment schedule is shown in FIG. 25.

### Treatment Outcome

Patient in good overall condition, disease progression in lungs after 8 months confirmed by CT.

Both PIT induced and pre-existing T cell responses were measured by enriched Fluorospot from PBMC, using 9mer and 20mer peptides for stimulation (FIG. 26).

Summary of immune response rate and immunogenicity results prove the proper design for target antigen selection as well as for the induction of multi-peptide targeting immune responses, both CD4+ and CD8+ specific ones.

TABLE 28
Summary table of immunological analysis of Patient A-D

Measured immunogenicity for the
different vaccine peptides*

| Patient ID | CD4+ T cells | CD8+ T cells |
|---|---|---|
| Patient-A | 13/13 (100%) | 13/13 (100%) |
| Patient-B | 9/12 (75%) | 1/12 (8%) |
| Patient-C | 11/12 (92%) | 11/12 (92%) |
| Patient-D | 7/13 (54%) | 13/13 (100%) |
| IRR (ratio of immune | 4/4 | 4/4 |
| responder patients) | | |
| Ratio of immunogenic | 10/12-13 | 10/12-13 |
| peptides (median) | | |

*Following 1-3 cycles of vaccination

1.-9. (canceled)

10. A method of determining a risk of an individual developing a cancer, the method comprising:
(i) identifying a human leukocyte antigen (HLA) class I genotype comprising six HLA alleles existing in the individual by obtaining a biological sample comprising DNA from the individual and performing a DNA sequencing assay on the biological sample;
(ii) obtaining an epitope prediction data set obtained by screening a first tumor associated antigen (TAA) protein with overlapping 9-mer peptides and identifying T-cell epitopes that bind to the individual's HLA Class I alleles;
(iii) repeating step (ii) for all possible 9-mer epitopes of the first TAA and, optionally
(iv) repeating step (iii) for up to 48 TAAs as provided in Tables 2 and 11;
(v) computing an HLA score for one of the six HLA alleles for the first TAA and, optionally for up to the 48 TAAs in Tables 2 and 11, wherein the score is based on frequency of the one of the six alleles participating in HLA-j tuples binding to the T-cell epitope and wherein there are
$$( \begin{matrix}k \\ j\end{matrix} ) = \frac{k !}{( k - j ) ! j !}$$
possible HLA allele j-mer combinations for the T-cell epitope where j is 1, 2, 3, 4, 5, or 6 and k is a number of autologous HLA alleles that can bind to the T-cell epitope;
(vi) calculating an HLA score for each of the six alleles of the individual for the first TAA and, optionally calculating an HLA score for up to 48 TAAs in Tables 2 and 11, and comparing the sum of all calculated HLA scores for the up to 48 TAAs of the individual to an average HLA score in a background population; and
(vii) identifying the individual as at higher risk of developing the cancer if the sum of the HLA score for the TAAs of the individual is higher than the average HLA score for the background population.

11. The method of claim 10, wherein j=3.

12. The method of claim 10, wherein a population not at higher risk of developing the cancer has similar demographics as the individual.

13. The method of claim 10, wherein the cancer is selected from melanoma, lung cancer, renal cell cancer, colorectal cancer, bladder cancer, glioma, head and neck cancer, ovarian cancer, non-melanoma skin cancer, prostate cancer, kidney cancer, stomach cancer, liver cancer, cervix uteri cancer, oesophagus cancer, non-Hodgkin lymphoma, leukemia, pancreatic cancer, corpus uteri cancer, lip cancer, oral cavity cancer, thyroid cancer, brain cancer, nervous system cancer, gallbladder cancer, larynx cancer, pharynx cancer, myeloma, nasopharynx cancer, Hodgkin lymphoma, testis cancer, breast cancer, gastric cancer, bladder cancer, colorectal cancer, renal cell cancer, hepatocellular cancer, pediatric cancer and Kaposi sarcoma.

14. The method of claim 10, further comprising treating the individual for the cancer if they are determined to be at a higher risk of developing the cancer.

15. The method of claim 14, wherein treating comprises administering, to the individual, a peptide comprising at least one fragment of the first TAA, which fragment comprises the first T-cell epitope.

16. The method of claim 15, wherein the at least one fragment of the first TAA is flanked at the N and/or C terminus by additional amino acids that are not part of the sequence of the first TAA.

17. The method of claim 16, wherein the at least one fragment of the first TAA is about 15 to 50 amino acids in length.

18. The method of claim 10, wherein the first TAA is selected from those listed in Table 2 or Table 11.

19. The method of claim 15, wherein the treating further comprises administering to the individual a cytotoxic chemotherapy, a non-cytotoxic chemotherapy, a radiation therapy, a targeted therapy, a checkpoint inhibitor therapy, or combinations thereof.

20. A method of determining a risk of an individual developing a cancer, the method comprising:
(i) identifying a human leukocyte antigen (HLA) class I genotype comprising six HLA alleles existing in the individual by obtaining a biological sample comprising DNA from the individual and performing a DNA sequencing assay on the biological sample;
(ii) obtaining an epitope prediction data set obtained by screening a first tumor associated antigen (TAA) protein with overlapping 9-mer peptides and identifying T-cell epitopes that bind to the individual's HLA Class I alleles;
(iii) repeating step (ii) for all possible 9-mer epitopes of the first TAA and, optionally (iv) repeating step (iii) for up to 48 TAAs as provided in Tables 2 and 11;
(v) computing a total number of HLA triplets (HLATs) in the individual that bind to the 9-mer epitopes of the first TAA and, optionally, for up to the 48 TAAs of Tables 2 and 11, from a total number of possible HLATs for the first TAA and, optionally, for up to the 48 TAAs of Tables 2 and 11;
(vi) calculating, for the individual, an HLAT Score for the first TAA and, optionally, for up to the 48 TAAs of Tables 2 and 11, and comparing the calculated HLAT Score to an average HLAT Score for the background population; and
(vii) identifying the individual as at higher risk of developing cancer if an HLAT score for the first TAA or any of the up to 48 TAAs in Tables 2 and 11 is lower than an HLAT score for the first TAA or any of the up to 48 TAAs in Tables 2 and 11 in the background population not at higher risk of developing the cancer.

21. The method of claim 20, wherein each TAA of the individual with an HLAT score that is not significantly different from the background population is weighted as zero (0).

22. The method of claim 20, wherein a population not at higher risk of developing the cancer has similar demographics as the individual.

23. The method of claim 20, wherein the cancer is selected from melanoma, lung cancer, renal cell cancer, colorectal cancer, bladder cancer, glioma, head and neck cancer, ovarian cancer, non-melanoma skin cancer, prostate cancer, kidney cancer, stomach cancer, liver cancer, cervix uteri cancer, oesophagus cancer, non-Hodgkin lymphoma, leukemia, pancreatic cancer, corpus uteri cancer, lip cancer, oral cavity cancer, thyroid cancer, brain cancer, nervous system cancer, gallbladder cancer, larynx cancer, pharynx cancer, myeloma, nasopharynx cancer, Hodgkin lymphoma, testis cancer, breast cancer, gastric cancer, bladder cancer, colorectal cancer, renal cell cancer, hepatocellular cancer, pediatric cancer and Kaposi sarcoma.

24. The method of claim 20, furthering comprising treating the individual for the cancer if they are determined to be at a higher risk of developing the cancer.

25. The method of claim 24, wherein the treating comprises administering to the individual a peptide comprising at least one fragment of the first TAA, which fragment comprises the first T-cell epitope.

26. The method of claim 25, wherein the at least one fragment of the first TAA is flanked at the N and/or C terminus by additional amino acids that are not part of the sequence of the first TAA.

27. The method of claim 26, wherein the at least one fragment of the first TAA is about 15 to 50 amino acids in length.

28. The method of claim 20, wherein the first TAA is selected from those listed in Table 2 or Table 11.

29. The method of claim 25, wherein the treating further comprises administering to the individual a cytotoxic chemotherapy, a non-cytotoxic chemotherapy, a radiation therapy, a targeted therapy, a checkpoint inhibitor therapy, or combinations thereof.


SEQUENCE LISTING (26 sequences)

SEQ ID NO: 1 - protein, 20 aa, artificial sequence
  POC01_P1 AKAP4
NSLQKQLQAVLQWIAASQFN

SEQ ID NO: 2 - protein, 20 aa, artificial sequence
  POC01_P2 BORIS
SGDERSDEIVLTVSNSNVEE

SEQ ID NO: 3 - protein, 20 aa, artificial sequence
  POC01_P3 SPAG9
VQKEDGRVQAFGWSLPQKYK

SEQ ID NO: 4 - protein, 20 aa, artificial sequence
  POC01_P4 OY-TES-1
EVESTPMIMENIQELIRSAQ

SEQ ID NO: 5 - protein, 20 aa, artificial sequence
  POC01_P5 SP17
AYFESLLEKREKTNFDPAEW

SEQ ID NO: 6 - protein, 20 aa, artificial sequence
  POC01_P6 WT1
PSQASSGQARMFPNAPYLPS

SEQ ID NO: 7 - protein, 20 aa, artificial sequence
  POC01_P7 HIWI
RRSIAGFVASINEGMTRWFS

SEQ ID NO: 8 - protein, 20 aa, artificial sequence
  POC01_P8 PRAME
MQDIKMILKMVQLDSIEDLE

SEQ ID NO: 9 - protein, 20 aa, artificial sequence
  POC01_P9 AKAP-3
ANSVVSDMMVSIMKTLKIQV

SEQ ID NO: 10 - protein, 20 aa, artificial sequence
  POC01_P10 MAGE-A4
REALSNKVDELAHFLLRKYR

SEQ ID NO: 11 - protein, 20 aa, artificial sequence
  POC01_P11 MAGE-A9
ETSYEKVINYLVMLNAREPI

SEQ ID NO: 12 - protein, 20 aa, artificial sequence
  POC01_P12a MAGE-A10
DVKEVDPTGHSFVLVTSLGL

SEQ ID NO: 13 - protein, 20 aa, artificial sequence
  POC01_P12b BAGE
SAQLLQARLMKEESPVVSWR

SEQ ID NO: 14 - protein, 20 aa, artificial sequence
  PBRC01_cP1 FSIP1
ISDTKDYFMSKTLGIGRLKR

SEQ ID NO: 15 - protein, 20 aa, artificial sequence
  PBRC01_cP2 SPAG9
FDRNTESLFEELSSAGSGLI

SEQ ID NO: 16 - protein, 20 aa, artificial sequence
  PBRC01_cP3 AKAP4
SQKMDMSNIVLMLIQKLLNE

SEQ ID NO: 17 - protein, 20 aa, artificial sequence
  PBRC01_cP4 BORIS
SAVFHERYALIQHQKTHKNE

SEQ ID NO: 18 - protein, 20 aa, artificial sequence
  PBRC01_cP5 MAGE-A11
DVKEVDPTSHSYVLVTSLNL

SEQ ID NO: 19 - protein, 20 aa, artificial sequence
  PBRC01_cP6 NY-SAR-35
ENAHGQSLEEDSALEALLNF

SEQ ID NO: 20 - protein, 20 aa, artificial sequence
  PBRC01_cP7 HOM-TES-85
MASFRKLTLSEKVPPNHPSR

SEQ ID NO: 21 - protein, 20 aa, artificial sequence
  PBRC01_cP8 NY-BR-1
KRASQYSGQLKVLIAENTML

SEQ ID NO: 22 - protein, 20 aa, artificial sequence
  PBRC01_cP9 MAGE-A9
VDPAQLEFMFQEALKLKVAE

SEQ ID NO: 23 - protein, 20 aa, artificial sequence
  PBRC01_cP10 SCP-1
EYEREETRQVYMDLNNNIEK

SEQ ID NO: 24 - protein, 20 aa, artificial sequence
  PBRC01_cP11 MAGE-A1
PEIFGKASESLQLVFGIDVK

SEQ ID NO: 25 - protein, 20 aa, artificial sequence
  PBRC01_cP12 MAGE-C2
DSESSFTYTLDEKVAELVEF

SEQ ID NO: 26 - protein, 30 aa, artificial sequence
  crc_P3 peptide 30aa
YVDEKAPEFSMQGLKDEKVAELVRFLLRKY